SEQUENCE LISTING (60 sequences)

SEQ ID NO: 1 - DNA, 6121 bp, Artificial Sequence
  Nucleotides 961381 to 967501 of GenBank Accession No. NG_001019 (Jan 10, 2006 version).
gtaagatgtttaagaaattaaacagtcttagggagagtttatgactgtattcaaaaagttttttaaattagcttgttatcccttcatgtgataactaatctcaaatactttttcgatacctcagagcattattttcataatgactgtgttcacaatctttttaggttaactcgttttctctttgtgattaaggagaaacactttgatattctgatagagtggccttcattttagtatttttcaagaccacttttcaactactcactttaggataagttttaggtaaaatgtgcatcattatcctgaattatttcagttaagcatgttagttggtggcataagagaaaactcaatcagatagtgctgaagacaggactgtggagacaccttagaaggacagattctgttccgaatcaccgatgcggcgtcagcaggactggcctagcggaggctctgggagggtggctgccaggcccggcctgggctttgggtctccccggactacccagagctgggatgcgtggcttctgctgccgggccgactggctgctcaggccccagcccttgttaatggacttggaggaatgattccatgccaaagctttgcaaggctcgcagtgaccaggcgcccgacatggtaagagacaggcagccgccgctgctgcatttgcttctcttaaaactttgtatttgacgtcttatttccactagaaggggaactggtcttaattgcttgatgaagagcaggagactcatttatgtgagtcttttgagtgaccattgtctgggtcactcccatttaactttccctaaagcccatttgaaggagaggtcgcacgagctgctccacaacctctgaatggggatggcatgggtaatgatgcttgagaacataccaagccccactggcatcgcccttgtctaagtcattgactgtaggtcatcatcgcacccttgaaagtagcccatgccttccaaagcgatttatggtaaatggcagaattttaagtggcaaattcagataaaatgcatttcttggttgtttccaatgatgactgttatctagagggaatttaaaggcaggggtttactgcagactcagaagggaggggatgctccgggaaggtggaggctctgagcatctcaataccctcctcttggtgcagaagatatgctgccacttctagagcaaggggacctgctcatttttatcacagcacaggctcctaaattcttggtctcattctcaagatgttttaatgactttaaagcagcaaagaaatattccacccaggtagtggagggtggtaatgattggtaatgctttggaaccaaaacccaggtggcgctggggcaggactgcagggaactggggtatcaagtagagggagacaaaagatggaagccagcctggctgtgcaggaacctggcaatgagatggctttagctgagacaagcaggtctggtgggctgaccatttctggccatgacaactccatccagctttcagaaatggactcagatgggcaaaactgacctaagctgacctagactaaacaaggctgaactgggctgagctgagctgaactgggctgagttgaactgggttgagctgagctgagctgagctgggctaagttgcaccaggtgagctgagctgagctgggcttggctgcactaagctgggctgagctgggcagggctgggctgagctgagctgggctgggctgagctgggctgggctgggctgggctgagcgggtctgagcggggctgagctgagctaggctgggctgggctgagctgggctgagctgggctgagctgggctgagcaaggctaggctgagctgggctgagctgagctgggccgagcaaggctaggctgagctgagctgagctgggctgcgctgagctgggctgggctgcgctgagctgggctgggctgagctgggctaggctgggctgagctgggctgagctaggctgggctgggctgggctgagcggggctgagcggggctgagctgagctaggctgggctgagcggggctgagctgagctaggctgggctgggctgggctgagccaagctgaaccgggttgagcgtgctgtgctgggctgagccaagctaggctgagctgagccaagttgagcttagctgggctgagctaacctgggcaaggctgagctgggctgagctaacctggactggggctgagctaacctgggcagagctgagctgggctgagctaacctggactggggctgagctaacctgggcagagctgagctgggctgagctaacctgggctggggctgagctaacctgggctgggctcagctgagctgagctacgctgggctgggctgggctgagccgagctgaactgggctgagcaggctgtgtcgggctgagccaagctgggccgagctcagcagagctgagccgagctgagcttagctgggctgagctaaccagggctgggctgagctgggctgagctgagctgagctgaactgggctgaacgggctgaggcgagcagggccgagctgagcagagctaagccgaggctgggctgggctaacctgggctgggccgagctgagcagagctaagccgaggctgggctgggctaacctgggctgggctgagctgagctgggttgggcagggctgggctgggctgagctaagctgaactagggtgagctgggccgagccaggctggtttgggctgagttgagctgacctggactggggctgagctaacctgggctgggctcagctgagctgagctacgctgggctgggctgggctgagccgagctgaactgggctgagcaggttgtatcaggctaagccaagctgggccaagctcagcagagccaagccgagctgagcttagctgggctgagctaaccagggctgggctgagctgggctgagctgagctgagctgaactgggctgaacgggctgaggcgagcagggccgagctgagcagagctaagccgaggctgggctgggctaacctgggctgggctgagctgagctgggttgggcaaggctgggctgggttgagttgggctgagctaagctgagctagggtgagctgggctgagccaggctggattgggctgagttgagctgacctcaactgagctgacctaggctgagctgagctgagctgaagtaggctgtgctgggctgagctgggatgacctgggctgggctgagctgatttgggctgggttgagcagacctgggctgagccgggttgagctgagctgaaccaggatgagctgggctgagctgagctgggctgggtggtccaggctgggctgacctggaccaggctgggccaggttgagctgggctaagccgagctgagctgagctgagctgggatgatctgggctgggctgggctgggctgagctgacctgggctgggctgggctgagctgagctgagctgagttaagctgagctgagctgaactgggctgtgctgagctaggctgggctgggctgagctgggctgggctgggctgagccagattgtgcctggctgaactgagctgggctaagctgagctgggctgagctgggctgagctgagctgggctgagtggggcggggctgagctgagccggactgggctgggctgggctcagctgagctgagctgaactgggctgggctgaactgggctgggctgagctgagctgaactgggctgggctgaactgggctgggctgagctgagcttggatgagctgggctgaactgggctgggttgagctgggctgggctgagttgagccaggctgatctgggctgagccgagctgggttaagccgagctgggttgggctgggctgggttgggctgggctgagccggactgggttgggctgagctgagctggactgggctgagctgagctggtctgggctggctgagctgagctgtgttgagccaagctaggctgggctgggctgagctgggctgaactgggctgagcggagctgagctgagctgggctgggttgagcagagctgggggctgagctgggctgggctgggctgagttaagctgggctgacctgggctgagttaagctgggctgacctgggctgagctaagctgggttgagccgatccgggctgggctgggctgggctgagccaggctaggttgagctgggctgggctgggctctgctgtgctgtgctgaacagggctgagctgaactgagctgagctgggctgagctgggctctgctgtgctgtgctgagcagggctgagctgaactgagctgagctgggttgagctaagctaggctgggctgggctgagctgggatgagctgggctgggctgggctgggctctgctgtgctgtgctgaacagggctgagctgaactgagctgagctgggctgagctgggctctgctgtgctgtgctgagcagggctgagctgaactgggctgagctgggctgagctgggctgagttgagcagagctgggttgagcagagctgggctgggctgggctgagttgagccaggctgacctgggctgagccaagctgggttgagccaaactgggctgggctgggctgagccaggctggattgagctgggctgggctgggctaagctgagccgagcaaggttgagcgagctgggctgggttgggctgagctgagctgggctgggctgagctgggctgtgctgcactgagctgggctgagctgggttgaactgtgctgagctgagctgggctgtgctgaactatgcttagctgggctgggctatactgggcttagctgggctgggctaaactgggcttagctgggctgggctatactgggcttagctgggctgggctaaactgggcttagctgggctgggctatactgggcttagctgggctgggctgagctgagatggtcttaggtggtctgagctcagctaggctgggctgagctggtctgagctcatctgagttgggctgagctgagcttggctttgctgagctggggtggggtgggctgggctggattgagctggcctgggctgggatgaactggattgagctggcctgggctgggatgaactggaggacatggcactgggccaatcttcatgatcttgttggacatagatggatagcctcagctgagtctacactgcgttccccatcacacccaccctccctatactcactcccaggcctgggttgtctgcctggggagacttcagggtagctggagtgtgactgagctgggggcagcagaagctgggctggagggactctattggctgcctgcggggtgtgtggctccaggcttcacattcaggtatgcaacctgggccctccagctgcatgtgctgggagctgagtgtgtgcagcacctacgtgctgatgcctcgggggaaagcaggcctggtccacccaaacctgagccctcagccattctgagcagggagccaggggcagtcaggcctcagagtgcagcagggcagccagctgaatggtggcagggatggctcagcctgctccaggagaccccaggtctgtccaggtgttcagtgctgggccctgcagcaggatgggccgaggcctgcagccccagcagccttggacaaagacctgaggcctcaccacggccccgccacccctgatagccatgacagtctgggctttggaggcctgcaggtgggctcggccttggtggggcagccacagcgggacgcaagtagtgagggcactcagaacgccactcagccccgacaggcagggcacgaggaggcagctcctcaccctccctttctcttttgtcctgcgggtcctcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacgg

SEQ ID NO: 2 - DNA, 81061 bp, Artificial Sequence
  Nucleotides 967201 to 1048261 of GenBank Accession No. NG_001019 (Jan 10, 2006 version).
tgtcctgcgggtcctcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttccaggtgagggccgggcccagccaccgggacagagagggagccgaagggggcgggagtggcgggcaccgggctgacacgtgtccctcactgcagtgattgccgagctgcctcccaaagtgagcgtcttcgtcccaccccgcgacggcttcttcggcaacccccgcaagtccaagctcatctgccaggccacgggtttcagtccccggcagattcaggtgtcctggctgcgcgaggggaagcaggtggggtctggcgtcaccacggaccaggtgcaggctgaggccaaagagtctgggcccacgacctacaaggtgaccagcacactgaccatcaaagagagcgactggctcagccagagcatgttcacctgccgcgtggatcacaggggcctgaccttccagcagaatgcgtcctccatgtgtggccccggtgagtgacctgtccccaggggcagcacccaccgacacacaggggtccactcgggtctggcattcgccaccccggatgcagccatctactccctgagccttggcttcccagagcggccaagggcaggggctcgggcggcaggacccctgggctcggcagaggcagttgctactctttgggtgggaaccatgcctccgcccacatccacacctgccccacctctgactcccttctcttgactccagatcaagacacagccatccgggtcttcgccatccccccatcctttgccagcatcttcctcaccaagtccaccaagttgacctgcctggtcacagacctgaccacctatgacagcgtgaccatctcctggacccgccagaatggcgaagctgtgaaaacccacaccaacatctccgagagccaccccaatgccactttcagcgccgtgggtgaggccagcatctgcgaggatgactggaattccggggagaggttcacgtgcaccgtgacccacacagacctgccctcgccactgaagcagaccatctcccggcccaagggtaggccccactcttgcccctcttcctgcactccctgggacctcccttggcctctggggcatggtggaaagcacccctcactcccccgttgtctgggcaactggggaaaaggggactcaaccccagcccacaggctggtccccccactgccccgccctcaccaccatctctgttcacaggggtggccctgcacaggcccgatgtctacttgctgccaccagcccgggagcagctgaacctgcgggagtcggccaccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcaccgagaggaccgtggacaagtccaccggtaaacccaccctgtacaacgtgtccctggtcatgtccgacacagctggcacctgctactgaccctgctggcctgcccacaggctcggggcggctggccgctctgtgtgtgcatgcaaactaaccgtgtcaacggggtgagatgttgcatcttataaaattagaaataaaaagatccattcaaaagatactggtcctgagtgcacgatgctctggcctactggggtggcggctgtgctgcacccaccctgcgcctcccctgcagaacaccttcctccacagcccccacccctgcctcacccacctgcgtgcctcagtggcttctagaaacccctgaattccctgcagctgctcacagcaggctgacctcagacttgccattcctcctactgcttccagaaagaaagctgaaagcaaggccacacgtatacaggcagcacacaggcatgtgtggatacacatggacagacacggacacacacaaacacatggacacacagagacgtgctaacccatgggcacacacatacacagacatggacccacacacaaacatatgtggacacacatgtacaaacatgcacaggcacacaaagagaacactgactacaggcacacacacacacgggcacactacccacaggcacacaacagatggacacgcgtacacagacatgcacacacccacaggcacaacacgtgcgcatgccggccggcccccgcccacattctcccagggccctgccggatactctgtccctgcagcagtttgctccctgcgctgtgctggccccggggctttgggcccaggctctgcttgtccttctgtctctgcttggaggtgctgccatggcacccagcttgggctctgcctggggagcggaggccccagggatagcatgtgacccctgctgaggccaggctcctgatgaaggcagcagatagcccccacacccaccggtgagcagaaccagagcctgtgccatgtgctgagagcaggcagtgactaagcatatgggcccagagggcagagtggctgccctgggcagctgctcctcttagcaggaggcctcaggagatgagctagagcaagtctgcccctgcaaataccacctgctccccaacccacagcagggagcaggcgaggtcagacagcagcagcccgggaaggaccgagccccagcagggaaggcagggcccgagtgaggtctccacacccaacgcacagtgctgtctctaactggggccacctccgagtccccgccacactcttggccctttggagtcctgggctccaggtgtctcccaagggcccatctgtgcaggggatgcaaccccccgaatgtcctcatcccactgtggagctcaggtctctgtctgctccctgggtcctggcagggtaggacaagtccgccaggatgtccccatgcagactctgctccaagagggagctggagagtcagggccttggtgagggagtcaggatcgggttccccccagctcagtcctcccacctgccagcccccacagcacagggcagggccacaccccctgcttccccctccaggagagtcaggacatgctggccgctgctccgctggggccccgccctccagcccccaccttggtctgtgtgctgcatcccccacgctctctctgccaccccaggactctgaggaaaagacctcagagtcccagccctgcccagcctcggcctgtgcccccgctgcatcaggctttcaggggcccagcccatgccctgggcagtgcccgagcccccctgcacttgctctccccacccctgggtgcagcacagcctaggggccaagggtgggcctagaggatgggccccgggggggctttgctgggtgccaccccagcctgaccctattcccccgtgctgtgtctcctgcagagggggaggtgagcgccgacgaggagggctttgagaacctgtgggccaccgcctccaccttcatcgtcctcttcctcctgagcctcttctacagtaccaccgtcaccttgttcaaggtagcacggctgtggcacagggaggagggcgcagggcgaatgtggggcccagggagcagcctgggctggacgtctagcccggaggcccccacaccaccccactgggtcatctctgccccggctcccttcccgaccacagggaaagcatttcacactgtctctgttgcctgtaggtgaaatgatcccaacagaagaacatcggagaccagagagaggaactcaaaggggcgctgcctccgggtctggggtcctggcctgcgtggcctgttggcacgtgtttctcttccccgcccggcctccagttgtgtgctctcacacaggcttccttctcgaccggcaggggctggctggcttgcaggccacgaggtgggctctaccccacactgctttgctgtgtatacgcttgttgccctgaaataaatatgcacattttatccatgaaactgctttctggtgagggtttttgtttctttttcaaaactttcatgctacatgggcatctcaagggggaccccagttccaaagggagctgtggaaaagagcgctgggtcagccagcgcagggggttcgaggaaagccacgtgcccaggaaaggggccgcagaagcaggtgggccagactcagactcggggcatgcccagcctgatggaaggaaggggactgagcaggagagggttccaggcctggtcctccaagcacagcctgaattgggagactggggctcaggcctcgggggcctctgtgtgtgctccacatgcctacaactgcccgggtcaccttgccacccttcccagcaagcccagacagttcttggccttgccccaaaccttcatgatgtgtggtgcacgccaccgggatccaggaggtgcaggctgagccctcgagagcatgtgggcctcaccgggatccaggaggtgcaggctgagccctcgagagcatgtgggcctgtctgcacagtgtgggggccttgcactccacaggagcacaggggtggggtagcagtcgcgcccgtggcaggggagtggaagttggagcaaaagtttcaggtgaacgagtgtcttagtctaattgggcagctatcacaaaacactatcggctgcaaagcttgagcaacagacatttgcctcccacagcgctggaggctgaaagtctaagatcaaggcgccggcaacttcaacgtctggcgagggccaattcccagctcagagacgcgcctcctcactgcatcctcgcgcagccgaagatggtgaaggagctctcagggtctctgtcatacaggcactaagcccgtcacgagggtctctgtcatacaggcactaagcccgtcacgagggtctctctcatacaggcactaagcccgtcacgagggtctctctcatacaggcactaagcccgtcacgagggtctctctcatacaggcactaagcccgtcacgagggtctctctcatacaggcactaagcccgtcacgagggtctctctcatacaggcactaagcccgtcacgagggtctctctcatacaggcactaagcccgtcacgagggtctctctcatacaggcactaagcccgtcacgagggtctctctcatacaggcactaagcccatcacgaggtcctcatcattgcccagatgctagggctcccagtgccatccccttgtggatgaggatggggggcgtgaccatgtcgtccttagaagccgttagggaatgcatagtggggaggacaagtctctgccatcaccctgaagatgggcaggtggtggccaggcttggggcagtgccgaaaaatgactccccaggtaggtgggagcaggatcaaagagcaggggattttttcagagatggcagccaagccaaagttgggggctcatgccaggaggctcacttggccatggtgcagtcacccaggggtgacccacagtccgtggcaaagtcaaggacagcagtggctgtgagaccagggtggaggcaggtggctggggggagaggaccaggctggaaggactgggcagaccccaaggccatagaagtgtcctagaggaagcatccaggatggcgtcttgggagggctcataagctgggggcccagaaggaggccatttcaggttctgaggagcagcctggcacttgctctgagccagctgtcatgggtatctgggggctgctgggtacaagttgtgtccctcccactaaatgtgctctccacatggacccggcccgcccttctgtccctgctggatccctgagctggcaccagccctgccctcagagacaatgtccaggagacaggtggaggtgcacgtgtgggtccctggggaaatccatcctccaaccgtgggctccgagtcgcctcccagcctctcgctccagcttcaccccatgtagctcatcacaggctcaacctcccagcctgggggaggatggagtgaggagccccacactgcccagggcacacccagggggctggggagtctgcactgggctggggcagggaggcctcgtgcagcctgtggggctggcagctcaggacaacactcgtatccgttaactgtggccctggcaacactgcaccccagactgcgtggcttaaacaacagacgtttattccgtcctggttctggaggccgggcatctgggatggaggcctcggcggggctggctcctctgtgtcacgggagactctgttccaggctctctccctgctgctgggctttgccggccgtctctggtgctcttggcttatggaagcagcaccatcttcacagggcgttctccccacgtgctgtctgtgcccagattcccccttttcatgaggacagcagtcatattggatcagaggcttgccctactccagggtgacctcatctgaacttgattgcagctgcaaagactgtttccagacaaggtcacattctgtggtcctgggggttaggacttcgacacattaatttacaggggacacattttaacccatgacagtttgccctccgttccccccataatcatgtccttctcacatgcaaaatccctgcatcccatagcaacatctccagaacggctgaccccttccagcaccaactcttagtccacaatctcagcgaaatatcacctgcatcaagtgtgggagaaacccagggtgagattcattctgggtgaaattcctctccatctgtggacctttgtaacttgacaagaagttatctgcttccaaagtacaatgatggggcaggcataggatggacattcctattctaaaagggatacatagaaagggagagaggagccatgggtcccaagcaagcccaaacccagcagggaaagctgcattagatttaaggctcaaggctcatcctcctgggtccgtgctccatcttccaggcccactggggtgaccccatgctcttcgcccatggtggccagagcccccacagcctcctcatctttggctctgatctcaaagtcacccttccttcattttttctggtcttctttcccttccatccaattggcattgtttcttctgttttacaattcacaaaatccttgtcagcttcccatgaaattcatgggggtttgagtcatcagacaagagagccctgcacagttatatcctcaataaccttatctctactcctggtttctgctgagatggttgattggatccataagtcatgattccaatctctttaacaaatggttgcccagacacatccttggccttgtttccagagcatgctattggacaggctgagaattttccagctgttcaagttgtgggtcctgtttactgaacactttctctcactttttactataagcagtcaggagaaatcaggttgtgccttcaacacttttcttagaaatatcctcagctaaaatccaggttcatcacttgtaagtcctacttaccataaatagaatgcaattcagccaagtgctttgccactttctaacaaaggtcacctttcctccaattttcaataacatcttcctcatttctgtctggggcctcaccagaggcatctttaacattcacatttctagcaacattctgtttgtgacaatttatgtattctctaagatgacaggagttttctctatagctctcctctttctttctgagcccccaccaggatcaccttcaatgtccaaatttctaccatagtcttcaaggaaatctaggctttttctaacatgcacctgaaaactcttctcacctctacacattaccctatcccaaattcatttccacagttttaggtatcaattaaatcagataaataaatcattaccctgaattatttcagttaagcatgttagttggtggcataagagaaaactcagtcagatagtgctgaagacaggactgtggagacaccttagaaggacagattctgttcagaatcactgatgcggcgtcagcaggactggcctagtggaggctctgggagggtggctgccaggcccggcctgggctttgggtctccccggactacctggagctgggatgcgtggcttctgccgccgggccgactggctgctcaggccccagcccttgttaatggacttggaggaatgattccatgccaaagcttttgcaaggttcgcagtgaccaggcacccgacatggtaagagacaggcagccgccgctgctgcattcacttctcttaaaactttgtatttgatgtcttatttccactagaaggcgaattggtcttaattgcttctcttaagccaccacttctaaagccaaaatctgttttagtttcttatgagctattgtaacaaagtaccacaactgcatggctgaaacaacagaagtgtattctgtctcatttctggaggccaggtgtctgagatcaagctgtgggcagggttggctcccctgggttggcagggagaatctgttccaggtgtctgtcctggcttttgttggtttgctggaaagctttggtgttccttggcttttggaaacagctcctccgcctccatctttaccttcacatggcattctgcctgcgtgcatgtctgtgtctaaatttccactttgcataaggacaccagccatattgggtcagaggctcacctgcctccagcgtgacctcatcttaacttgattgcatctgcaaaactctgcttccaaagaagggcacattctgaggccctacgggtcagagcttcaacacgtccatttatttggagacacaactcaacccataacaagacccatccctagtgtctcaccagtggtgctgtagagggaggcctggcagggctgacatcccagaacctcagggaaaaatgaaaacagcccagaggggtccaagaggtggctcgaggaagggaattaggaacacactgttcaggtggaagattctgagatgggccgtgaatactgcctaggatgtggagggttgacaatgggttgacaatcctgctcagaaaatcccaggataaaggatgtaaatggggagaagaggagggtcatccagaatttgggaaagcagggcgacagtttctgccccaagggagaagggaaggaggatggggccaccgccacaccagatgaccttgcgtaccaggccaaagaacgggaacacctggccccacctgagcagcaatagtcagtgtggtggtgggcagacatgggtggaggcagggggtgagtagaaggttagactaagacggagcacctggggcctccagggacccaggcaagaaccctgcacttgctcagctgccctgggtacccaggtctccaggaaagtgaggctgagagccaagcccagcaggcagccacacattctgggacctgccaccctacagcctgctgtccatgagtaacaccccttacaaggggccaggtcagctcatgggtttatcccaggcagcagagcccctggggccaggaatcagggagaggagcatccaatcccaccagctcctctggagccactcagagggccagacgcatggccctccacagggaccccatggcccctgcagggcagctgaggacccgtggctgggagcctgggcaggagggtcatacagccctaggcccgttgctcccaggcttgagtgcccctcctcccctcaggggcccaagggaagtgggttccagagaggttgggggcagcagggaaggtggaggtcccaggaatgcccagaggggcaccaaagcctctggagggaagacccctcccttccaggagctctcggcaacaagagcccagggtccacaaagccacaggtcccactcggttattctgactcacaacacaggagcggcagcaggggcattcgtgttcacgggccacttggtcagccccgctcaccctgggcactcctcctgggccccttttccctgccttccctgtcaccctgctgccagggtcctctgccctgccctgccccttgtcctcagagcctccagcctcagactcccactgtgtctgtcttccagcacccaccaaggctccggatgtgttccccatcatatcagggtgcagacacccaaaggataacagccctgtggtcctggcatgcttgataactgggtaccacccaacgtccgtgactgtcacctggtacatggggacacagagccagccccagagaaccttccctgagatacaaagacgggacagctactacatgacaagcagccagctctccacccccctccagcagtggcgccaaggcgagtacaaatgcgtggtccagcacaccgccagcaagagtaagaaggagatcttccgctggccaggtaggtcgcaccggagatcacccagaagggccccccaggacccccagcaccttccactcagggcctgaccacaaagacagaagcaagggctgggctgtgaggcaacccccacctccccctcagagcacgttcctcccccttcaccctgtatccacccctccggaccctccccatctcagtccctccgctccctctctctgaggcccatctcccaatacccagatcactttccttccagacccttccctcagtgtgcacggaggcagcttgcccagcaaaggtgactgtctagtgggcttcccacagccaagctcccaccccatgctgcggcccttcccttcttcctgcttggctgcctgtgccccccacctgcctgtccacaacccagcctctggtacatccatgccctctgccctcagcctcacctgcacttttccttggatttcagagtctccaaaggcacaggcctcctcagtgcccactgcacaaccccaagcagagggcagcctcgccaaggcaaccacagccccagccaccacccgtaacacaggtgagaagccccttccctgcacactccacccccacccacctgctcattcctcagccgcctcctccaggcagcccttcataactccttgtctgagtctccaagtcacactttggtaaggagagggacactgaacggacctctaacaaacacctactgccagccagccccagtctgggggccagcagatgccaaacagccagcagactcccagagcagacctgggccggctccctggcccatggacccagctctgcctcgctgagctgaggcatgggctctcagcgcagcctcacatagagccaccctgccgaggcagtccggcttgcagactcacaggtcacttgggccgcagcagcccctccccgtgaccctcgcctcccgcccgccccagcctggctctctccaagtgttggatcttggtggccagcctgcttctcaccctcaccctgcctgccacctcagaatggcaggggaaagagggccctcaccaagaactttatctgaggagtctgaggcttgtgactctgacctgcctgagatgtccatgtggccggggggacgggttcagtgttcgggagaactcgggtacgtgcctgactttctctgagtagggcaggaagctgttaggagaagcagcagtgaggtgggctggaccaacaggcagaatgactgtccctcagccaccctctgggatgtgggtcaagctctgacaaaggcatggcacagccatggtggcccctgcttggatgagtggccacggtgccctcaccctgggccagaatctgcctccactctgcaggtgcagaaacacgacattcccgtctctaaacacacctagctcctaggcttggggtgggcctatcaaatgcagggagatggacacagcacaagggccagagcttcccatgagaaaggtgagggcagctgctccctgacccgggcatctgcacttgtccctctccaccctcctcatgggcagtggagactcagcaacaaaacaagttgagtgcattagcagccagctctggagccaagtcactcaccccacggccttggctgctggtggaggggccttcccctgggcagcctccaagaagacggccaagtgctcttactcagaccacggcgctgcttcctggcacctcgatttcccacaacaacatggggtgcagacaggctagggccccctgccctggggcctggacggcatccagttaaagatgacccttcacgggcggtgcctgaggtgtgctgacctcagcagctaagccctcaggtctggtctgcactgccccacctggaggacccaactgacccagacacagccagggttatggcatgaccccgtggacggtgacccacaggccagatgcagccaggggctgttttgtgtggcctagaaatgtctttacagttgtagtgggatggaggaggaagaggaagagaggaggggagagaaaagcagggaaggggaaaaagaggagttcaatgcaaccccaaaagccagaacagttttgagctgaaagaacaaggcaggaaacatcccagtacctgacttcaaaacatactataaagcagttgtaatcaaaacaggatcataaaaacagacacacagacccatggaacagaaaagcgagcccagaaataaatctacatgcttgcagtccattgattttcaacaaaggcaccaggaaaacacaatggggagaggacagtttcctcaataaatagtgctggggaaactggatatccatgtgcagactaatgaaactacacaaaaatcaattgaaaacagtctaggccaggcgcggtggctcatgccggtaatcccagcactttgggaggccgagacaggcggatcacctgaggtcaggagttcgagaccagcttggccaacatggcgaaacccggtctccactaaaaatacaaaaattagcacatggtggcctacgtctgttatcccagcttttcaggaggctgaggcaggagaatcgcttgaatccgggaggtgaaggttgcagggagccaagattgcgccactgcattccagcctgggcaatggagcgagactgtctcaaaaaaaaaaaaaaaaagaaaagaaaacagtctaaaggtttaactgaacagataaagctactagaagaaaacatagggggaaaactccatgacattagtctgagcaacgatttttggatatgatcccaaaagctcaggcagcactagtcacaaaagccaagatacagaaccaacctaagcacccctcagcagatgcacaggtaaagaaaatgtggtacgtatggggcacaatggaatacgattcagcctttaaaaacagtgaaattctgtcattggcaacaatgtagatgaacctgaaggacacttatgctaagtgaaataagccaggcacagaaggagcaatactgcatgattgcacttacatctggcaggttaaaaaggcaaactcttagaggcagacagtagagaggtggtgccagggagcgggcactggtggctggggagatgttggtcaaagggcacaaaactgcagttgggaggaattagttcaggacatcccttgtacatggggacagtggttagtaacaacggattgtatccttgaaaaccgctaagaaaatagtttttaagtgttcttgacacaaaaagtgacacgtatgtgagatactgcatggtcattagctggatttagccattccacaatgtacacatatttcaaacattgtgttgtatatgataaacatgtataatttttgtcaattaaaaatttttaggaagaggaggagaagagaagaagaaggagaaggagaaagaggaacaagaagagagagagacaaagacaccaggttttttctgacccctgggctatcaaaacacctattgcccaataactagttggccgttggtgccctaaactattgaagcgattgctgttatgtggatgggccccggacacttagaaactcgtgacccctgaggacccccacgaggacagtcagggtccccccgaactcagggagcactgaggaaggagctcttagaggcgtggggcccctcaggcccctcagagggctctgccacatgggtcaggggcaggctgagggggagtcccaggctccatgcccagcctctgtgcctctgaccagggtgtcccccacaccgcctcctccccagtgccctccactggccacacctggccagaagctggggagaggagagcacagtggttaagtcagtccctgcagggagacggcaccagaaaaacctggcctgtggatgagtcccggcctggcagccacagagcagagagctctagaagcaacgaaggcccgagtctgctcagggaagagcgggcagcagccccagggccggacagtgaccaagagtggcaccgcccatggctcaacgggtctttgcccacagatcccccagcccctggagacagggtctgtgtgcctggccgtgcaggcaggcaccacactcagggggaggccactgtggagctctgtgcagagccccgggcgggagcctactgctcccgaaggtccggccacagctgctctcgtttgctctcccctgcagagtgtccgagccacacccagcctcttggcgtctacctgctaacccctgcagtgcaggacctgtggctccgggacaaagccaccttcacctgcttcgtggtgggcagtgacctgaaggatgctcacctgacctgggaggtggccgggaaggtccccacagggggcgtggaggaagggctgctggagcggcacagcaacggctcccagagccagcacagccgtctgaccctgcccaggtccttgtggaacgcggggacctccgtcacctgcacactgaaccatcccagcctcccaccccagaggttgatggcgctgagagaacccggtgagcctggctcccaggtggggagacgagggtgcccacagcctgctgacccctacgcctgccccagggccatgaccccagctgggccccagcagcaccggtcatcctccacaggaaaggagaagggaggcaccagcaccctggccggccccacttctctcccagtgcccccgtggccagaggctgacagcctcccccacctccccgcagctgcgcaggcacccgtcaagctttccctgaacctgctggcctcgtctgaccctcccgaggcggcctcgtggctcctgtgtgaggtgtctggcttctcgccccccaacatcctcctgatgtggctggaggaccagcgtgaggtgaacacttctgggtttgcccccgcacgcccccctccacagcccgggagcaccacgttctgggcctggagtgtgctgcgtgtcccagccccgcccagccctcagccagccacctacacgtgtgtggtcagccacgaggactcccggactctgctcaacgccagccggagcctagaagtcagctgtgagtcacccccaggcccagggttgggacggggactctgaggggggccataaggagctggaatccatactaggcaggggtgggcactgggcaggggcggggctaggctgtcctgggcacacaggccccttctcggtgtccggcaggagcacagacttcccagtactcctgggccatggatgtcccagcgtccatccttgctgtccacaccacgtgctggcccaggctggctggcacagtgtaagaggtggatacaacccctcgccgtgccctgaggagtggcggtttcctcccaagacattccccacggctgggtgctgggcacaggccttccctggtgtgaccgtgaatgtggtcaccctgaacagctgccctctctggggacatctgactgtccaagaccacagtcagcacctctgggagccagaggggtctccagagacccccagatgtcaggcttgggctcagtgcccagcgaaaggtcagccccacacatgcccataatgggcgcccacccagagtgacagcccccagcctcctgccaggcccacccttttccgcccccttgaggcatggcacacagaccagtgcgcccactgcccgagcatggccccagtgggatgtggtggccacgaggggctgtacacacagcaggaggctgtccgccctgctcagggcctgctgcctatgccccagctgtccaaccaagggaggcatggaagggcccctggtgtaagctggagccaggcacccaggcccccggccaccctgcagagccaaggaaaggaagacacccaagtcaacaaggggcagggctgagggctgtcccaggctcttttggcccgaggggctgccagcagccctgacccggcatgggccttccccaaaagcgaccctgtgaggtggcctcacagagaaccccctctgaggacagtgtctgaccctgcctgcctcacacagatgggccccacagcagtgggcaacctggggggcagcagcccaacctgaccctgcagggactgccccctgcagcagcagctgcttctcagtcccccaacctccctgtccccgccagagggtcttccccgaagctgcagccccaacccatggctgcccacctggaaccgggactccctgtccactgccccctccccttcggggccccatctgtgctggggcccaggttcggcctacagattcccatcattgccatggcctcctgaccttgcctatccacccccaaccaccggctccatgctgaccctcccccaggctcccacgcccagctggccggccatccccaggcacagacagtctgggatctcacaggttagcctggaccatccacctggccagacctgggagaggctggaagctgccctgccaccatgctccagggccccaggttgcagtactatggggtgagggtgtgtgtgcacacctgtgtgtacctaggatatccgagtgtacccttgtgcccccaagcacaagtctccctcccaggcagtgaggcccagatggtgcagtggttagagctgaggcttatcccacagagaaccctggcgccttggtcaaggaagcccctatgcctttcttgcctcgatttcccctcttgtctgctgagccagcaggggccacgtcctgggctgctgtgaggaggaagcaagttggtgctaggaggggctcctgtgtgtgcatgggcgggaggggtgcaggtatctgagcaccccggtctccacttgagagagcagggcaggagctccctgacccacccagactacacacgctgtgtccacgtgtctcccattatctgtggcagaggatccggcttctttctcaatttccagttcttcacaaagcaatgcctttgtaaaatgcaataagaaatactagaaaaatgatatgaacagaaagacacgccgattttttgttattagatgtaacagaccatggccccatgaaatgatcccggaccagatccgtccacacccgccactcagcagctctggccgagctcacagtacaaccacaataaactcttgttgaatgaactctaggaagtctgtgacgtggctggttcttgtcaatgcttcctgcctgcccacaggctcttcctcgtggatggggctgtgcttgccacggaagcgcgtttttcccggcctaggcttgccttgggccccactgccgtctccagctggagatgaccttctatacacacatttgctcatgacagacccttgcttagcccccttccatggctccctcctgctgctgggataaaatcaccttgcctggatatcccctcctgggcccctttccaccctccttagtcagcacccccagttcagggcacctgctttccccgctgcggagaagccactctctccttgctgcccggctgtgtcttgccttccacaccttgtcacagtggccacttcctaaggaaggcctccctgtgtgcaggtgtgcagaagtgccccagcctcccgtcacctttgtcacgggagcccaatccatgagagtctatggttctgtctgtctgccccactcagggcagcgacaagtccaggcggggaggacacagtaggcagagatttgtcgaggggacatatgagcaagagggtgaggctgggagctccctggagataaccacgcctcctgggaagactcgccgtcatttcagctccacgctgtgcgggggtgggtggaggggtagcctggccctcatgaccagggagcttctcactcagcccctgttcctccccagacctggccatgacccccctgatccctcagagcaaggatgagaacagcgatgactacacgacctttgatgatgtgggcagcctgtggaccgccctgtccacgtttgtggccctcttcatcctcaccctcctctacagcggcattgtcactttcatcaaggtcaggggagcggccaggctctcagtgaccctcggggtgggtgtggggcaaggtgcccttccaggggacatgccagagctggtccagggatcctggaccaggcagaggcagggctgagggagcctggaggacatgcaggccctctgtggcctgtggacactgtcgaaggccctcttgaccctgtggataaaggacaacaccccctcccctgctcctctgtctcccctgcccctccacccctcaggcttctagccccctgtctgaccccaggggctgtctttcaggtgaagtagccccagaagagcaggacgccctgtacctgcagagaagggaagcagcctctgtacctcatctgtggctaccagagagcagaaaggacccaccctggactcttctgtgtgcaggaagatgcgccagcccctgcccccggctcccctctgtccgccacagaatccagtcttctagaccagggggacgggcacccatcactccgcaggcgaatcagagcccccctgccccggccctaacccctgtgcctccttcccgtgcttcccccagagccagctacacccctgccccggccctaacccccatgcctccttcctgtgcttcccccagagccagctagtcccacctgcagcccgctggcctccccataaacacgctttggttcatttcacctgcctcctgttctttgtcccagtggtctggattcacctcaagttaaaggacacagggagctaacggcaacggggagggagtttggggtgtcagcagcaacagggaacgcaatagccaaagctgtcacagcaagaactgagcggagaccttgctctgaagttcatgggttaagaaacgcccctccccacaccaaacacaaacatttcacgctcactcgtacatgcacacacatgcacacaagcatctatgcacacatgcacatgtgcacagctcacgaataaacatatatgcacacacatgcagtacactcacatgcaaacaggcagatgcacaggcatgggcacacatgcacaaggcatgcatgtgcaggtctgcagacacacacatctgcatgcacatacacggacgtgcacctgcacacacacaagtgcacataggcatcaaagacacacatgctcgtgcacgcgcatacacacagacatgcacacttatgcatgcacacgccatgacaggatggcacgggaacaaccttgcacatggagcctgttctgtgtgtatcgagagcctgcacctcttactgtctgcatgactgtgggcaagagcctgaactccgtacctccatttcctcatcagaatggggtgaaaatacacctgtcaagtagagtgaggtgagaagtgagtaaaatcctatctgaaaggggctcagcaaggacccaggtattggtcggacctcctgttatcatctgctgtgggctgaacatttgtgtcctcctgaaattcatacattcgtatattcatacctttccccctgaaatcctaacctccaatgtaatggtattagaagatggggcttttgggggtgctgaggtcataaaaggggggccctcatgaagaggattagcacccttatgagaggggccccagagagctccctcaccctttctcccctgtgaggagatgccgtctatgagctgggatgcaggcctcaccagacacccatctgctggagccttggtctgggacattcagcctccagaatggtgggaaataagtttctgttgtttctcagccaccacgtctgtagtatgtggaagtcatcagaatcaaaattgagtcacctgtggttttttttttttctaaatccctgacaaatagagcctaggaaggccaagaagagaagagggttctcatccataaacacttgataacaaaaactatcaccaaggactctacaaaaactgcaactggcacaaagaccatcacaaccttacacagaaagtacttctgtgaggacatcttcccagcaacgggctgtccaacctcagactggcattgcctttgttattggtccttgtagagagggtaattatctcaaagcaatcatgtaatcctcctcatttttcctttgaaagccttggtctccctttgcctccctgaatacgcacatagctgatcatggcaggtgtatcccactgcagtgctctacctccaaatagatatctttttcttttagacggcatttctcttgttatttagattgagatacatggagtcagaagtgggatgtggagaaggatcactgctggaaggagtcagtaattcttgggccggtgtgcagtattcacttgagccctttgagctctcagctttttctgagctactccgtcttctctcaggccaaccctccctctttttggaagatgcttttttaatattatgtggggtttgtttattagactgccttaataaaggaccttatgtccctcctgggatgataaaaggcttttcgtgctttctggcaagtcctgtttagcataaagacattccagcctgagtactctggtttccacagagtttgcattctgtctctgagggatgtcttctctggtgaggtgcctcctttaatactttgttggatatgcatgcctgggtgaacatatttgtgagcacccttatcttgccttcttttgctgtggttatgtgtatctgtaaatgacctggctcttttcctctgcttgcttctaaaaatctttggagagcaaaataaacattctaaatggtgggcacaggctgtctcattagaagctgctacagcagtcaccaccgtctaaagcaccagtccaaactgccggcgtggcctcatagtatctgtagaatgttctttactgtaaaaagattaataagaagggggatggtttttaaatattaaggcacagtaagttttctggaactcccgctggctacgtctttgtgcacatttttaaactaatgggcaaattacgtcaaggaaaatttagagttccaatggttatttttcgaactctaacaaaaaccctgcaacagtagtcatcatgcagtgcctcctaagtcctctatctctctattttgtttttctgcctactttaaatctgctgacttttttactcatgttgcaataaaactcactggttacagcattccagccaagatatttttggttttttgtttgtttgttttttgttttttgtttttttagatggagtctcgctctgtcacccaggctggaatgcagtggcgctatcttgggtcgctgcaacctctgcctctcgggttcaagtgattctcctgcctcagcctcctgagtagctgattacaggtgcccgccaccatgcccagctaatttttgtattttagtagagacaggctttcactgtgttggccaagctcgtcttgaactcctgacctcaagtgatccacctgccttggcctcccaaagtgctaaaactataggcgtgtgccaccgtgcctggcccaagattttttaaagtttcctaaaggttttaaattagtggctttacaaattacaacagctctatggtaaccaatgacctagatgtaagagtcatttcttttgaaaatgtagattagctttgcttggctaacaactgcttaggctgatggaatagctaactgaaggactgatggtctcaaagaacagaactaggtaaatatttacagaaattgggctttcagatcgaacaggccaaaatcttgagcttagagcagtaatataaggtatctctgtctgccataaaaattttgctttgccacaggtgccagaaaaaggaaaaacactgctaaatgcttccctgcatgcattgtctagtccagaaaatcagaccagcaaccaaaaaatagatttgttactagtatagacgagttgaagattttgtttttcatatacaattcagccagtcctagctaaaacgcaatcactggaaatttaaccttaaattcatttaaaactgaaaaaggtcttttgaaatcaaactaatgcagaaactgctttacccaaaattctgatccatggccctcattagacgacccatcaggacaaataaagtttagcttgtgaacaagtcccagttttgtcaaaaatataatttggattgaagtgtcttttagaaactgacacatttgtgttattatctcatgaccaaaattctaaaatgaaagctacagtgtctttatttgtgtgcgtatgtggttaagtgtgtttatgcatatgtatgtgtattatgttgtatgttgttatctacatggtaaaatctggcatcatcaacaaaaaatctattaagggattctatttagattggcttagataaataagcattcataaaaaatatgtactaattaacccaaatgccttttagttcatgtgacttaggtatatctttaataaacaaatcagttttaaaattgttggtaaaataaaaatacaaatgttctcagaattgtcagcatatatttttccctgagtttactcgtcagacagctttatatttgtctctgatagatgttttaagatgtcagggtttgacacaaagatgataaaactataaacccatcctaaaacagaataatctttgtatgatctttgataaataagactaatttggctgagtgcagtggttcatgcctgtaattccagcactttgggaggccaaggtgggcagatgactagaggtcaggactttgagaccagcctggccaacacagcaaaaccccatccctactaaaagtacaaaattagccaggtgtggtggtgggcataatcccagctacttgggagcctgaggcaggaaagtcactggaacccgggaggcagaggttgcaatgagccaagagagcgccactgcactccagcctaggcaacagcgagactccatctcaaaaaaaaaaaaaaaggctaatttaatattgcaggcttaataaaaacagctgtatcttatgagctatcgacaaaatacccatacacttcactaagtttcttactaagtgaacatctaataatcacaggctataaaaatggttaaaagggaaataactttaagtaatggctagctttgtctaatatctcaattttcataagtaatctaggtaaattattaaaaataaattaattaagcctggtgtcatggcacacgcctatagtcctagctactcaggaggctgaggtgggaggattgcttgagcccggaagttcagaccaccctgagcgatgcaacaagacccccatcactaaaataaataaataaataagataaatgtcaagaaaataaatgtttataaataagcttcttatgtaatttaagatcttaaaattatgttatcttcagttaaataatagatactcattaaatgtctgggtcatttccaaataagatttttaaaactaacacaaattactgaacataaatgtttgttcttggcttcttaaattttatagaaattttgtagaaagactaaatttatttgggtctattaatatatgtaaaaattatgtcatagaaatatgttttcaaaaattataaaatttttctcatctataaaatagtgatatgtgacaaatggttaaaatttgcttgctagaaaataaggttactaagagttaaaattctaattaatatatataattctgtctacaaagtataccacaaaaaataagatgtgtttttcatttttaaaaactataagacaggcatttataatttattttactgcaaaaaaaaatttatctaatttggagtttgtttaaaggttgtttcaaagcacagatttaggaaaaaagtaaaaacaagatggaaaagaaccactctcatctgcccccacgtaagacgtacctgctccccttccaccacgactgtgtttcccaaggcctctccagccatgtggaactgaatttaatctacaaatagagttgatcaagtaactttaaggtatatggacaataatatgctttgcagcataaagacctgaaaacaaactaaatgcccatcagtgggaaattgtttcaataaaccacagaacattccgtggaaaactccacagccattcaaaagaatgaagggggagagatcatcatggtggacaggagtcaggactagattgcagtccgactgggatggacagagcacatgtggaggctggcaccgtgaattttagctccagaacaactgcaggaataaatcaggaatcccaagaggatccacagaccatgtgaaggaagcagcctgctcctgcaggacccagagacaccccaaatactgtgagtgcccaaactgtggaagtgggaatgggagatcgtccatcctgcacacacaccctcactagagaaaccaaaggtctagttggtgggaaaaggttccaaccttacctggagctgagtcaatttagaaagctgagcgaaatacaggggtacagggagcagcgggaaaggccctgggagcttcctgggtccccaggcaggccattcctgcatggcaccatatgaatactttgggagggtggccagaggcacagggaaaatgtcacagggagaaggaagtctccagctgaacgcaggggagggcacgaatcctgcagaccccactggtagaggaacaaccaaagcccttcttcatagctgggaagtaggtagcctggggcaagttctctgcttgcccaatgcctggaaacagactcagtgctgttggcgaggggcatggtgtgagtgagacccaccttcagtttgcatgggagctgggtgacacctgtgactgccggctttccccacttccctgacaacctgcatgactcagcagaggcagtcataatcctcctaggttcacaactccattgacctgggaacctcacagccatcccccccagcagctgcagcaagaactgtccaaggagactctgtgagctcagacacacctagccctgcccgcatctgatggtccttccctacccccccttgtagttgaagacagagggcatatactcttgggagttctagggccctacccactgctggttcctctccatactaccaccactgatgctctctggaaaacgccatctcctggcaggaggccaaccagcacaaaaacagagcattaaaccaccaaagctaagaaccttcatggagtccatttcaccgccccaccacctccactgggacagttgctggtatgcacggcggagaaacccacagacagttcacatcacaggactctgtgcagacaacccccagtacaagcctggagcctggtagacttgctgggtggctagatccagaagagaaataacaatcaccacagctcggctctcaggaagccacatccacaggaaaagggggagagtgctacatcaagggaacaccccgtgggacaaaggaatctgagcaacagccttcagccccagaccttccctcagacagagcctactcaaatgagaaggaaccagaaaaccagctctggtaatatgacaaaacaaagctctttaacacccccaaaaaatcgcactagctcaccagcaatggatccaaaccaagaagaaatccctgatttacctgaaaaagaattcaggaggttggttattaagctaatcatggaggcaccagagaaaggcaaagcccgatgcaaggaaatccaaaaaaagatacaagaagtgaagggagaaatgttcaaggaaatagatagcataaagaaaaacaataaaaacttcaggaaacattggacacacttatagaaatgcaaaatgctctggaaagtctcagcaacagaactgaataagtagaagaaagaaattcagagctcgaagacaaggtctttgaattaacccaatccaacaaagacaaagaaaaaagaataagaaaatatgagcaaagcctccaagaagtctaggattatgttaaatgaacaaacctaagataattggtgtccctgaggacgaagagaaatctaaaagtttggaaaacatgtttgggggaacaattgagggaagcttccccagccttgccagagacatagacatctaaatacaagaggcacaaagaacacctgggaaattcatcacaaaaagatctttaaaactaaaactaaaacaagcaatctacaaaacctaggcacattgtcatctggttatctaaagttaagatgaaggaaagaatcttaagagctgtgagacaaaagcaccacatgcttagtttcactggatacaaaattcttggctgataattgttctgttggaggaggatgaagatagggctccaatctcttctagcttgtagtgtttctgctgagaaatctgctgttaatctgataggttttcgtttataggttacctggtgctttcggttcaaaaatttttttaatttccatcttgatttcgtttttgacccaataatcattcaagagcagtttatttaatttccatgtatttgcatggttttgaaggttccttttggagttgatttccagttttattccactgtggtctgagagagtgcttgatataatttcaattttcttaaatttattgaggcttgctttgtggcctatcatacggtctgtcttggagaatgttccatacgccgttgaatagaatgtgaccatatgataggaaataaactccaaaagaagccgaacaacaacagtgacacaacctgttgaaacctctgggatacagcaaaggcggtgccaagaggaaagttcacacccctaggcgcctacgtcaaaaagactgaaaaagcacaaattgacattctaaggtcacccctcagggaaccagagaaacaagaacaaaccaaatccaaacccagcagaagaaaggaattaatcaagatcagagcaaaactaaaggaaattgagacaaaaaaatacaaaagataaatgaaacaaaaagctggttctctgaaaagataaataaaattgatagaccattagcaagattaaccaagaaaagaagaaagtcccaataacctcaataggaaacgaaatgggaaatattacaactgacaccacagaaatacaaaagatcattcaaggctactatgaacgcctttacacacattaactagaaaacctagaagagatggataaattctcaggaaaatacaaccctcctagcttaaatcaggaagaattatataccctgaagagaccaataacaagcagcgagattgaaatggtaattttaaaattaccaagaacaaaaaaaagtccaggaccagattcacagtagaattctaccagacattcaaagaagaattggtcctattggtactattccacaagacagagaaagcgggaagcctccctaattcattctatgaagccagcatcaccctaataccaaaaccgggaaaggacgtaaccaaaaaagaaaactacagaccgatatccctgatgaagatagatgccagaaatccttaacaaaatgctagctaaccaagtccaacaacatatcaaaaagataatccaccccaatcaagtgggtttcataccagagatgcagggatggtttaacatacgcaagtcagttaatgtgatacaccacataaacagaattaaaaacaaaaatcacatgatcatctcaatagatgcagaaacagcatttgacaaaatccagcatcgctttatgattaaaactctcagcaaatcagcataccaagggacatacctcagtgtagaaaaagccatccatgacaacataattctgaatggggaaaagttgaaagcattccctctgagaattgaaacaagacaaggatgcccactgtcaccactcctcttcaacacagtattggaagtcctagccagagcaatcagacaagggaaagaaataaagggcatccatatcgataaagagaaagtcaaactgtcactactgatgatatgattgtttacctggaaaaccctaaagactcctccagaaagctcctaggactgaaaaaaaaattcagcaaagtttccagatagaagattaatgtacacaaatcagtagctactctatacaccaacagtaattaagcagagaatcaaatcaagaactcaaccccttttacaatagctgcaaaaaataaaataaaatacttagaaatataccttaccaaggaagggaaagacctctacaaggaaaactacaaaacgctgctgaaagaaatcatagatgacacaaacaaatggaaacacataccacgctcatggatgggtagaatcaatattgtgaaaattaccacactgccaaaagcaatctacaaattcaatgcaatccccatcaaaataccatcatcattcttcacagaattagaaaaagcaattctaaaattcatatggaggccaggcatggtggctcatgcctgtaatcctagcactttgggaggccaaggcagtaggatcacttgaggtcaggagttcaagaccagcctggccaacatggtgaaaccccatctctactaaaaatacaaaaactagccgggtgtggtggcatgtgcctgtaattccagctactactcaggaggctgaggcaggagaatcacttgaacctggaaggtttgcagtgagcccagattgtgccattgtactccagcctgggcaaaagaatgagaatctgtcacaaaaaaaaattcatatgaaaccaaaaaagagcctacatagccaaggcaagactaagcaaaaagaacaaatctggaggcaacacacggcctgattccaaactctactataaggccatagtcaccaaaacagcatggtactggtataaaaataggcacatagaccaatgtaacagaatagagaacccagaaataaacccaagtacttacagccaactgatctttgacaaagcaaacaaaaacgtaaagtggggaaaggacaccctattcaacaaatggtgctgggataactggcaagccacgtgtaggagaatgaaactggatcttcatctctcatcttatacaaaaatcaactcaagatggatcaaagacttaaatctaagacctgaaactgtaaaaattctagaaactataaaaatttccaataacattggaaaaacccttccaggcattggcttaggcgaggacttcatgaccagaacccaaaagcaaatgcaataaaaacaaagataaatagctgggacataattaagctaaagagcttctgcacagcaaaaggaacagtcagcagagtaaacagacaacccacagagtgagagaaaatcttcacaatctgtacatctgacaaaggactaatatccagaatctacaacaaactcaaacaaatcagcaagaaaaaaacagacaatcccatcaaaaagtggggtaaggacatgaatagacaattctcaaaagaagatatgcaaatggccaacaaatatatgaaaaaatgctcaacatcactaatgattagggaaatgcaaatcaaaaccacaatgcgataccaccttacccctgcaagaatggccataatcaaaaaatcaaaaaacattagatgttggtgtggatgcagtgatcaggggacacttctacactgctggtgggaatgtaaactagtacagccgctatggaaaacagtgcagagattccttaaaggactaaaagtagaactactatttgatcctgcaatcccactactgggtatctacccagaggaaaagacgtcattctacgaaaaagatacttgcacactcatgtttgtagcagtacaattttcaactgcaaacttgtggaaccaacccaaatgcccatcaatcaacaagtggataaaggaactgtagtatgtatatgtgatggaatactactcagccacaaaaaggaatgaattaatagcattcacagcgacctggatgaggctggaggctattattctaagtgaagtaactcaggaatggaaaaccaaacatcgtatgttctcactgacatgtgagagctaagatatgaggatgcaaaggcataagaatgatacaatagactttgggaacttgggggtgagggtgagagaggaaagagggagaaaagactacaaatatggtgcagcatatgctgctcgggtgatgggtgcaccaaaatctcacaaatcaccactaaagaacttattcatgtaaccaaacaccacctgtattccaataacctatggaaaaataaacattaaaaaaattaagaagactgaaagaattaaaaaatttaaaaaacagtaagtagaagagaggtatttgaaggaagttatgggtatgaagatgtatttttggtacggaaggttaaaaagaaaagagaatttttttaaaaaaggaagaatcttgcgtgataaatttttgtcataaagtaaaatgactgcttactttaaaaaaagaggtatcacacacaccagggcctgttgtggggtggggagaggggggagggataggattaggagatatacgtaatgtaaatgacgagttaatgggtgcagcacaccaacatggcacatgtatacatatgtaacaaacctgcacgttgtgcacatgtaccctagaacttagagtataataaatatatatatatataaagaaaattccacacctgaagccatgtgacaggctacagacaaaacacagttaaaactttgtttcatgtacaaagttatttaaaatattgtacaaaatcaccttcaggctgtctgtataaggtgtacatgaaatacaaatgaattttgtgtttagacttgggtctatccaagatatctcattatatatatgcaaatatcccaaaatccaaaaaatataaaatctgaaacacttctggtcccaagcatttcagacaggggatcgttagcctgtaatgagagggagtatgggtcaattatgacgttgaagattcaaaaaaaatttttttttaattttcagtcaagctctaccaactacactaactagaaaacactcaggcagattgctttttgctaggttaatatggcatgattacaactgtgcatatgaataacatgtatcagattccttttgtttgggaattatttcattacaacactaaacactgcttatgtacgctgggtctctttttcaaagactacaaataatgcttggttctttgctttcaaaatataccaaacatgaggtacaagaatgtacccatccatatttgaccaggggtagaattaaaaggtttgggttgaagaagatgacataacatcccaagagggttgcatccctaggggtcatgggaacaaatgtggccaagtccctttttaactggaaaggatttcagatccctgaacaagacaattcatcagtcttaagcgtagtaagtcatcagcaaataagagaatgataaacactagaaaaataatcaaatcattgtctaaattcattataaaactatatgagaaaactataaaactatatgagagagataaacagagacaagaagacaaggataaaataaaaaaagaggtatcaaacaaagcagaacgcctcaacatgtcataaaacatctgagtaagtcataataaggtttgcaaataatgaatttacgaaaggaattttgcgtgtgatcaagatggctataattagaagggaattatttataagtctttctaaagactgaggtttgctattaaaaatatgctaatataaaactaaagatttagtttcctgtgttagaacaacaaagttatcttgaagtattgatctgttcttaaaactacaagaagtttttatttttaattctaaaatctgtttctttaacagaccatttctattgcttcctgggatccatttactttccctagtttcaggttggaagtcctcttcatgtaaaacgagaatttcatttcttgacatagtcttttccccctaaagcttctcagttttagatttcagaacttcaacttctgttgtatttcacagcacatgatttatagatcatgtatataaatatggatttatagatcatgtatataaatacagacttatccatcagtgccttcagctcttcctccccatgagatggcctggggtgatagctctctctttcaactttttttttatcaactcctataacattttttctcccattacaactctgttgttatggctcgatgctgaaatgtttatcccgaaagtctagaaaacaaatgttctctccagtataattccagtataattgaattttcccttgtaaccaggaagtttctcatgctgttgctttttctatgtgttcccctgctcaggtactagttatcttgtttacatttctctactaatggtttacacttatagccttggacatactctttctctgcctaattaaactcagtgtctttttcatcagatttgacttccagtttatctacatgggcttcccaagaggagacacaatcacactgcaggaggcatttctttaacttttgggtaagtagcctaaaaaaaacaaagattttgtattttattaggataattttttgtgttgtctttatgaggtttttggttacttaggtaacttgagctttgaagaagttaggtccttttaatccatgtaactttccgtattactcttcaagtcttttgatgatcactggttgaatacatggctatatttaatagtgacctgagattctgctttgattagccatgttgaacctttgacatctttggcaggcttcctcaggatcaaaattctaaaataagtctttttttatctagaattgacttagggattttacagttagacccctggaaagcctcaaagaatttatctctcatcctatagagaaaataaatgattaggcttatttggtaaattatatgggaaacattgtcaaataataagtgatattagatcttctttcagttacatttgtgggtatgctattgatatgatgtttcaaagattatataaattcatatcagtctataacgttatcagccataattttggttatgctacatcttctttaaagctatatttgtatggagacgttactgatgtgagtatattctaaagattatgtgaaatttataaaaccctgatggttctgatgtgatgctatcagtcacagtcatgattctgtttgctaccttaaaacactatagtaatttttaaaaagtcaatttccttatcaattgctgattataatgaatttttatcagatgtttaaccatggccattttgtttttgtgacccagagttattgttttgatttttctccaaaagcatttgtaatcagctattgtccaaaattgcttttcatggaagagactcaaacaggaactcttaaatacggtttcccgataaaagatcaatggactcaataaaaaattttcagaactctaataaagaaactgagaaattcaaaaacctccaatcaagctcaagcagaaaagctgacttcatgatattgaagaagtgatgaggggaatatttttatgaattttatttgaagcatcgttcgttcttaaatgttttgtttgccagatttaaggaaattttctctcgtaagtcatctatagtttacagtaatttaatacagtatactttttgtgaacaaagatgaaagcaattattttcccccctacgtgactcctccaaaatttagaaactattcacaagtgttcttatgacgatgtggccatttgtataagtccagataaaaactagttgtctcctcactgcaggatgtaattggaaacatcaggtatattactaaggctttggctgaaataccatatttgaaaaatatacatagaatgcctagtttcagccgggcggggtggctcacccctgtaatcgcagcactttggaaggccaagccaggcggatcacccgaggtcaggagttcgagaccagcctggccaacatggcaaaaccccatctcaatcccgtctctactaaaaatacaaaaattagccaggtgtggtggtgggcacctataatcccagctgctcgggagactcaggcaggagactcttttgaacatggaaggcagaggttgcagtgtgccaagatcacgccactgcactccagcctcggcaacaaagtgagactccgtctcaaaaaaagaaaaaagaatgcctggtttccagggttccttataatgagtaaaaatcatcacttcctggcaagcccagaaaccttaaaactgtaagtaaaagctaaagcctgtcttggtttggcttaccagcataaagaggtattgaagtacgagattcctatgtgatcaatgtacagaggaaaacttatgcttccaaagaaaagctgtaacacacctgcttttagattgtagctctgtgcattattttcaagttcttgttttctacctatagactagatcctgaattcttctagattattccaatccaactttcttccatggagaaaaatgagaactgctctgttcctgaagccctataagctgaagctagataaatgctaagaaacaagtctcatgcttgatgtccgagccagacagaaagttcaccagactgcccaatgccacgactagagacattcaaactgcaaagcaagatgaggaatttcacattttcactctgtagacagcttctcccaagtcatgggaacaagactccatatcataatgggactcttacctgtcctagggcctacagtttttacttagcttcctattttatttattcaattaattgccttgaatcctggattcattatttattcaattaattgcctttaatcctagacattgcataatcctattatgcaaactaggattgtcatgttattactaattttactttttattttccctttttaaactttgtatctgttacttgctgaattttttcagaagtacaacttctaacagaataatgctagcccagcactttgagataatagcaaaagaccacaccacagacaaaattgaacttaatcatttactccaggtagacttagcctgaaagccactgtcttcaaacctcctttgttactcaaatgtggctaaaaggattttgacactgactcctagacatcaatcactccttcaaacatgtgaccagaccagacacctgggacaggcccatccaaatactgagggacatcagaacctaccacagcatggtccatcagtgaggcttccagagaaagaccttgaccaaagggagaaaatatgatgaaagtcgtcagaatcaaaatggagtcccttgtgttaaaaaacaaacaaacaaacgaaactctgacatataaagccagagaaggctgagaagcattctcatgcataaacacctactagcaaaaactatcacaaaagactataaaaaacacagccttgcacaaaggccattgcaaccttacacaaaaaaatacttctgtgaggacagctgcccagcaactgcctgtgcgcctcatactggcatcacccttgttattgatccttgtagccaaagattatttcaaaacgatcatgtaatcgtcctcatttttcctttaaaaacctttgtcgtcttttacctccctgaatatacacatagtttatgatggtacgtgtgctcccactgcagtgcttggggaccaagctccctccacacctcactgttcccaaataaatattttctgttagagcctctctctgttcattatttagctcgacaggtatttggtacagcagccaaaacaggccaagacattgcccggaatcttccagcagtagactgggggcccggatttgaccctggtcagttggacacgggcttctctcactccaccagatgaggaacaaatagtgaggtgtggcgggagcttggtccccaaacagctcctccggccatttgccctccccacccacggaccagagacaggacgagggtgagccccggtgtcagccatgtgccccaaccccggtgcctcatttgctcctaggatgcgtcctgtaggtcatcttcttgcagctgtcccgctcagtctcccctcaacccctgctcatgctcttggtgagcttatcaatattatggtttccaatagcttctgttcatagtttcaactcatatctgtccctaagtgtggacttttattccacgcgttggcactggcagtcagctatgtgcctcctactcaaggcatctgaaactgcaggtgggtagttggctaagtcaggccccaaaagtgtcttcatccagattcccagaactgtgactctgagatcttacatggcaaaagggactttgcagatgagattaagccaatgatcttgagatgaggagaggggcctggattacttgggtggctccgaatgtgggcacaggtgtcacaggagggaggcagagggagacgtgagaaggagaaggtgccatgactccgaagccagaggctcctgctgatcctccggccccacccccaccccacggaccatgtggtctttttaaaacatgaatctatcaggacggcccattcaaacccacagagaggttcccactgcaccaagaacaaagtcagaactctcaggcctgacccactgccctcggccaccctggagccccagtgcccccagacccggtcctggcctcagagtgtaacaggctcgctcccaccacccatctgcatggcaggcgccttgccatcgcctcccctctgagagcacagacagccacagctggcctccgggtgaaaggatggatgaacagtcaggccgggcgagtgctctcagagcctgctggccaacggaggcagccgctcaggcctctgcctgagccatgtgcgatcacagctgctgcccagctgtcccaaggaggcagctcttggcttttcccctgggagtcgaattcgcagggagtcccatccacaggcccctgggaggcgggaagacatctcggtctccaaagccagtaacctgtgattcccctgaggatggtgggaacctggtgtggggcagatgatctgtatttgagctttgtttctctctagcaattatacctaggaaataccctaaaatgcacgacgcaatgcacattgaccccagcaaactccccaccctgcgcctgtttctagtgccggggctgctccagcacagcctgtgcacttgaccgaatgtctacagccctggccctcagccacactgtgttgttttcatgcaccttcacccaacagaattgctccaacagagtgattcagggggctgccctccagcaccacttcccctttccatgttacataaatgtccattgagtcctgactgactacagaaatctgtcttctgggggcatgtgatgccccaggtggcccagggccccgcacaggaatgtgtttacagctttcccggcactgaagccacagtcgtaactcagcctcctgacatgggccagggccctcgcctccgcacgctctgcagctcacccgtgtcgccacagcctgctgggcctggacgccaccctctgaggtggcactcggggcagaaacatctgtttccagctggtaacaaacaggacatgtctgcctcccacacctaaatcaatgcttgacaagcctggcctgatctcggggcgctcaccaccagccacctgcctgggcccagccgacctcctctcccttggtattttgagggaagggtctgagccctttctgcccccctagacttgtggacagaggccccccaagacctggacagaaatcaggccatagtctccagccccaaagagacttccagaggcctgcacctctcctctccagccgaggccctgatgtctgtggctgtggccacagccggctctatacctgccaagtttattctagaaaacattggtttggacaagacctcaaaacggaattgggaaaatgaaagagaaaagagaaaggatttctgctctcattgtaaggacaaaaagcaaattgaagaccagcactgatacagatcaataatcacaccagaaaaaaatatggcaaaatatctatattctcttaaggtgaataagtgttctctcagaatgaggctaaggccaggaacagtgaaaagtaagactgataaatttgcccacaaggaaaaaaataaagtctctataaggacaaagccaccaaaaacaaagtaagcaccccaggaaagaaagtgaccaaagtatctaacatgaaagagagcctgtattcgcatgagctcccgagaaacagaacccatagatgtggatgtggatatcgacagaaataggtatcaacatagacacaggctatggccacaggtatggatagagggagagatgcgagagagagattttaaggaattggctcacacagttgtggagacgaatgtccaaaatctgcagggcaaaccagcaggctggtgacccagagaagagctgaggctgctgctcccgtccaaagttcagcctgctggcagaattctgtcctccttggggactgccgtctgttttctcttaaggcctccaactgcctggatgaggcccacccacattatggatgatgatctgccttacccaaagtccacaggttgaaatgttaataacatctaaaaatcacctgcactatctagactggtgtttggccaaatatctgggtagtatggcctgcccaagttaacacctaaaattaactatcagagtccaagtaagtgaatgtaaaaaacaaacaaacaaaaaaaaaaaaacatgatgaatgagacaagctcgaccttgaagaagttttcagtggaacgtggggaagaggagttctctttagccagctgcagacacaaatagccaatagaacacatggagagtgttaaaagtcacaaatcgtcaatggaaagcaacttacaatgtgatgttcattgaacatctactgtgtgccaggccaagggctaaggtggacacgtgcagtgtgtctgcactgcggcactaaggtcctggtggggaatggataatacaattaaaaacaaatcacaaagaaaccagaagaggaaaaaaagcaattaaaaacaaataaatgctccataaaggacaacagcgccagctgagggtgtggccatcaatgccaggcatccagaatatgtccttggggacatggaccactgggaggtgcatcccaggcaaaaggagcaggaggggaagggtccaggccagaaacatgcttgccaagttcaagagcggtgggaaggccagtgggcttgggaaggagtgggctggaaggacaggccagaggccagactgcatggggccagactggccctgattccagccttggacttcactctgtggtgataggagaccctcagagggacttggggaggggttgtgtgttttcctgggagccacagaaaagtgagtgacactgggctgaggaaggaagtcacaggacccaccagtgtctgcgcaggagtcccagtggaagagcagaaaaaatccaggagaagatacaaaagtattcaaaagtaattcaaaatagcaaaaatccaaaatattcaataatttaaatggtattacttaaaatagtattataaataataaaaatatattattaaaaataatagattactggctttaataccaatgtacagaaatccgttttatttctatagaccagcaacaaacggaaaatgtgattcttataaagatgccagatgtctgcttctattaagggcatacagggaactcggaccagttagcaaatctgaaagaagtattttaaaaatctgtttgaaggtaatggagagataccagagtggtgaataatagccaccaaagtagctgccatttgcagtcacttccccctgagcccctccccaaaagcatttgctgattctgggatagtcagtgagaagctgagcagaaatcctgacaatcttagagctagggagaaaaactcagaggccaggactcaccaggagaaaggcgcttgggaagtacatgcactttggattagaaacccatggactttccttataggagcaaaggtgaaggaaatcatccctcacaggaactgcaaccagatgcactttattcaggtccttagaaaagctcaacctctaccactgggttaagttgatctcagattttgaattccaccaggaaccagccagaagcatatgaaaaccgtctctggaaaattataacgtcattttaggttcagttgctcaacccaatacttctgaatacagtatccagcacacaatcacaaagaaccacacatacaagtagacgaagcaacatgagtaagaacagcaaaaacggaagatcaacagggactccagacctcagctatgggaattagcacatgaagactttcttaatattttttatttcaatagcttttgggatacaagtagtttttggttacacggatgaattctatagtggttaattctgagattttagggcacccatcatccaagtagtgtacactgtacccaatatgtagttttttatccctcacctcccccacccacttgtaatcccagtgctttgagatgccaggaggtttgggaccagcctgggcaacatggcaagaccctatctctacaaaaaatgtttaataattacctgggcatagtggtacttgtctgtagcccccactatctgagaggttaggtgggataatcacttgagccagaagtttggggctacagggagctgtgattgaatcactgcactccagcttgggtgacagagtgaggccccatctctgaaaaaaaataaaaataagtaaaattttaaaactgataaaataacagtgctactaggtttgaagagtttaaacacaatctgaaaatttcatcataaattgaaaactataaaacacacatagcagatcttttttaattcagaattttagaacagaagtatacaatgaagagctcaaatgatgggtttaagagcagacttaacacagctgaagaaagaattagtgaatcagaagttggatcagaagaaaatatctagaatgaaggatagacaaaaacacagcatggaactggagggttaaaagaagagagtatccaccgataaggtctaacatataggtagttagagtcccagaaggagaagtgagaaggaagagaagaaacatttttaaagaggcaattgcagaaatcttcccaaacttaaggaaatatcctaccccatcattcatatggaataaaatacgtagaaaatctcatctaggccaggcgtggtggctcatgcctgcaatctcagcaactttgggaggctgaggcaggcagatcacttgaggccaggagcttcagaccaacctggccaacatggtgaaaccctgtctctagtaaaaatacaaaaattagctgggtgtggtagtactcatctgtaattccagctgcttgggaagctgaggcaggagaatctcttgaacccgggaggtggaggttgcagtgagccaagatcgcaccactgcactccagcctgggtgacagagcaagactctgaagaaaataaaataaaataaaaccacatctatccataacattataaaactcctgaagactaaaaaaaaaaaagaggctatcttaagagtagccaaggtcggggaagctcacaatacctttaaaggaggaccagtgaggctggcagctaatgtcgtaacagaaacaagaacagaatgagatgctgtcttaggctggcagctaatgtcgtaacagaaacaagaacagaatgagatgctgtctttacagtgctaaaagaaatacctgctaagccagggaaactatctttggaaaataaagatgacttttctttttctcttcttttcaattttcattccctagggctatcctaacaaggaccgtaaagaacagaagtgtattcttgcacagctctggaagctggaagtctaaactcaaggtgtcaggagggctgggctctctctaaagcctctagggtaggatgcttcttgccccttctagcttctttttttttttttttttatttctaaaacgactttattgctaagaaccatgattattacttaaaatagatttttcagttactgatataaaatatgttccttttgagaacacatggacacatagaggggaatgacacacactggggcctattggagggtggaggccgggaggagggagagggtcaggaaaaataactaaggggcactaggcttaatacctgggtgatgaaatcatctgtacgacagatccccatggcacacgtttacctgcgtaacaaacctgcacgtgtacccctgaactcaaaataaaaggttttttaaaaaatgttctttgtgcttctaaaaatgtctaaataatcctatatgcatttttcttatttgcctcttatgcgtagcttacagatttaaaagtacaatgttgcttgttttgtattcatcttggtggatctaataatttacaaataggacataggcatgctgatacagtcagcagttaattcctccttctgacagtggggtttggacatacaaccagcaatgcgtgcaggtggagagacatagaggatctttttttttttttttttttttgagatggaattttgctcttgttgcctaggctggagtgcaatggttcgatctcggttcactgcaacctccgtctcctgggttcaagcgattctcctgcctcagcctcctgagtagctgggattacaggcatgcgccaccacgcccagctaattttgtatttttagtagagatggggtttcaccatgatggtcaggctggtctccaactcccaatctcaggtgatccacccgcctcggcctcccaaagtgctgggattacaggcgtgagccaccacgccctgtacgaagttttaggtcatctgtgattcacaaacgaccggcttcgagaagctctggaacgtgccaaacaaggcactgtggagtagtttggctccatgtgacctaagagaggaaaatctgttgtctttgggaacattacacaattcaggtaagatatgtgcatattctcaagggaaatttaaggtgctcatactcaataaataaacacaagagcagcgttacagataatattcaaagagacagaggcttcttcatacctgatgtggtgggaagaccactggatgactggacactgagatacgtggttgacagtctcgtctctgaaacacatgatttgagttttgagcttagacaaacttgtttttctccagcatcagttttttcatcagtaaacaataataataatactgtgtttatgccaacatcattaggaagatcactttaaatggtgtatataaaaggatccatcaactttaaagccagtacccaatatacactcatagatatcagcgtattacagaaaaatagtatttattttgtggtcaagcatatctggattctaatcccaattctactaattgctcccttatcttttgtgtcccgtgatcatcaatgtttttatcattagaatcttgataatatctagctcaccatgtttttctggaaattatatgagataacatatgcaaaggacttagtgcagcaccagatgtgactcttccagctcctgtggttgccgactgccctgggtgttcccgggcctgcagacgcatcgctccagcctctgcctctgtcttcatgcagtgttctccctgtatctgtgtctaaatgtttctcttcttataatggcactggccatagtggatctaggacccactctactgtagagtggcctcatgttatcgtgattatatctgccaagtccctctttccaaataaggccacattctcaagttcttggtggacataaatttgggggtgactctattcaacccagtatactttctcttcctgagaagagcagtttggactcaaagctgttcggtgggcagatcaagggttggggggtgtctgcatggtgggggtaggaggagagatgcaggggcccagagcgggaagccagtgtggagtccaggctcggagcacccacatgagcagaggtgggcagcctgatatagaaagtcagatcttaagtggggtgagaagggttccagatggggaggagtgcacactagaatggagggggagccagaatggcagaggggaagggagtactggcagagagggcaggttggtcacacaaggggattggtcaagtaagtaaatacaccgaaaagaatagaaaagagagaaggcagttccaaataccaaaagggagtgatatggtttggatctgtgtccccacccaaatctcatgtcgaattgggtttcaatctgtgtccccacccaaatctcatgtcgaattgggtttggatctgtgtccccacccaaatctcatgtcgaattgtaatccccggtgttggaggtggggcctggtgggaggtggttggatcacgggggtgggttctcatgaatggtttagcaccatccccctagtgctgttttccttatagagtcctcccgagatctggctgtttaaaagcacgtggcacctccctggtctccttttctcctgctccgggaatgtaagacatgcctgcttcccttcaccttcaccttccaccatgattttaagttcctgaggcctccccagaagccaagcagaagccactatgcttcccggacagcctgcagaaccgtgagtcaattaaacctcttttccttataaattacccagcctcaggtatttctttatagcagttcaagaacagactacagggagaaactggaatgaatcctatggtactggattggaatttgatccgtaagtttgaattcatggtttctaacataaatagccattatataggagttgtcctgatttgtagaaaacacactaaaatattcatgaaggaggaggtgtcaggtcaaaaaaatactctcttgaatgattcagataaaaattgtttgtacttttcttgcaacttccaatttttggctgaagctaaagaaatattttcagacaaaacagaatatttgttgccagaagacacacactaaaggaactgcttaaagataggtttaagaaagaaggaaaatgatcccagaggaagcctcagagatgaaaaaaagaatttaagagtgtaaagtacacaggtgagtacattttaagagttattaactaataacaatgatgatgtgatggatagtgaggatagaaaattaaaacaattcagatgcatagcaataacatataagtaatgaagggctaataacactgcatcatcatatctgaaaagaagatcaaagaattagtgacaaatttcttgataaatttaactaatagaaaaaaatgcaactgtaagaaagtccatccaaagggaagcaaggaaaaagggataaaagaagaccaacaaggacagcattatctcacttatttgtggaatcttaaaagagttgagctcataaaagtagatattagaatggtggttaccagaggctagaggcagagagggagagaatggagcgtggttggtcaaagggtacaaagtctcaggtagacaggaggaataagttctgagatctattgcatgacagagtgactatattcagtaataatgtatattttgagataactaagagagtaaattgaaagggtctctccacaaaaaaataagtaaatgaggtaacagatatattaattagcttgattcaatcattccacgttgtttacatgtatgaaaatattgtggctgggcacagtggctcacacctgtaatcccagcactttgggaggccaaggcgggtggatcacttgatgtcaggaatttgagaccagcctggccaacatggtgaaacctagtctccactaaaaatacaaaaatcagctgggcgtggtggcacatgcctgtaatcctagctacttgggaggctgaggcatgagaatcacttgaacccaggagacagaggttgtagtgagctgagatcatgccactgcactccagcttgggtgacagagccagactctgtctcgaagaaacaaacaaacaaacaaaaaacattgcattgaactccataaatgtatctaattatgatttgtcaattagaaattatattaattttaaaaccacattgctagataggagatttaggctcaaatatatcagtatttacattaaatgtaaatgggctgggagtaatgtcactgaaaatggcagctccaaaaatgtgtcccttcacgaaagcaacaattaagctggcaaaaaactgacagaattcattttttcataactctagaatataaccaaaaacttaaaacaaagggtgcttaatgaagaaagaaactgctaaatttgggtaagagagaattgtggaattttcttacctgcctataaagccctcattttccagctcagaagtagccagggcaacagcagcccatcttcccggtgtggtttgctggtgccagagggagtaaaaaagccgttgtcatcaaagaattgtgcttgcgaatctcaacctatctgacagctctctgagggatcagctcaggatcttgcctttgttttgctcaccctgctcctgcccttcctcccctgtgtcctacccccaacaaacacacacagaattctctacaggatggaacagcctcctgggcagcatacatggaaagtatttaaaggtatatacttgtcacaaccatctaggtcaacagataatagaaagggaaaacagtagacaggctaaaacgcctaggaagaaaaaggctaaggaaggaaacgtggggaaatgagaactttaaaaaactcccacatatactaaggaattcaaacagccacatgtatttctagatgtatgctcagataagacctgagaagaccctaagcttttacctctggctggtcattaggcttcacatgagcaggaagtaaaggctaaggcagagttgtaaatgacctggttaagtgttgcagcagtgctctaacccagagccagcctgcaaagaacaggagggtcttcttcctctttctttctttctttcttcttttggcttaagaaatttaagaagatctgtgaaaacactagctgaccactaaactaacataacaaagatttcagtggccacatatgacaaaagtcttcacaaaagtagtttagacaagtcactaaacaactataacacaaaacaagcagcaataacaaaccctagggagaagaaagaatctagtttcccatattataatatttattcaaattttccagtttttaacaaagaagtatgagacatgcagaaaaacaagaaatatgaccctttcacaggaaaaggtaaattaacggaaacttcccctgaggaagcccaggctgtgaacttactagaaaaaggccttaaatcaactgtcttaaatattctcaaagagctaaaggaaaccaggagaacaatgcctcagaactagtgattatcagtaaagagatagaaattataaaatagcgccaaatagaattcttgaaatgcaaagtactgtaactaaagtgaaaaactcactacaggttcaagaggaaacttgagcaggtagaagaatcagtgaacttgaaggtaggccaattaaaattctcccaactaaagagcagaaaaaagataaggaaaatgagcttaatttgaacaatatgatcaataaatttgatctactaggcatatacagaccttagaaagagcagctacagaatgcaccttcattttgagccaggtggaatattcacacattataccatttatgaccacaaaggaagatttaacacattttgaaaggctaaaagtatttagttatgatgtgatcacagtgctaagtcagaaataaataacaaagaggtaagttaaaatacacatatgcttggaaaataagataaaaactgctaaataaccctgaagctaaagaagaaatcataatggaaacattaaaattttaaaaagtttaaaatatttttaactgaataataagggaaatactacatatcaaaacatggtgagatgcagcctaaacagtgcttagaggaaaacttatagccttagaagcatgtattatgaaagataaaatcctgaaagttgatgaactaaaaattcacccgaagaagttaaaaaagaatgtaaaaccaactgaaaatagaaggaaagagatcattaaaataatataagaaaatgattgaactgaggataataaagcaaagaaatgatcaaccaagtgaattaacaaaattgataaatctctgataacacttgttggggaagaaaagagaaaatgcaaataactaatgtcagaaataaaaagggacgacacacacatcctgcagatgtttaaaatgtaaaaaagaatatttgggaaagctttaagctactaagtgtgaacaattggatgaaatctaactggataaatgtaagtatttaaaaagtttaatctacaatttataaccttaccccagagaaaactgtgaaccagacatgatttcataggggatttctgatcaatatttagggaaatatcaacactcttagacaaactcttccagggatctgaagaagagtccatgccctgtaagctactgtatgaagccagtgtaatctgagttccgttgggactgcaaccgataaagtaattcatcacagaaatgacctcaggaaactggccttcagaacgggcttctgggaagttgtctcacatatttgaggaccacaggcataattcattgcccgcaggaaatggggcagccatacatgacatgtggtggcaccatgatgtctcagactgtcactgacaggagtgcatccaaagtacaaggagagaaaaaggcatgggcaggtcaagatgcagtagcactttgttgtcatggcaacaagagggctgctaaagattatggagtcaccaactttttctcatgaccatagagagtgtacacaggggaaaagaaacagtgaaagctatgaacatagcaaaaagaacatatacagagagccatggacgcttgaaggagtcccttacctcctggtcatgggtcagatagagctgatacccaaccaattcactacagatgaaagtcttagaaattgtggggctacaacatgcaaaggtttatcatcatcccacataacactaaacaggaggctctaactgcattcttgttggacatgtatccaatttcagaatagtgttccaagaatctgcctcctaggaccacttttgttggtgtcagggatccccagaagctgatcttgagagaatattttaagtggacagtttgtttgagaggtgatgccaggaaacactggtggaagagttggggatgaaaggaagccaataaaaagtgcactaataagcatgttaccatggaaataactgtaactgaaattcaccagggccctcaggaagccagtgtcaaacaaacaccttctcagtacgtccccccaacacacaccagcagcaagggagtttggggtatttatatgctaacttattagtcattggttgatggcagtttgtgcagagcaaaatcctgtgcaacttcaggcctgttgtgcacagaaaccctgactaccagagaaaaccctcaggcaagaagatgcagatgctagcagtttgaggttacaccagtatacactaaagtgtaaaggcccaaggcacctggagtgcaccaaaaactattgccacaggtgtacaaatgttcaaattaacaagaaaatgccatgtcatcctccaaagtcatggcaccaattcacattcccatcagcaaatctgagaccttatccaataccaatatcctgacctgaaactagatcatcatttatttgcatctgtgtttccttcttttatgtatccttttgtctcttgatcagagttagccacactctttattttgtatgcatcattccctccctttccataaaaaaaatttatcaaatatggccgggcgcagtggctcccatctgtaatcccagcacgttgggaggccgaggcaggcagatcaccagaggtcaggagttcaagaccagcctggccaacatggagaaaccccgtctctactaaaaatacaaaaattagctgggtgtcatggcacacacctgtagtcccagctactcaggaggctgatgcaggacaatctcttgaaccttggatgtggaggttgcagtaagccaagatggcaccactgcactccagcctggatgacagaggcagactgtctcaaaaaaaaaaaattatcaaatatgtatgtatatctgaacttgtctgattctgtttgcccttgagctatgtaaaattatatcatactatatgcagttgtctgcattggcattttttaccaatttttttacatcttgtttccaagattcatctatgttgttgtggggagttgtaatttattcacttttactgctgtataggtttccagaatttattttccttggataattgtttcaaaatttttctattattagctgccttactattgcattcttgttcctgtctgttgagatcctgttataggctgaatatgtcctcacctctccccaaattcatatatggaagccctaatcctcaatgtgatggatttggagatggggcctttgggaagtgattagtttttgatgaagtcaggagggtgggcgcccacggtggtggtggtcatgagggtggtcctcataggaagaggaagaaacggaagagcctcactctctctgccatgtgaatgcaccatgagaaggtggccatcttcaagccaggaagacagccctcatcagaacccagccacgctggcacccagattctggacttccaacctccagaacaaagaaaactaaatttttgttgtttaagccacccagtctatggtatttttgtcaggacagcccacactaagatggatactagtgggagagtttctctatgaaatatgcacaggagtggaagtactgaggtatagaatattatccctatttagttttctggaaagtgttttatcaagcatgttaccatgacaattaactagagtctaacacctctggggaattttgattatcgttgtagaatatatgccttagaattatcaccagggacaagtgacctcaggcatttacacacacaagctcccatcagccatgggttaagagctgctccaatgagtactaattcctggcacttctgttctgtattcatttaggctctggaagccacaggaagcaatcagacaaaaagtttcaggtgtcgtggctgggcatagtggcttatgcttgtaataccagcactttgggaggctgaagcaggaggatcacttcagcccaggagttcagaaccagcctgggcaatatagtgagaccacatttctacaaaaaatttaaaaattagctgagtgtggtgatgcatgcctatagtccctgttactcagtaggctaaggcagaaggatcgcctgagcccaggaagttgaggttgcagtgagccgtggtctcaccactgtgtgcattccagcctaggtgacagagcaagactctctctctctctctctctctctctctctctctctatatatatatatatatatatatatatatatatatatatatatatatatatggctgggtgcagtggctcatgcctaataatcccagcactttgggaggccaaggcgggcagatccctgaggtcaggagttcaagaccagcctggccaacgtggtgaaaccccacctctactaaaaatacaaaaattagccaagcatggtggcgcatgcctgtagtcccagctatttgggaggctggagtaggggaatcattgaacccagaaggcggaggttgcagtgagccaaggtcatgccaccacactccagcttgggcaacagagcgagactctgtaaaaaaaaaaaaaaaaaagaaaagaaaagaaaagaaaaaagaaaaaaaaaggaacacagaaggcacaaacagcatccactacagtcacccctcgcatgtctaaggtgcacttgcaccccacagtgatgtcaccacttcctattatttcctctgaaaatgaacccattttgagatgggcttgggccagaattacccttacccactgatccctgtaaacactcactctaacaggtgaggagtgaagagaatagggaggcttctatggtattctggtccctcaattcattatctgggttcgtggaacaactacagattctgctgttgcaactgatcccgaggccttcattggcctttgtcatctcccttttggagataggtttctaagggtttcccttgaccatggcaagaacttctgctgggctatgctctatgcctggtgggatgatccaaacctccattttctgcagaattacagaccccaacaaaaatgtccttattccaggtcttgaggtcccactccttcattattggggtgctttccttagtggaagagaactttgagctcctggattcagccttctttgtaatcccactactcttacaatcaggttctgggcccaattttcagtagtttcttctccagctgcaggagatggagttttttctaatgctgtgatgaaggttttctgaaatttgcaccatttcctgagttgaaaggggttcaactgagcctgttgttatatttcttcagtgattcaatttcactaaacaacaaccaaactattccaccattcttatgatcctgtctttctcaaaggccagagaaattacataaggagcacatcccccaacactgtaacactttctatctatgtcccattatcataatgccaggagaagttatttacaaagggttatttacaaaagtgcaagtgagatgtaagagaaccactagggatagtgcagtaagccaggttaggtaacaggagggctgttgctgcccacaaaaccaaatctcaaggtgttttagacaagacacaaactgtaaaagacttattggtaaatttaactatacaaaaattaaaaagtcatgtccatcaaaagataaagaaagtgaaagacaagtcacaacccgggaaaatacatatgcaacacctaaacttaacaaaggaatattatcaaaaatgtgtaaactcctacaaataaagaagaaaaggataaatatcccaatagaaaataaaggccaacagcttaggcaacatggcaaaaccccatctctacaaaaaatacaaaaattagctgggcatggtggcacacacctatagtcctagctacttgggaagctgaggtgggaggatcgcttgaccccaggaggtcgaggctgcagtcagccatgattgtgccactgcactccagcctgggagacagagtcagaccctgtctcaatcaatcaaccaatcaatcaagtaaaataaagaccaaaagacacgaacaggtatttcagagaataaaaaaaatacataaggccaataaacagtttagaaggtgtcagcttcactagcaatcagaaaaaaagtcaaatcaagagaagaatattcaagaaatcatgttagcaaaatgatgttggcaaaaattaagaagttgaaccataatcattaagacatagattcacagcctccaagtacgaatgatcctccgacctcagccccccaagtaactgggactacaggtgcatgccaacatgcccagctaattttttttcttttttgtttttttcatttatttgtagagactgggttttgccatgttgcccaggctagtctcgaactcctgtgctcaagcaatccgccccatcttggcctcccaaagtgctgggattacaggcgtaagccaccccaccgggcccagaatgctttcttaatctaaaaactcccctaagtggccaggcgcagtggctcacgcctgtaatcccagcactttgggaggccaaggcgggtggatcacgaggtcaagagatggagaccatcctggcaaacatggcgaaaccccgtctctactaaaaatacaaaaattagctgggcatggtggcgcgtgcctgtattcccagctactcaggagactgaggcaggagaactgcttgaacccaggaagcagaggttgcagtgagccgagatcacaccactgcactccagcctgggcgacagagcgagactccgtctcaaaacaaaaacaaaaaccaaactcccctaagtagggctattttcctgtccctgatgccaccttgacaacataagtcaaccactgttccacatttgtcttttaggaacgccccaggactgttggctgctgtccatggtgctgccttggtcagacacgtggtgtccaagctgctgtccgtggttttgctttcatttttctgtgtagcataatgatttttttgtttatttctttgtttaattaattgattttctctttttagaacagcgctaggtttgcaaacaaattgaacaaaaagtccatagactttttacatacccagcgcctctctatacatatacagtgtcacctactgtcattgtgcatctgtatggcacatttgttacaaatgatgagtcgatattgatacattatcattaactgaaatccgtagtttaccttagggttcactacttgtgttgtatagttcatgagttttgacaaatgtgcaatgtcacgtatccaccatggcagtgttatgcagaatagtatcccctatatttcactattcattcctgacccctcaccccaaaccctggcaactgctgatctttctactctctctgtagttttgcctttccaaatatatagtttggaaacgtacattatttagccttctccaactggctgctttcacttagcaatatgtatttaagattcctccatgcgttctgtggtttaatagttttttgtttttgttttttgtttttgatagggtcttcctctgtcacccaggctggagtgcagtggtgcaatcatatttcactatagcttcagcctcccaggctcaagcgatcctcttgccttagcctccaacacagctgggattacaggcaccaccatcatgcccattttttaatttttcatagagatggtatctggctatattgcccaggctgatctagaactcttgggctcaagtgatcctcctgtctcaacctcccaaagtgctgggatcacagtgtgagccaccatgcctgcccttgatagctcactttttattgctgaataatattccactgtccggatgtaccacggttaactgaaccattcacctgtgaatggtatcttagttacttccaagtttggcaattgcgaggaaagctgctataaaagctcacgtgcagatttttgtgtggacataagttttcaactcatttgaataaatacctaggagtgtgattgtatggtaagataagatttagcttttaaaactgtcaaactgtcttccaaagtggctgcaccatttgtattcccaccagcaatgaaagagagttctttatgcctcacatcctccacagcatttggcgttgagagctttttgtttcatttgttttctttgggattttctccattctagtagctggtcgtggtagctcattgttgttttcatttgccatcccaatggcacaccacgtggagcatattttcatgtgcttatttgccatccgcaagccttctttgctgaggtgtctgttcagatcttttgcccatgttttaattgggttgcttgttttcttattgtttaaatttaataatggttctttaagtgttttagatacaagtcttttatcagatatatattttctcctcccagtctgtggcctgcctttttcattctcttgacaatgtctttcacatgtagacattttttcattttaataaacctcagttaactaatttttctttttatacaaattgtgatttttgtgttatatctaaaaactcattgccaagcccaagatcatctggattttctcctgttatcttctaaaatttcatagttttaggctttacatttgggcctatgatccactttgagttgatttttgtgaaaagcataaggcctgcgtctagattcttttttttttttttttttttttttttgctttggtgccatgtagtgaaaagatgacgctttctccattgaactgcctttgctcctttgttgaagatcagttgactatgtttatgggcgtctattatcagaaaattttgttctatggatttatttgtctatcctttcaccaattccacactcctgattattgtggcttcacagtaagtcttgcataaaagttgggaagagtcagtcctctaactttgttcttcttcaatattgtgttgtctcttctgagtactttcccttcccatataaactgtagaatcagtttgccaacatccataaaataatgtggtgggaatttgattaggattgtgttaaatctatatataaagttgggaagaactgacatcttaagaatattgtttttcttttcatgaacgtggaatatctctcgactgatttagatcttctttcatttcttttatcacagttttatagttttcctagtatatatcttgtatatgttttattagatttatacccaagtatttcagtttctggtgctaatttaaatggtattcggttttccactttaaatcccaattgttcattgctggtatatagggaaaaaatggcttttgcccattaaacttaacgtccttcaaatttgcttttaggccattcttgcgttgctataaagaaatacttgaggttgggtaattttttttttttttaaaagaggtttaattggctcacggttctacaggctgtacagaaagcatggcagcctctgcttctggggaggcctcaggaagcttccaatcatggcaaacagcaaagggagagaagccatatcacatggccgaagcagaagcaagaaagagactctgtggagggggaggtggcacacacttctaacgactagatctcgtctgaactctgagcgagagctcactcatcaccaaagggctggcacaaaccattcatgaggatctgcccccatgatttaaacacctcccagcaggccccacctccagcattggggattacatttcaacatgagatttgggtaggaacaaatacccaaactatatcattgctataatcacttattagttccaggaggttttttgtcgattatttgggattttctgcagagatatcatgtcatctgatgacaaacgcagcttcatttcttctcctccaatctgtatatcttatacttgcttttcatgttttaatgcattagctaagacttccagcatggtgttaattgggactggtaaggggacctactcacctgttgcttctcaccatgaagtacaatgttagcagtaggttttttgtagatgtcctttataaatttgagagtattcccctttattcaaagtttgctgagagtttttatgataaattgagttgtattttgtcagacacttttttgttatctattgatatgaccacatgagttttcttaatcttttgaagagatggattatattaattgacttttaggtggaaagcttatattaattgattttgaatgtccaatcagccttgtttctattactgagagttctccagattaacagaaccaatagaatagggtgtgtgtgtgtgtgtgtgtgtgtgtgtgtgagagagagagagagagagagagacagagagagagagagaattttaaggaattggctcacatgggtgtggaggcctgcaggtccaaaatcatcaaatcggtagtgtggaacctgacctagggaagagatgatgttccagcttgagtccaaagatcttctggaggcaaaattccatcttcctcaggggacctcaatctgtcttttttaatagtcttcaactgattggatgaggcccacccacattttggaggatgatctgctttacttaaagtctaacaacttaaatgttaatcctgtctaaaaaaataccttcacaacatccagactgatgtttgaccaaatattgggttccaagacctagccaagttgacaagtaaaatcagccatcatatttgcatacctgggataaatctcaatgggtatattattttttatacatggttggatttcatttgctagtatttcattaacattagattttacactgtcgatattgaaggtaagcaatctttgaaaaagactactagaactcacccatagctgtttttctccttcctttgactcctgctagactgtgtttcccagtgccatcacatgtggatggagacctgttaccagttctgttcaatgagtaatgtgaataagtcagacagtactttgaagagttgtgatgctgtaacaaaaaataatattatgtggctcatgcctgtaatcccagcacttcgggaagctgaggcaagaggatgatttgaactgagtaattcaagatcagcctgggcaacatagcaatactccatctctagaaaaaaaattaaaaattagccaagcatggaggcacatgcctgtagtcccagccaccagggaggctgaggtggaaggatcacctgagcctgcagaagcagagggtgcagtgagctgagatcataccactgcaccccagcataggtgacaaagtgagactctatctcaaaaaaataaaataaaaaacttgccttagaatatgagaagcagttagcaaggaagctggtgtcaggctggaggactgcagatccttgtcatgccttaataaatcagttgttaacatttggaaggcagagtccctgcctattcagcctgcagctctagggtaagagattgcaaaatcaagtattaagagtttgttggctactattagctcccttcattaattaactagagaaaagaggttaggtcagagaattggtggctttgtaagcaaaaattttaaaagaatagagaaaatccaaaacttacaaggagggagtggaaaagctggcttcttctagaacccaaacaagacaggacaccaagaaaggttttagatgacgaggatggccaaggtgtgatttgcagtgtggccttcccatccatggcctctttcacagataatctaatttttaatttattatttttgtggatacataataattatatattaatgtatttatggggtacacatgatattttgatacatgcctagaatgtgtaatgatcaaatcggggtagttaaaatatctatcacctcaaaatttatcatttctttgtgttggaaacatttcaaatcttctcttctagctattttgaaatatacaataaattattgttaactatagtcaccacactgtgctgttaaacactagaacttattccttctaaccaactgttttgtgtgtgtgtgcgtgtgtgtttttttttgagacagagtcttgctctatcatccaggctgaagtgcaatggtgcaatctcgactcactgcagcctccgtctcccgggttcaagcgattctcctgcctcagcctcccaagtaggtgggattacaggcacatgccatatgccctgctattttttgtacttttactagaaacagggtttcaccatgttgtccagattggtctcgaactcctgacctcaggtgatccacccacttcagcctcaaagtgctgggattacaggcgtgagccacaacacccaacctacccaattgtatttttgtatccattataccaacctctcttatctcctccccacagcagtgatccttcccagcctctggtaaccaccattctcctctctacctctatgagatccacatttttagctcccacatatgaaagaggacacatgatatttgactttctgtgcctggcttatttctctttaacataatgacctccctttccatccatgttgctggaaattataggatttcattcttttttatggctgaatagtattccattatgtatatataccacattttctttatccatttatccattgatggacacttaggttgataccctatcttagctattgtgaacagtgctgcaataaatatgggagtgtagatatatctccaaaatattgatttcctttcttttggatatatatgcatcagtgaaattgctgaattatatggtagttctatttttaattttttgaagaacttctacactgttttcaataatggctgtactaatttacattccaactagcagtgaatgagcattctcctttctccacatcctcatcagcatctgttcctttttgtctttggtaataactattctaaatggggtaagataatatctcattgtgatttcgatttgcatttcctagatcattagtaatgttgagcattttttcatgtacatgttggtcacacttgcatgtcttcttttgagatatgcatattcatgtcttttgtccattttaatgggatttttcttgttgtggagttgaattccttgtatattctgaatattagtcccttgttggatgaattttatcccatcaaaaagttgactcttcactctgttgactgtttccttgctgtgcagaagctttttagtttaatatagtcccatttgtctatttctgtttctgttgccagtcattttgaggtgttagccataaaatatttgcttatgacaatgtcctgtagtgttttccctgttttcttctagtagttttataatttttagtcttatgtttaagtctttaatacattttagttgtttttttaattcagtgagagattgggacctagttttattcttctgcatatgaatatccagttttcctagcatcatttattgaagaggctgtcctttcccagcactttcattgaaaatcagtaggctataaatatgtggttgttttgtttctaggttctcaattctgttccattggtctgtgtctatttttataccaatgtcatgctgttttgtttcctatatcctcatgatatattttgaagtcaggtagtatgatgcttccagctttattctttttcctaagggttgctttggctatacaagctcttttttggtttcataataattttgggattgtttttccatttctttgaaaaaatgacattgatattttaatagcgattacattgaatctgtagattgctttggatagtatggtcattttaacaatattaattctcccaattcatgagcatggaaggtctttccatttgtttgtgtcctcttcaatttctttcatcagtgccttgtagttttccttgcagaagtctttcacctccttggtgaaatttatttatatggatttttgtagctattgtacatgggattgccttcttggtttctttttcagctagttcattatcagggtatagaaatactactgactttcatatattgatttgtatcttgcaactttactaaatttatttatcagacctaagagtttttggtggagtctttgggttttttcctttcagccagtatatatcttttaagtggaaaattctatctgtttacattcaaggttattattgatacgtggggacttatttctgccattttgttggttgttttcttgttgttttgtctatcctgtgttcctttctttctctcttattgtttatcattgtggcttggtggttcctgtagtggtaacatttgtgtcttttctcttcctctttttttgtatttgccgtgcggctgtaagttttatacatttatgtgttttcacgatggtagagatcatccttttgcttccaagtatagaactcccttaagcatttcttgtaaggccaatatagtggtgatgaatttccccaggctccagtggctcacataggtgctggctgtggtgggcaggatgggatgatcctttgccccacccagtaacagtagcagtgtgacgggagagcctgtcctcagggcatgtgaagtgcacagtggccctgctatggggtcactgcccgtggcatgtacttcagccctggaagcagcagcagcttcatcatagcttatttaattcagcggaccaagagcataaacacctgagcttcctctttttatctgctcattctgcttcccaagttctcaaggacttatgtggacagtcttcccgcacttgccaaaaactcagttcattctgttataaatctcagtgcctctacctgctacagacacggagctcaccccgtcagtggcaccccttctgacccagaagtgtccctaggtgtcttcgtggcctctgccatgggtgctggtatgccattgtgggaggtggggggcagtgggagtcaggaggttgctcctctgggtggagctgcctgccaatcaagatcctgactctacctccaccccatgtgaccaggctgcaggggcaggccttagtgggcaccggtggtgccctggtcacaagacaccctggaggtgaagggggccttccaattctaaagctgtagctgcagacttgggcagtgcaaatcctgtcaggaccaaacaccaaattctatcacccactcaagcttgaagttaagtgggagggagagagtccctgcaggacccttgctatagttggatgtttgtcccccaagacttcatgttgaaattcgacccccagtgttggaggtggagcctggtagaaggtgtttggattttgggtgcagatcccaaaatgctctcctttgagggtgagttctcactcttattcctgctagagctggctggtgaaaggagcctggcatctcccctctttcttgcttcctctctccccatgtgatctctgcacataccatggttcttgtacagcctgcagaaccatgagccaaataaacctcttttctttataaattacccagcttcaggaattcccttatagcaatttaatggacaaagacaacacctgacagccactctcccacatgcctccagcaatgctctgtgggtttgcacctccaactaagtcatttaggcaggatttaatggaaggacaccttgctgacacagatctgggctcagtgcaagagcccctgggacactgaggactccagagctggctccggcagagtgaagagtagcagaatatgccactttcaaacaagaataattttgagccgaagacaattaaaaagaagcatacccaagaaaaaaatccctgcccactccctttctaccaggaaaagcagaggattcttagtccttggagacaactctagatgcttatcagcccagagaaggcactagaggaatctacacaacaaactagcaagctatcttctgttagcttcccctatatatttacttcccagaatttgctgccctacaggttgaaagcccctttcctttgtcttgccaattctgtaaaaatgattgcccatttgctaagatgctccgtaagctcacgttccaaccgcccctttgaattcctcatctctgacggctcccacatagaggcataatgcacatgttagtaaacttctgtttgtttttctcatgttagtatcttgttaatctaattgacaagaccccagctaatgaacctaagatgggtagaagggggaaaaatgtttcttcccctacaagagcaagcacctccccacacactggaggaggagagagagccgctccgagatctgcaggaacagaggctccttgggctgtctgttgaggaaggcaataaggtccaggcaaccctgcaaggagagagccagggtcaccacttagcatcagcatccccttcctctgatctcctgcaggcagccagcccacccaaagagctgacccatccagcacccaaggagtgaggcaggccagagtccactccaagggcagaggctgtggggcaggtggggagccagcctggaggatgttagcagagccaggtgggtcatggcacccacagcctcggggatccagcttgagagagaccctgagggccaggcttgtcagagctgataacagatgagttgtgatgacagggctcattgtcagacactgtaggctgtcaggctgtcaacctgagcagactaccaagtcttcccatccgcaaaacagctcaccccagctatcaggggtgggcacccagaggagagaccattctcatctccacctgaactcttttggtgctcccagaacattctgatgctgaaaaggagtgtagaatcagatagaaacaattcctcttgtagcttccatgatggttggatggtaaagtacaactggctttgttagttcattttgcattgctgtagaggaatacctgaggctcagtaacttataaagaaaagaggtttatttggctcagggttttgcaggccataaaagaaccctggtgccagcatctgcttctggtgagggcatcaaggagcttacaaccatggcagaagttgaagggggagagagcatgtaacatggtgagagagagagcaagagagggaaagaggaggtgccaagctcttttaaacaaccagctcttgtgtgaaccagtagagcaaaactcactcatcaccaagaggatggcagcaaggccattcatagggatccatggcatgacccaaacatttccactaggccccacctccaacattggggatcacatttcaacaggagatttggaggggacaaatatccaaaccatgtcactccacactgtccccccaaatctcatgtccctctcacattgcaaaatataatcatcccttcccaatagtgcccaaaaaatctcaacttgttctaggatctactcaatcaaaactccgaagtttcatctgagactcaaggcatgccccttccacctatgaggctgtaagatccaaaacaagttgctacttccaagatacagtgatggtacaggcattgggtaaacatttcccttccttccaaaaggaataaattggccaaaagaaggagggggaagaagcaacaggccccacacaattctgagagaaagacattaaactttaaagctccaaaataatccttgactccatgtcctgcatccagggcacactggtgtgaggagcgggttcccaaggccttgggctggtctgcccccatggctttgcagagtgcagcccacatggctgctctcatgggttgtagttgagtgcctgaatcttttccaggctctgggcgcaagctgctggtggctctaccattcttgggtatggagggcagcagccccattcccagagctccactaggcagtaccccactggggactctgtgaaggggctccaaccctacattttccctccacactgccctactggaggctctgtgggagaactccacccctgtggcaggtttctgtctgggctcccaggctttcttttttgtctctctctttttttatttttatttcaatagttttaggtttttggttacatagataagttctttcatggtaatttctgagatcttggcacacttgtcacccaagcagtgtacgctgtacccaatatgtaatcttttatccctcacctccatcccccacttgccctcaagtatccaaagtccattatatcattcttatgcctttgcatccttatagtttagctcccacttataagtgagaacatatgatgtttgtttttccattcctgagttatttcacttagaataatggcctccaactccatccagtttgctgcaaatgccactattttattcctttttatggctgagtagtatcccatggtgtatatataccacattttttatccactcattggtcaatgggcatttaggctggctccatatttttgcaattacaaggagttgcaattgcactgctctaaatatgtgtgtgcaagtgtctttttcatataatgacttatttttttctggatatcgaatagtgggactgctggatcaaatggtgattctacttttagttatttaaggaatctccatactgttttccaaagtggttgtactagtttacattcccaaccaggagtgtaaaactgttcccttttcaccatatccatgacaacatctgttagtttttaatttttaattatggtcattcttacagaagtgaggtggtatctcattgcagttttaatttgcatttccccaaaaattagtgatattgagcatttctttatatgtttgttggccatttgtatatcttcttttgagaattgtctattcatgtcgttcgcccactttttgatggtattttttttattgatgatttgtttgaattccttgtagattctggatattagtcctttatcagatgcatagtttgcaaatattttctcttattctgtgggctgtctgtttactctctgattatttcttttgctgtgcagaagttttttttgtctaattaggtcacatttatttatttatttatttatttttggttttgttgcacttgcttttgggttcttgatcatgaactctttgcctaagccaatgtctagaagagttttaccaatgttatcttctagaattttcatggtttcaggtcttagatttaagcctttcatccatcttgagttgatttttatataaagtgagagatgaggatccagcttcattcttctacatgtggcttgccaattatcccagcaccatttgttgaataggacatcttttccccactttacatttttgtttgctttgtcaaagatcagttggatataagtatttgacttcatttctggattctctgttctgttccattggtctaatgcctatttttatacaagtaccgtgctattttggtaactatagccttgtagtatagtatgaatttgggtaatgtaatgcctccagatttgttcaccttgcttagtcttgctttggctatgtggggtcttttttggttccaaatgaattttagaattgttttttatatttctgtgaagaatgatggtggcactttgatgggaattgcattaaatctgtagattgtttttggcagtatggtcattttcacaatattgattctacccatccaagagcatgggatgtgtctccatttgtttgcgtcattgatgattgctttcagcagtgttttgtagtttttcttgtacagatcttttacctccttggttaggtatgttcctaagtattttattttttgcaggtgttgtaaaagggattgagttcttgatttgtttctcagcttggtcattgttggtgtatagcagtgctgctaatttatgtacattgattttgtatcctgaaattttactgaattaatttaacagatctagaagctttttggatgagctttagggttttctaggtatgtgatcatatcatcagtgaacagcaacagtctgacttcctttttactgatttggatgggcacccagactttcctatacaccctctgaaatgtaggtaggagctgccatgcctccttcactcttgtattctgcccacctgtgggcttagcaccatgtggaagctaccaaggcttccagcttgcaccttctggagtggcagcctgagctgcacctgggactctttgagccttggctgaacctggagcagcaacctcccaagataccacagggaagcaaggccccagccctgacccccataacaatttcatcctcctaggcctctgggcctgtgatgggagagactgtcccaaagacttctgaaatgcctttaagacctttttctcattgtctttgctattagcatttagttgccttttagttatgctaatctctctagcaagtggttgctccataaggcatttgggttccttgcctgaaaaggctcttttcttctcaacctcatggccagcctgcaaattttccaaatgtttatgttctgcttaccttttaattataaattctaattttaagtcattttttttgctcccatatttgatttaggccattcaaagcagccaggttacttcttggatgctctgctgctttgaaatttcttccaccagataccttaggtcatcactgtcaatctcagcctttcagaaagccctggaacatggacacaatacagccaagctccttgctgggggcataacaagcatcacctttactccagttcctaataaattcctcatttctacctgagacctcatcggcctggcctttactgtccatatttctatcagcactttgatcacaactatgtaaccagtctctaaaaagttccaaatttcccctcatcttcctgtcttcttcctgggccctccaaactcttccaacctctgcccattacccagttccaaagccacttccacattttcaggtatctttatagcaacaccgcactccttgctaccaattttctgttttcatccattttgcattgctataaaagaatacctgaggctggataatttgtaaagaaaaggaagtttatttggctcactacaagctgtacaagaagcataatgccagtatctgcttctggtgaggcctctggaagtttacaatcaaggcagaaggcaaaaaaggagccagcataggacaggcatggtggctcatgcctgtaatcccagcactttgggaggccaaggcaggtggatcacttgaggtcaggagttcgagaccagcctgaccaacatggtgaaaccccatctctactaaaaatacaaaattatccgggtatggtggtgcacacctgtagtcccagctactagggaggctgaggcaggagaattgcttgaacccaggagacagaggttgcagtgagtgagccgagatcacaccattgcactccagcctgggcaacaagagtgaaactccatctcaaaaaaaaaaaaaaaaaaggagccagcatgtcacatggtgagaatggtgagagggagaaagagagataagaaggaagtgtcaggtttttttaaacaaccagctcttgtgtgaactaatagagtgagaaatcacccatcaccaaggggatggcaccaaaccattcatgagagatctggccttatgaccccaatacctcccaccaggcctcacctccaacactggggatcatatttcaatgtgaaatttggaggggacaaacatctaaatcatatcaggaactccatggtactgctaacccccaaatctctaggggtgatattgagtgacatgcatggtactgaacgatacttggtcaggttctcccagtgaagcccaagcaccctcagggtacagggctgggaggcccaatcacaccagctcagcccaggacttggctgagtctgccctggtattagaaggtagcagacctagaactgggttgaacaagatcaggccactttgtgcatgttgccaaaggagaaactgaaggatcagaagtgggcagtaagttttcagtgagcccaggggtcataagtagcaaaatgtggaattcagtaaggggtagaggctggaagagttttgaggtgtgtgttagaaaaagcctaggtttccttgaagactatgttagaaatttggacattaaaggtgatcctggtgggggctcagaaagaaagaggagagctatagagaaaactcctgtcatcttggagaatacataaattgtcacgaacagaatgttgctagaaacgtgaatgttaaagatgcctctgctgaggctccagacagaaataaggaaggtgttattgaaaacgaaggtgacccttgtcatacagtggcaaagcacttggctgaactgtgttccgttttgtggaaagtagaaattatacatgatgaacttggatgtttagctgacgaagttcctaagaaaattgttgaaggcattcttgatttctccttgctgcttacagtaaaatatgagaagagagagatcagctgaagagggaagtgctgaggtggctgggtcagctgacccaagggcccttcaagctgatttaggctgctcaccttggtctcagaatcaccacggccatggccggtcagtcacaggtgatccagcagctgctgcaggccgagaagcaggatgccaagaaggtgtctggggcccacaagcaaaagaaccagaggctgaagcagcccaaaggagcagcttaggctgaaattgaacagcactgcctgcagaaggagaaagagttcaaggccaaggaagctgcagcactaggatctcgcagacccaggataaccatcctccagacctacttccagcagaacagggaagaagtcttagataaccttttggcctttgtctgcagcatccagccagaaatccctgaaaactatcgcataagtagatattaaaagagagaagcacctgttaaatggactggcactttagatgccctcatggaataagaagctctagttatattcttataagaagacattaacttatctctgtatattatagagtaggcccattcacttttcggagagaagcaaatccagtttctttgtacagacttagaatttatctaggctgggcatggtggttcacgcctgtaatcccagcattttcgggggccaaggcgggcagatggcttgagctcaggagtttgagacctgcctgaacaacacagtgagactgtgtctctacaaggaaatataaaaattagccagatatggtggtacatgcttatagtcctacctacttggaagagtgaggcaggaggatcacttgggcccggaaggcagaggttatagtgagccaagattgtgccactgcactccagcctgggtgctagagtgagaccctgtctcaaaaaaaaaaaacttaaagatttcatctttttacctcatgtttcttaggaatataatgggtaaatattgtctattttattatgccttttggctcaagcaatatatatacatatatatatatcactgttgatgttttctttcttgtatcaagtttaaaagaaaaagcaaaacaatcctttgaaaaaaggaaggaattaaatcattttttccttaatgcttttttgaaggtcaggggctttatctatgaaaaagtagtagtttctgtaacctgtgtgaagcatcagccagccttaaagtagtccattgctgctaataattagaacagtgaatattactagtataattgtttcagcttcttaatcaaaataactagatgatagaattcaagaacttgttacatgttattactcggtgtactactaatcatttaaaagtaaagcctatcatgcaaataaataaataaataaataaataaatacatgagaccagataaaatgaaggaagaatgactttgagatcagagccacaggtgaggaggctgttggggctgctgttgccaccttgggcccagagtgtggggccaccccagtgggcctggaagatggagcattaaccgaggaggatgaatcttgagacttaaatctaataaagggccaactagaagcccctagtgctcatcatcctcataaagaaagccagaaaagtcaatagacaactatattttaacgaaaataactgagggagagcactgagtgcatcagaggagtaacagaaaccctgataagcacagaaatgtgggctggccacatagaaaacagaaggaaacacacggcctccaccactccatcccccaaccaggattatctgggaatcaggaggaatttctgacatgaggaggtaagcaagaggatcccagtggcacccactaacacctcggacacctgcagacctcgccactggggtcccctgcagtttgcactggagggagttgcctggaggccacatagctgtgctctccaaaaaaggtgctgacaccgtaccccactcccacatggctagtgcttcaccacactggaataggaactatggctggagtgtgtcttacccaaggagtgagtagtcacagctccccttcatctctgaggctaagctgccactgaacaacccagactagtggtcccacgtccccaagtcaagctatgggcatctattaaacccttccccacagagccaagtggatcagctctacccactcctccccctgaccctttgagtccaagctaaagcagtttcttgcctcctagcaaaacaggactttggccatgcctttctagtgtctacgttgaagcagcaccctgcattccaggaactaatgccttggccacccagaaggtcacacactgcagtgcttaggctgaagcagcaacctgtatcctacagaaatggtgcctgggccactcagaacaatcacaccctctgatacataaatgcaggactttccttacagcagagaaggcctgagctgaagcaacacatcaccttctagggaatcagtgccctggcttaagctgagcagcagcacatcccaaggctgagctgatatagtaccctatgccccaggaaaacagagcagtggctaagctgagacatctcagcctacaggccaaacaactgtagtgccctgcttccctggagcttgcctaggcccctagggtctgagctgctgaggcaccctctccctgggaagtagaatcagcactgtgctgttccctgcctactccccaccaaggcccaaattacagctatgctctgccatcttgggatacttgctgacactgtacctggtctcacatagtctgggatcctgctgagccctaccatcctagggtctagagtcaataccatacagtgcctcatcccctgggaccaaagtttccattgtgccctattggctcagcttcccaaattgcagccataccatactccccaggtccacacctccagaacaccccttcttccccagagttggacaaatgctgcaccccccaggaatagactcacagctacaacctggccccttgagcaaaacctgttaaagggtacctcagagtcacagatcctagcactgtgagccacctaatccaaacctgccacagaaactgaacctacaccccaagacccagatgccacagtagattcttgaaaccaggagactaggactccagttgcacagctactctaattttctgcacctggaatccagcaccattgcagctgcacgttggccacattagacctgctaccaagagagagccccttgactaaatctcctcattgtggggaaacaagaagaggaggactacaaaagcccttgacactgagtacattaacctgtgccaccactacatccacaaacttctacagcctagtccactgaggtgcccacagttattgctgaggttgaatgcagttgaagaagacgcatggaagctatgccactgcacctattcagaaacagagtcatcacactttcccaaccaaaacactaaacccaactgcaggtgaaattgtttctctaaaaaacccactcttgaaagtttggaagaggtgactatttcgccagatgcacagacatcaatgcaggaacacaataaacatgagaaagcaaggaaatatgacaccaccaaaggaacataactctcattaatagacctcaatggaaaaagaaatcaatgtattgctggaaaaggaattcaaaataatgatcttaagataactcaataagatacaagaaaacagataaacaattcaatgaattcaggaaagtaatttacagtatgaatgaaaatttcaacagagatagaaataaaaaagaagcaaactaaaattctgcaggtgaagaattcaatgaataaaattaaaaaatacagtaatgagcttcaacagcagatatgatgaagcagaagaaaggaagagatagatcccaatacaataatagttgataacttcaacaccactctcagcactggacagataatccagacagaaagtcaacaaagaagcattcaatttaaaaagcactttagaccaaatgcatctaacagatatttacacaacatttcatccaacagctgtgaaatacacattcttttcatcagcacatggaatattctccaggataaaccatacagtaagtcacaaaacaagtctcaacaaatttgaaagaattaaaattatatcaaatatcatttatgaccacaataaaactcgaaatcaataagaggaacttttaagactgtacaaagacatgggaaattaaataatgtactcctgagtgaccaataggaattaagaagggaattttagaatgtcaacaaagaaaatgaaaatagaaatgcaacgtactagaatttataagatatagcaaaaagcagcattaagatggaagtttatggcaataaatacctacatcaaaaaagcagagagatttcaaataaatctcatccttgaaaatgcatgtcaaggaactaggaaatcaagaataaaccaaacccaaaattagtagaaagaaagaaataataaagatcagagcagaaataaatgaaattcagactaaaaaaaaaaaagatcaacaaaatgaaaaattgaatttttcaaaagataaacaaaaatgacaaataattagctagactaaacaaaaaagacccaaaagaatgaaatcagaaatgaaaacagaaacattacaactcatatcacagaaacacaaaggataattagagaccatacaccaacagattagaaaacctagcagaaacaggtatatttctggacaatacaacctaccaagactgaacccagaagaaatagaaagcctgcacaaaccagtcatgagcattgagattgaatctgtaacaaaaagtctcccatcaaaggaaagcccaaagaaattcctgttgaaataccaatgacattcctatcaaaatgccaattctacagtaatccaatggcttcagtggggaattctaccaaacactaaaaaaactaatacctattcttctgaaaatcttcccgaaaattgaagaggagggaattcttctaaattccttgtatgaggccactattatggtaattatgattatgattatgattatgtgattattattatgattccaaaaccagacaaggacacaacaaaaaaagcaaactataggctaaatactctgatgaagataatagatgaaaaccttcaaccaaatactagcaaattgaatccagcagcatattaaaaacattatttatcatgatcaagtgagatttatcccaggaatgcaaggatggttcaacatatgcaaatcacatcaacagaatgaaggacaaaaaccgtattatcatcataacagacacagaaaaagtatttgataaaatccaacatcccttcataaaaattctcagcaagttaggtatgaaaggaatatacctcaacacaataagagctatatgtgacaaacccacagccaacatcatactgaacatgcaagagttgaaagcttttctctaaaatctggaacaagccaatgatatcaggccagatgtagtggctcatgcctctaattctagcactttgggaggccaaggcaggaggatcacttgagcccaacaaaggaaacaatcaacagagtgaaaagacaatctacagaatgttagaaaatatttgcaaactctccatccaacaagggactaatatttagaatacattggaaactcaaacaactaaacagaaaagaaaacaaatttttttattaaaaataggcaaatgaaaaaatatttaaaagtttttaaaaccaaaaaaggaactgaatagacgtctctaaaaggaagatatacgaatggccaacagacatatgagaaaatgctcaacattactaatcataaggaaaatgcaaattgaaaccacaataagatataatctcaccccagttaaaatggctattattaaaaaaggcaaaaaacaattgctgtcaaagattcagagaaaggggagctttaatgcattattggttgaaatcttgtctttggaattagcacagccattgaaaaacaataaagagcttcctcaaaaaatggaaatgagtactgccccatgatcccaccatcctctaccgggtatttatccaaaggaaatctatgtcaaagagacatctacactacatgttcattgcagcactattcacaatgccagtgttggaatcaacctacgtgtccatcaacagatgaatgcataaagaaaatgtagtgtttatacacaaacacacacagagagagagagagagaggaattctattctgccatgaaaaagaatgaaatcttgtcatctgaggtaacatggatgagcttggaggacatgttaagtgaaataaagctagacacagacagccaaatatcacatgttctcactcatatatggaagccaaataagttgatctcatagaaatagggagtagaatagtggtaaccagaggctgggaatgggaggggataggatagctagaagtcgattaataaataaaaaattacagtcaggtaggaggaataggccccggtgttctctaacaccatagggtgactataactaacaacaatttattgtatattttcatacggctagaagagcaaattttgattgttcccagcacaaagaagtgataaatgtttgaggtgatggatatgctaattgttgcaggaagtcagggaccccgaatggagggaccagctggagccatggcagaggaacataaattgtgaagatttcatggacatttatcagttcccaaataatacttttataatttcttatgcctgtctttactttaatctcataatcctgttatcttcataagctgaggatgtacgtcacctcaggaccactgggataattgtgttaactgtacaaattgattgtaaaacatatgtgtttgaacaatatgaaatcagtgcaccttgaaaaagacagaataacagcgatttttagggaataagggaagacaaccataaggtctgactgcctgaggggttgggcaaaaagagccatatttttcttcttgcagaaagcctataaatgaacgtgcaagtagggaagatatcgctaaattattttcctagcaaggaatattaatactaataccctgggaaaggaatgcatccctgggggaggtctataaacggccgctctgggaatgtctgtcttatgtggttgagataaggactgagataaggactgagatacgccctggtctcctgcagtaccctcagcttattaggggggtgaaaaactccaccctggtaaatttgtggtcacactggttctctgctctcaaactctgttttctgttgtttaagatgtttatcaagataatatatgcactgctgaacatagacccttatcagtagttctgtttttgccctttgccttgtgatctttgttggacccttatcagtggttctgcttttgccctttgtcctgttccctcagaagcatgtgatctttgttagacccttattagtagttctgctttttgcctttgaagcatgtgatctttgtacccactccctgtgcttacaccccctccctttttgaaacccttaacaaaaaacctgctggtttgaggctcaggcggtcatcacagtcctactgatatgtgatgtcacccccggctgcccagctgtaaaatcctctctttatactgtctctctttatttctcagctggccaacaattatggaaaacagaaagaacctacattgaaatattgggggcaggttcccccaatactaattatcctgatttgatcatcacccattgtatatatgtatccaaatatcacaatgtaccccaaaatatatacaattattatgtgtcaattaaaaacaatcataaaacttttaaacagctaaaataaaagtatattgttttcttcaaaaaaaatctaatgcagttttccctactaggttttgggcttgcttaggacccatggctcctctctcccttccaatgtatcccttttggaataggaatgtccatcctatgcctgccccatcattgtactttggaagcagataacttcttgtcaagttacaaaggtccacagatggagaggaatttcaccccagaatgaatcaccctgcatttctcccacacttgatgcaggtgatatgtcgctgagattgtggactaagagttggtgctggaaggggttagccatcgtggagatgttgctatgggatgcagggattttgcctgtgagaaggacatgattatggggggagcggagggcaaactgtcatgggttaaaatgtgtcccctataaattcatgtgttgaagtcctaacccccaggaccacagaatgtgaccttgtttggaaacagtctttgcagctgcaatcaagttcggatgaggtcaccctggagtagggcaagcctctgatccaatatgactgctgtcctcatgaaaagggggaatctgggcacagacgcacgtgtggagaacgccctgtgaagatggtgctgcttccacaagccaagagcagcagagacggccggcaaagcccagcagcaaggagagagcctggaacagagtctccatgacacagaggagccagccccaccgagacctccatcccagatgaccggcctccagaaccaggacggaataaacgtctgttgtttaagccacgcagtctggggtgcagtgttgccagggccgcacttaacggatacgagtgttgtcctgagctgccagccccacagactgcacaaggcctccctgccccagccaagtgcagtctccccagccccctgggtgtgccatgggcagtgtggggcccatcactccgtcctcccccaggctgggaggttgagcccattatgagctccatggggtgaagctggagcgagaggctgggagccgactgggagcccgcggctggaggatggatttccccagggacccacacgtgcacctccacctgtctcctggacgttctctctgagggcagggctggtgccagctcagggatccagcagggacagaagggcgggccgggtccttgtggagagcacatttagtgggagggacatgatttcccctcacaagtgtccattcttcctgttccttgctggacgcttcctcttccattctggacacttcctgtgcgacacctcctcgggctttcccgaggccctctggcctcattccgttccctgctacctcccacttccacgtacgtccttgcccagctcttccctctatccagagcttctgcctggcaaggtccctgctgagatcagtccaggctcccccagcacaggtaggagccttgcacatgcccttggacctccccaccctgcatgatgccagcatccccaggccccagggaggccccatttctctctctgctggtagtccagtggccctggagtcccactgcaggtggggtgtgcccctgaactctgaggaagctaagtaccctgccctcagacaggctatcccccctgctcagccccagggccctgccccctaccccttcccctcacctgcaccacaggctctggccaactctgcccaggccctgaatgggcccctctggctcccctctgctgctacactgccctgcaccacctccactcagcctcagtgtgttcatccgcctgtcccacgtcccctcggcccccaggagcacagctggtggccctggctcctcgcagcccatcttgttccttctggagcaccagcctcagaggccttcctgtgcagggtccactcggccagccctgggaccctcctggtctcaagcacacacattctccctgcagccagacctgcccctgcctgtgagctcagacctgagccttggaacgccttcccttctccatcccagctcgcctttgccagctgctcagcaggatgaactcacactcccctccctgcaccatgagtgagagtcagctggagagatgcccaggcaaaagcagccaccagggcccagtgggggccagaagcttcagatgagaggcccaggtattgagaggctgagaccacgggcagaatggtcataatcgttgccagtctcagtccagccccagggactcagagacagagaaaagagcagcacacaaggtccgggctccccaccttctcccgtgagtacgggggagtatgggggcagccaccacccccatccccacacacccatgaggcagcctcagctgtgtctggactccccctcgccctctgacacagaaaccaccagaagaaaagggaacttcaggaagtaagtggtgccgccggtttcaatcctgttcttagtgtttgcagcgtggagttcacacccctggggacctgggggccgagctgtgatttcctaggaagacaagtggcagctgacagcgtgggcaaggctgcccacatgtacctcgccacaacaggaagggctgagacccccacctcggtgagtggggtcagcacagggcaggagcacaggctcaggagaaggacagagcctgggcgcagccatcggcgcttctggacctgagctgctgaacaggctgcaagaggctggggagaggctggggcgaggccagccccacatggaagcctaagcggagccagcacaggggaggtgggcagccttcaggcaccgatgcccacccagtgcgagacgacagggaccgtgggcaggggcttccaagccaacagggcaggacacaccagaggctgactgaggcctccaggacgaccgggctgggagtgtgaggaacatgacgggatggggcagagccagccatggggtgatgccaggatgggcatgaccgacctgagctcaggaggcagcagagagagggaggaggagaggccccaggtgaaccgaggggcttgtccaggccggcagcatcaccggagcccagggcagggtcagcagagctggccgtagggccctcctctcagccaggaccaaggacagcaggtgagctgggagcagagcagggagggtgagtgtggcagcaggacaggagggtggaagccaaggagcccagaggcagaggcaggg

SEQ ID NO: 3 - DNA, 2791 bp, Homo sapiens
  CDS (77)...(673)
gaaccatcattaattgaagtgagatttttctggcctgagacttgcagggaggcaagaagacactctggacaccactatggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccatgaaagaactttcaaagcctgggaagggctgcatgaaaattcagttcgtctctccagacagcttcggcgcatccttttgcccctgtatgaggttgatgacttacgagacgcatttcgtactttgggactttgatagcaacttccaggaatgtcacacacgatgaaatatctctgctgaagacagtggataaaaaacagtccttcaagtcttctctgtttttattcttcaactctcactttcttagagtttacagaaaaaatatttatatacgactctttaaaaagatctatgtcttgaaaatagagaaggaacacaggtctggccagggacgtgctgcaattggtgcagttttgaatgcaacattgtcccctactgggaataacagaactgcaggacctgggagcatcctaaagtgtcaacgtttttctatgacttttaggtaggatgagagcagaaggtagatcctaaaaagcatggtgagaggatcaaatgtttttatatcaacatcctttattatttgattcatttgagttaacagtggtgttagtgatagatttttctattcttttcccttgacgtttactttcaagtaacacaaactcttccatcaggccatgatctataggacctcctaatgagagtatctgggtgattgtgaccccaaaccatctctccaaagcattaatatccaatcatgcgctgtatgttttaatcagcagaagcatgtttttatgtttgtacaaaagaagattgttatgggtggggatggaggtatagaccatgcatggtcaccttcaagctactttaataaaggatcttaaaatgggcaggaggactgtgaacaagacaccctaataatgggttgatgtctgaagtagcaaatcttctggaaacgcaaactcttttaaggaagtccctaatttagaaacacccacaaacttcacatatcataattagcaaacaattggaaggaagttgcttgaatgttggggagaggaaaatctattggctctcgtgggtctcttcatctcagaaatgccaatcaggtcaaggtttgctacattttgtatgtgtgtgatgcttctcccaaaggtatattaactatataagagagttgtgacaaaacagaatgataaagctgcgaaccgtggcacacgctcatagttctagctgcttgggaggttgaggagggaggatggcttgaacacaggtgttcaaggccagcctgggcaacataacaagatcctgtctctcaaaaaaaaaaaaaaaaaaaagaaagagagagggccgggcgtggtggctcacgcctgtaatcccagcactttgggaggccgagccgggcggatcacctgtggtcaggagtttgagaccagcctggccaacatggcaaaaccccgtctgtactcaaaatgcaaaaattagccaggcgtggtagcaggcacctgtaatcccagctacttgggaggctgaggcaggagaatcgcttgaacccaggaggtggaggttgcagtaagctgagatcgtgccgttgcactccagcctgggcgacaagagcaagactctgtctcagaaaaaaaaaaaaaaaagagagagagagagaaagagaacaatatttgggagagaaggatggggaagcattgcaaggaaattgtgctttatccaacaaaatgtaaggagccaataagggatccctatttgtctcttttggtgtctatttgtccctaacaactgtctttgacagtgagaaaaatattcagaataaccatatccctgtgccgttattacctagcaacccttgcaatgaagatgagcagatccacaggaaaacttgaatgcacaactgtcttattttaatcttattgtacataagtttgtaaaagagttaaaaattgttacttcatgtattcatttatattttatattattttgcgtctaatgattttttattaacatgatttccttttctgatatattgaaatggagtctcaaagcttcataaatttataactttagaaatgattctaataacaacgtatgtaattgtaacattgcagtaatggtgctacgaagccatttctcttgatttttagtaaacttttatgacagcaaatttgcttctggctcactttcaatcagttaaataaatgataaataattttggaagctgtgaagataaaataccaaataaaataatataaaagtgatttatatgaagttaaaataaaaaatcagtatgatggaataaacttg

SEQ ID NO: 4 - protein, 198 aa, Homo sapiens [sequence omitted]

SEQ ID NO: 5 - DNA, 2053 bp, Homo sapiens
  CDS (71)...(1012)
cacagccacagccagggctagcctcgccggttcccgggtggcgcgcgttcgctgcctcctcagctccaggatgatcggccagaagacgctctactcctttttctcccccagccccgccaggaagcgacacgcccccagccccgagccggccgtccaggggaccggcgtggctggggtgcctgaggaaagcggagatgcggcggccatcccagccaagaaggccccggctgggcaggaggagcctgggacgccgccctcctcgccgctgagtgccgagcagttggaccggatccagaggaacaaggccgcggccctgctcagactcgcggcccgcaacgtgcccgtgggctttggagagagctggaagaagcacctcagcggggagttcgggaaaccgtattttatcaagctaatgggatttgttgcagaagaaagaaagcattacactgtttatccacccccacaccaagtcttcacctggacccagatgtgtgacataaaagatgtgaaggttgtcatcctgggacaggatccatatcatggacctaatcaagctcacgggctctgctttagtgttcaaaggcctgttccgcctccgcccagtttggagaacatttataaagagttgtctacagacatagaggattttgttcatcctggccatggagatttatctgggtgggccaagcaaggtgttctccttctcaacgctgtcctcacggttcgtgcccatcaagccaactctcataaggagcgaggctgggagcagttcactgatgcagttgtgtcctggctaaatcagaactcgaatggccttgttttcttgctctggggctcttatgctcagaagaagggcagtgccattgataggaagcggcaccatgtactacagacggctcatccctcccctttgtcagtgtatagagggttctttggatgtagacacttttcaaagaccaatgagctgctgcagaagtctggcaagaagcccattgactggaaggagctgtgatcatcagctgaggggtggcctttgagaagctgctgttaacgtatttgccagttacgaagttccactgaaaattttcctattaattcttaagtactctgcataagggggaaaagcttccagaaagcagccatgaaccaggctgtccaggaatggcagctgtatccaaccacaaacaacaaaggctaccctttgaccaaatgtctttctctgcaacatggcttcggcctaaaatatgcagaagacagatgaggtcaaatactcagttggctctctttatctcccttgcctttatggtgaaacaggggagatgtgcacctttcaggcacagccctagtttggcgcctgctgctccttggttttgcctggttagactttcagtgacagatgttggggtgtttttgcttagaaaggtccccttgtctcagccttgcagggcaggcatgccagtctctgccagttccactgcccccttgatctttgaaggagtcctcaggcccctcgcagcataaggatgttttgcaactttccagaatctggcccagaaattagggctcaatttcctgattgtagtagaggttaagattgctgtgagctttatcagataagagaccgagagaagtaagctgggtcttgttattccttgggtgttggtggaataagcagtggaatttgaacaaggaagaggagaaaagggaattttgtctttatggggtggggtgattttctcctagggttatgtccagttggggtttttaaggcagcacagactgccaagtactgttttttttaaccgactgaaatcactttgggatattttttcctgcaacactggaaagttttagttttttaagaagtactcatgcagatatatatatatatatttttcccagtcctttttttaagagacggtctttattgggtctgcacctccatccttgatcttgttagcaatgctgtttttgctgttagtcgggttagagttggctctacgcgaggtttgttaataaaagtttgttaaaagtttaaaaaaaaaaaaaaaaa

SEQ ID NO: 6 - protein, 313 aa, Homo sapiens
MIGQKTLYSFFSPSPARKRHAPSPEPAVQGTGVAGVPEESGDAAAIPAKKAPAGQEEPGTPPSSPLSAEQLDRIQRNKAAALLRLAARNVPVGFGESWKKHLSGEFGKPYFIKLMGFVAEERKHYTVYPPPHQVFTWTQMCDIKDVKVVILGQDPYHGPNQAHGLCFSVQRPVPPPPSLENIYKELSTDIEDFVHPGHGDLSGWAKQGVLLLNAVLTVRAHQANSHKERGWEQFTDAVVSWLNQNSNGLVFLLWGSYAQKKGSAIDRKRHHVLQTAHPSPLSVYRGFFGCRHFSKTNELLQKSGKKPIDWKEL

SEQ ID NO: 7 - DNA, 2156 bp, Homo sapiens
  CDS (71)...(1900)
gcgcatgcgtggattgtcgtcttctgtccaagttggtcgcttccctgcgccaaagtgagcagtagccaacatgtcagggtgggagtcatattacaaaaccgagggcgatgaagaagcagaggaagaacaagaagagaaccttgaagcaagtggagactataaatattcaggaagagatagtttgatttttttggttgatgcctccaaggctatgtttgaatctcagagtgaagatgagttgacaccttttgacatgagcatccagtgtatccaaagtgtgtacatcagtaagatcataagcagtgatcgagatctcttggctgtggtgttctatggtaccgagaaagacaaaaattcagtgaattttaaaaatatttacgtcttacaggagctggataatccaggtgcaaaacgaattctagagcttgaccagtttaaggggcagcagggacaaaaacgtttccaagacatgatgggccacggatctgactactcactcagtgaagtgctgtgggtctgtgccaacctctttagtgatgtccaattcaagatgagtcataagaggatcatgctgttcaccaatgaagacaacccccatggcaatgacagtgccaaagccagccgggccaggaccaaagccggtgatctccgagatacaggcatcttccttgacttgatgcacctgaagaaacctgggggctttgacatatccttgttctacagagatatcatcagcatagcagaggatgaggacctcagggttcactttgaggaatccagcaagctagaagacctgttgcggaaggttcgcgccaaggagaccaggaagcgagcactcagcaggttaaagctgaagctcaacaaagatatagtgatctctgtgggcatttataatctggtccagaaggctctcaagcctcctccaataaagctctatcgggaaacaaatgaaccagtgaaaaccaagacccggacctttaatacaagtacaggcggtttgcttctgcctagcgataccaagaggtctcagatctatgggagtcgtcagattatactggagaaagaggaaacagaagagctaaaacggtttgatgatccaggtttgatgctcatgggtttcaagccgttggtactgctgaagaaacaccattacctgaggccctccctgttcgtgtacccagaggagtcgctggtgattgggagctcaaccctgttcagtgctctgctcatcaagtgtctggagaaggaggttgcagcattgtgcagatacacaccccgcaggaacatccctccttattttgtggctttggtgccacaggaagaagagttggatgaccagaaaattcaggtgactcctccaggcttccagctggtctttttaccctttgctgatgataaaaggaagatgccctttactgaaaaaatcatggcaactccagagcaggtgggcaagatgaaggctatcgttgagaagcttcgcttcacatacagaagtgacagctttgagaaccccgtgctgcagcagcacttcaggaacctggaggccttggccttggatttgatggagccggaacaagcagtggacctgacattgcccaaggttgaagcaatgaataaaagactgggctccttggtggatgagtttaaggagcttgtttacccaccagattacaatcctgaagggaaagttaccaagagaaaacacgataatgaaggttctggaagcaaaaggcccaaggtggagtattcagaagaggagctgaagacccacatcagcaagggtacgctgggcaagttcactgtgcccatgctgaaagaggcctgccgggcttacgggctgaagagtgggctgaagaagcaggagctgctggaagccctcaccaagcacttccaggactgaccagaggccgcgcgtccagctgcccttccgcagtgtggccaggctgcctggccttgtcctcagccagttaaaatgtgtttctcctgagctaggaagagtctacccgacataagtcgagggactttatgtttttgaggctttctgttgccatggtgatggtgtagccctcccactttgctgttccttactttactgcctgaataaagagccctaagtttgtactatatactgttaaaaaaaaaaaaaaaaaaaaaaa

SEQ ID NO: 8 - protein, 609 aa, Homo sapiens
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQD

SEQ ID NO: 9 - DNA, 3310 bp, Homo sapiens
  CDS (34)...(2232)
ggcgggcgaccaaagcgcctgaggaccggcaacatggtgcggtcggggaataaggcagctgttgtgctgtgtatggacgtgggctttaccatgagtaactccattcctggtatagaatccccatttgaacaagcaaagaaggtgataaccatgtttgtacagcgacaggtgtttgctgagaacaaggatgagattgctttagtcctgtttggtacagatggcactgacaatcccctttctggtggggatcagtatcagaacatcacagtgcacagacatctgatgctaccagattttgatttgctggaggacattgaaagcaaaatccaaccaggttctcaacaggctgacttcctggatgcactaatcgtgagcatggatgtgattcaacatgaaacaataggaaagaagtttgagaagaggcatattgaaatattcactgacctcagcagccgattcagcaaaagtcagctggatattataattcatagcttgaagaaatgtgacatctccctgcaattcttcttgcctttctcacttggcaaggaagatggaagtggggacagaggagatggcccctttcgcttaggtggccatgggccttcctttccactaaaaggaattaccgaacagcaaaaagaaggtcttgagatagtgaaaatggtgatgatatctttagaaggtgaagatgggttggatgaaatttattcattcagtgagagtctgagaaaactgtgcgtcttcaagaaaattgagaggcattccattcactggccctgccgactgaccattggctccaatttgtctataaggattgcagcctataaatcgattctacaggagagagttaaaaagacttggacagttgtggatgcaaaaaccctaaaaaaagaagatatacaaaaagaaacagtttattgcttaaatgatgatgatgaaactgaagttttaaaagaggatattattcaagggttccgctatggaagtgatatagttcctttctctaaagtggatgaggaacaaatgaaatataaatcggaggggaagtgcttctctgttttgggattttgtaaatcttctcaggttcagagaagattcttcatgggaaatcaagttctaaaggtctttgcagcaagagatgatgaggcagctgcagttgcactttcctccctgattcatgctttggatgacttagacatggtggccatagttcgatatgcttatgacaaaagagctaatcctcaagtcggcgtggcttttcctcatatcaagcataactatgagtgtttagtgtatgtgcagctgcctttcatggaagacttgcggcaatacatgttttcatccttgaaaaacagtaagaaatatgctcccaccgaggcacagttgaatgctgttgatgctttgattgactccatgagcttggcaaagaaagatgagaagacagacacccttgaagacttgtttccaaccaccaaaatcccaaatcctcgatttcagagattatttcagtgtctgctgcacagagctttacatccccgggagcctctacccccaattcagcagcatatttggaatatgctgaatcctcccgctgaggtgacaacaaaaagtcagattcctctctctaaaataaagaccctttttcctctgattgaagccaagaaaaaggatcaagtgactgctcaggaaattttccaagacaaccatgaagatggacctacagctaaaaaattaaagactgagcaagggggagcccacttcagcgtctccagtctggctgaaggcagtgtcacctctgttggaagtgtgaatcctgctgaaaacttccgtgttctagtgaaacagaagaaggccagctttgaggaagcgagtaaccagctcataaatcacatcgaacagtttttggatactaatgaaacaccgtattttatgaagagcatagactgcatccgagccttccgggaagaagccattaagttttcagaagagcagcgctttaacaacttcctgaaagcccttcaagagaaagtggaaattaaacaattaaatcatttctgggaaattgttgtccaggatggaattactctgatcaccaaagaggaagcctctggaagttctgtcacagctgaggaagccaaaaagtttctggcccccaaagacaaaccaagtggagacacagcagctgtatttgaagaaggtggtgatgtggacgatttattggacatgatataggtcgtggatgtatggggaatctaagagagctgccatcgctgtgatgctgggagttctaacaaaacaagttggatgcggccattcaaggggagccaaaatctcaagaaattcccagcaggttacctggaggcggatcatctaattctctgtggaatgaatacacacatatatattacaagggataatttagaccccatacaagtttataaagagtcattgttattttctggttggtgtattattttttctgtggtcttactgatctttgtatattacatacatgctttgaagtttctggaaagtagatcttttcttgacctagtatatcagtgacagttgcagcccttgtgatgtgattagtgtctcatgtggaaccatggcatggttattgatgagtttcttaaccctttccagagtcctcctttgcctgatcctccaacagctgtcacaacttgtgttgagcaagcagtagcatttgcttcctcccaacaagcagctgggttaggaaaaccatgggtaaggacggactcacttctctttttagttgaggccttctagttaccacattactctgcctctgtatataggtggttttctttaagtggggtgggaaggggagcacaatttcccttcatactccttttaagcagtgagttatggtggtggtctcatgaagaaaagaccttttggcccaatctctgccatatcagtgaacctttagaaactcaaaaactgagaaatttacttcagtagttagaattatatcacttcactgttctctacttgcaagcctcaaagagagaaagtttcgttatattaaaacacttaggtaacttttcggtctttcccatttctacctaagtcagctttcatctttgtggatggtgtctcctttactaaataagaaaataacaaagcccttattctctttttttcttgtcctcattcttgccttgagttccagttcctctttggtgtacagacttcttggtacccagtcacctctgtcttcagcaccctcataagtcgtcactaatacacagttttgtacatgtaacattaaaggcataaatgactc

SEQ ID NO: 10 - protein, 732 aa, Homo sapiens
MVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISLQFFLPFSLGKEDGSGDRGDGPFRLGGHGPSFPLKGITEQQKEGLEIVKMVMISLEGEDGLDEIYSFSESLRKLCVFKKIERHSIHWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAKKLKTEQGGAHFSVSSLAEGSVTSVGSVNPAENFRVLVKQKKASFEEASNQLINHIEQFLDTNETPYFMKSIDCIRAFREEAIKFSEEQRFNNFLKALQEKVEIKQLNHFWEIVVQDGITLITKEEASGSSVTAEEAKKFLAPKDKPSGDTAAVFEEGGDVDDLLDMI

SEQ ID NO: 11 - DNA, 6582 bp, Homo sapiens
  CDS (125)...(3256)
agagggcaaggagagagcagagaacacactttgccttctctttggtattgagtaatatcaaccaaattgcagacatctcaacactttggccaggcagcctgctgagcaaggtacctcagccagcatggcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggccggccccgccaacatcttctgtcgctgactcggagagctcagaagcaccggctgagggagctcaagctgcaagtcaaagcctttgctgacaaagaagaaggtggagatgtgaagtccgtgtgcatgaccttgttcctgctggctctgagggcgaggaatgagcacaggcaagctgatgagctggaggccatcatgcagggaaagggctctggcctgcagccagctgtttgcttggccatccgtgtcaacaccttcctcagctgcagtcagtaccacaagatgtacaggactgtgaaagccatcacagggagacagatttttcagcctttgcatgcccttcggaatgctgagaaggtacttctgccaggctaccaccactttgagtggcagccacctctgaagaatgtgtcttccagcactgatgttggcattattgatgggctgtctggactatcatcctctgtggatgattacccagtggacaccattgcaaagaggttccgctatgattcagctttggtgtctgctttgatggacatggaagaagacatcttggaaggcatgagatcccaagaccttgatgattacctgaatggccccttcactgtggtggtgaaggagtcttgtgatggaatgggagacgtgagtgagaagcatgggagtgggcctgtagttccagaaaaggcagtccgtttttcattcacaatcatgaaaattactattgcccacagctctcagaatgtgaaagtatttgaagaagccaaacctaactctgaactgtgttgcaagccattgtgccttatgctggcagatgagtctgaccacgagacgctgactgccatcctgagtcctctcattgctgagagggaggccatgaagagcagtgaattaatgcttgagctgggaggcattctccggactttcaagttcatcttcaggggcaccggctatgatgaaaaacttgtgcgggaagtggaaggcctcgaggcttctggctcagtctacatttgtactctttgtgatgccacccgtctggaagcctctcaaaatcttgtcttccactctataaccagaagccatgctgagaacctggaacgttatgaggtctggcgttccaacccttaccatgagtctgtggaagaactgcgggatcgggtgaaaggggtctcagctaaacctttcattgagacagtcccttccatagatgcactccactgtgacattggcaatgcagctgagttctacaagatcttccagctagagataggggaagtgtataagaatcccaatgcttccaaagaggaaaggaaaaggtggcaggccacactggacaagcatctccggaagaagatgaacctcaaaccaatcatgaggatgaatggcaactttgccaggaagctcatgaccaaagagactgtggatgcagtttgtgagttaattccttccgaggagaggcacgaggctctgagggagctgatggatctttacctgaagatgaaaccagtatggcgatcatcatgccctgctaaagagtgcccagaatccctctgccagtacagtttcaattcacagcgttttgctgagctcctttctacgaagttcaagtataggtatgagggaaaaatcaccaattattttcacaaaaccctggcccatgttcctgaaattattgagagggatggctccattggggcatgggcaagtgagggaaatgagtctggtaacaaactgtttaggcgcttccggaaaatgaatgccaggcagtccaaatgctatgagatggaagatgtcctgaaacaccactggttgtacacctccaaatacctccagaagtttatgaatgctcataatgcattaaaaacctctgggtttaccatgaaccctcaggcaagcttaggggacccattaggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagttgtcaaaagtgcatcggcgacattatctttaattgtatgtatttggtgcttcttcagggattgaactcagtatctttcattaaaaaacacagcagttttccttgctttttatatgcagaatatcaaagtcatttctaatttagttgtcaaaaacatatacatattttaacattagtttttttgaaaactcttggttttgtttttttggaaatgagtgggccactaagccacactttcccttcatcctgcttaatccttccagcatgtctctgcactaataaacagctaaattcacataatcatcctatttactgaagcatggtcatgctggtttatagattttttacccatttctactctttttctctattggtggcactgtaaatactttccagtattaaattatccttttctaacactgtaggaactattttgaatgcatgtgactaagagcatgatttatagcacaacctttccaataatcccttaatcagatcacattttgataaaccctgggaacatctggctgcaggaatttcaatatgtagaaacgctgcctatggttttttgcccttactgttgagactgcaatatcctagaccctagttttatactagagttttatttttagcaatgcctattgcaagtgcaattatatactccagggaaattcaccacactgaatcgagcatttgtgtgtgtatgtgtgaagtatatactgggacttcagaagtgcaatgtatttttctcctgtgaaacctgaatctacaagttttcctgccaagccactcaggtgcattgcagggaccagtgataatggctgatgaaaattgatgattggtcagtgaggtcaaaaggagccttgggattaataaacatgcactgagaagcaagaggaggagaaaaagatgtctttttcttccaggtgaactggaatttagttttgcctcagatttttttcccacaagatacagaagaagataaagatttttttggttgagagtgtgggtcttgcattacatcaaacagagttcaaattccacacagataagaggcaggatatataagcgccagtggtagttgggaggaataaaccattatttggatgcaggtggtttttgattgcaaatatgtgtgtgtcttcagtgattgtatgacagatgatgtattcttttgatgttaaaagattttaagtaagagtagatacattgtacccattttacattttcttattttaactacagtaatctacataaatatacctcagaaatcatttttggtgattattttttgttttgtagaattgcacttcagtttattttcttacaaataaccttacattttgtttaatggcttccaagagccttttttttttttgtatttcagagaaaattcaggtaccaggatgcaatggatttatttgattcaggggacctgtgtttccatgtcaaatgttttcaaataaaatgaaatatgagtttcaatactttttatattttaatatttccattcattaatattatggttattgtcagcaattttatgtttgaatatttgaaataaaagtttaagatttgaaaa

SEQ ID NO: 12 - protein, 1043 aa, Homo sapiens
MAASFPPTLGLSSAPDEIQHPHIKFSEWKFKLFRVRSFEKTPEEAQKEKKDSFEGKPSLEQSPAVLDKADGQKPVPTQPLLKAHPKFSKKFHDNEKARGKAIHQANLRHLCRICGNSFRADEHNRRYPVHGPVDGKTLGLLRKKEKRATSWPDLIAKVFRIDVKADVDSIHPTEFCHNCWSIMHRKFSSAPCEVYFPRNVTMEWHPHTPSCDICNTARRGLKRKSLQPNLQLSKKLKTVLDQARQARQRKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDPLGIEDSLESQDSMEF

SEQ ID NO: 13 - DNA, 967 bp, Homo sapiens
  CDS (30)...(743)
gcagaagtctctctcagtcaggacacagcatggacatgagggtccctgctcagctcctgggactcctgctgctctggctcccagataccagatgtgacatccagatgacccagtctccatcctccctgtctgcatctgtaggagacagagtcaccatcacttgccgggcgaatcaggacattaagaattatgtagcctggtatcagcagaaaccagggaaagttcctaaggtcctgatctatgctgcatccactttgcaatcgggggtcccatctcggttcagtggctctggatctgggacagatttcactctcaccatcaccagcctgcagcctgaagatgttgcaacttattactgtcaaacatataccagtgcccccccttggacgttcggccaagggaccaaggtggagatcaatcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcaaaaaaaaaaaaaaaaaaaaaaaaaaaaaa

SEQ ID NO: 14 - protein, 237 aa, Homo sapiens
MDMRVPAQLLGLLLLWLPDTRCDIQMTQSPSSLSASVGDRVTITCRANQDIKNYVAWYQQKPGKVPKVLIYAASTLQSGVPSRFSGSGSGTDFTLTITSLQPEDVATYYCQTYTSAPPWTFGQGTKVEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 899 bp, Homo sapiens
  CDS (322)...(741)
agatgcccctctgggagagatccccaggggtgacagccatggaccctggaagggcctgggctagggacagggaccagagccagtccagggagaggacagagccaatggactggggtgtactgtaacagccctgctggcgagagggaccagggcaccgtcctccagggagcccatgctgcaagtcgggccagaggtgcccctgaacctgaaggccaatgagacccaagacaggccaagtgggttgtgagacccctgaggagctgggccctggtcccaggcagcgctggcccctgctgctgctgggtctggccatggtcgcccatggcctgctgcgcccaatggttgcaccgcaaagcggggacccagaccctggagcctcagttggaagcagccgatccagcctgcggagcctgtggggcaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcataggttctcaaccctcacccccaccacgggagactagagctgcaggatcccaggggaggggtctctcctcccaccccaaggcatcaagcccttctccctgcactcaataaaccctcaataaatattctcattgtcaatcaaaaaaaaaaaaaaaaaaaaaa

SEQ ID NO: 16 - protein, 139 aa, Homo sapiens
MACCAQWLHRKAGTQTLEPQLEAADPACGACGAGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 17 - DNA, 1869 bp, Homo sapiens
tacctggttgatcctgccagtagcatatgcttgtctcaaagattaagccatgcatgtctaagtacgcacggccggtacagtgaaactgcgaatggctcattaaatcagttatggttcctttggtcgctcgctcctctcccacttggataactgtggtaattctagagctaatacatgccgacgggcgctgacccccttcgcgggggggatgcgtgcatttatcagatcaaaaccaacccggtcagcccctctccggccccggccggggggcgggcgccggcggctttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagccaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggaggttcgaagacgatcagataccgtcgtagttccgaccataaacgatgccgaccggcgatgcggcggcgttattcccatgacccgccgggcagcttccgggaaaccaaagtctttgggttccggggggagtatggttgcaaagctgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatggtttagtgaggccctcggatcggccccgccggggtcggcccacggccctggcggagcgctgagaagacggtcgaacttgactatctagaggaagtaaaagtcgtaacaaggtttccgtaggtgaacctgcggaaggatcatta

SEQ ID NO: 18 - DNA, 987 bp, Homo sapiens
  CDS (61)...(420)
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 19 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 20 - DNA, 1704 bp, Homo sapiens
cctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagtgccacggccggcaagcccccgctccccaggctctcggggtcgcgcgaggatgcttggcacgtaccccgtgtacatacttcccaggcacccagcatggaaataaa

SEQ ID NO: 21 - DNA, 1527 bp, Homo sapiens
catccccgaccagccccaaggtcttcccgctgagcctctgcagcacccagccagatgggaacgtggtcatcgcctgcctggtccagggcttcttcccccaggagccactcagtgtgacctggagcgaaagcggacagggcgtgaccgccagaaacttcccacccagccaggatgcctccggggacctgtacaccacgagcagccagctgaccctgccggccacacagtgcctagccggcaagtccgtgacatgccacgtgaagcactacacgaatcccagccaggatgtgactgtgccctgcccaggtcagagggcaggctggggagtggggcggggccaccccgtcgtgccctgacactgcgcctgcacccgtgttccccacagggagccgccccttcactcacaccagagtggaccgcgggccgagccccaggaggtggtggtggacaggccaggaggggcgaggcgggggcatggggaagtatgtgctgaccagctcaggccatctctccactccagttccctcaactccacctaccccatctccctcaactccacctaccccatctccctcatgctgccacccccgactgtcactgcaccgaccggccctcgaggacctgctcttaggttcagaagcgaacctcacgtgcacactgaccggcctgagagatgcctcaggtgtcaccttcacctggacgccctcaagtgggaagagcgctgttcaaggaccacctgagcgtgacctctgtggctgctacagcgtgtccagtgtcctgccgggctgtgccgagccatggaaccatgggaagaccttcacttgcactgctgcctaccccgagtccaagaccccgctaaccgccaccctctcaaaatccggtgggtccagaccctgctcggggccctgctcagtgctctggtttgcaaagcatattcctggcctgcctcctccctcccaatcctgggctccagtgctcatgccaagtacagagggaaactgaggcaggctgaggggccaggacacagcccagggtgcccaccagagcagaggggctctctcatcccctgcccagccccctgacctggctctctaccctccaggaaacacattccggcccgaggtccacctgctgccgccgccgtcggaggagctggccctgaacgagctggtgacgctgacgtgcctggcacgcggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctactgagccgcccgcctgtccccacccctgaataaa

SEQ ID NO: 22 - DNA, 21 bp, Artificial Sequence
  Forward primer
ctctggccctgcttattgttg

SEQ ID NO: 23 - DNA, 24 bp, Artificial Sequence
  Reverse primer
gaagacatttgggaaggactgact

SEQ ID NO: 24 - DNA, 20 bp, Artificial Sequence
  Forward primer
tgggcaagtggatctgaaca

SEQ ID NO: 25 - DNA, 22 bp, Artificial Sequence
  Reverse primer
ctcagggaagtagcctttgaca

SEQ ID NO: 26 - DNA, 20 bp, Artificial Sequence
  Foward primer
ggcccttccagatctttgag

SEQ ID NO: 27 - DNA, 22 bp, Artificial Sequence
  Reverse primer
ggatccagagttccaggtcact

SEQ ID NO: 28 - DNA, 27 bp, Artificial Sequence
  Forward primer
gggcttccaagccaacagggcaggaca

SEQ ID NO: 29 - DNA, 21 bp, Artificial Sequence
  Reverse primer
gttgccgttggggtgctggac

SEQ ID NO: 30 - DNA, 25 bp, Artificial Sequence
  Forward primer
ggctgaggttagggttccatctcag

SEQ ID NO: 31 - DNA, 26 bp, Artificial Sequence
  Reverse primer
gagggagtcaagaaagtcacgctgga

SEQ ID NO: 32 - DNA, 26 bp, Artificial Sequence
  Forward primer
gttagcatgccagagtctcgttcgtt

SEQ ID NO: 33 - DNA, 17 bp, Artificial Sequence
  Reverse primer
ccgttcttagttggtgg

SEQ ID NO: 34 - DNA, 25 bp, Artificial Sequence
  Forward primer
cctgaattatttcagttaagcatgt

SEQ ID NO: 35 - DNA, 19 bp, Artificial Sequence
  Reverse primer
cctcgtctcctgtgagaat

SEQ ID NO: 36 - DNA, 16 bp, Artificial Sequence
  nucleic acid probe
ctccatcactttctcc

SEQ ID NO: 37 - DNA, 16 bp, Artificial Sequence
  Forward primer
caacagggcaggacac

SEQ ID NO: 38 - DNA, 20 bp, Artificial Sequence
  Forward primer
gaggatctcctgtccatcag

SEQ ID NO: 39 - DNA, 25 bp, Artificial Sequence
  Reverse primer
ccaaatgtggccgtggtttctgtca

SEQ ID NO: 40 - DNA, 18 bp, Artificial Sequence
  Nucleic acid probe
tcacctctcctactcact

SEQ ID NO: 41 - DNA, 25 bp, Artificial Sequence
  Forward primer
ctagaaattgacacaagtggacctt

SEQ ID NO: 42 - DNA, 25 bp, Artificial Sequence
  Reverse primer
cccggtctcccaaataaatacattc

SEQ ID NO: 43 - DNA, 15 bp, Artificial Sequence
  Nucleic acid probe
gccttcctctctcca

SEQ ID NO: 44 - DNA, 24 bp, Artificial Sequence
  Forward Primer
ctgtgactttggcttatctgatct

SEQ ID NO: 45 - DNA, 25 bp, Artificial Sequence
  Reverse Primer
gcgtcactttgaagaatctcctggt

SEQ ID NO: 46 - DNA, 17 bp, Artificial Sequence
  Nucleic acid probe
gcctgtgttcccttgtg

SEQ ID NO: 47 - DNA, 23 bp, Artificial Sequence
  Forward primer
ccaatgaaaggccctattgctat

SEQ ID NO: 48 - DNA, 25 bp, Artificial Sequence
  Reverse primer
cagtgaagacatcctcctgaagagt

SEQ ID NO: 49 - DNA, 15 bp, Artificial Sequence
  Nucleic acid probe
aatctggtccaaaac

SEQ ID NO: 50 - DNA, 28 bp, Artificial Sequence
  Forward primer
gacacggacaggattgacagattgatag

SEQ ID NO: 51 - DNA, 26 bp, Artificial Sequence
  Reverse primer
gttagcatgccagagtctcgttcgtt

SEQ ID NO: 52 - DNA, 17 bp, Artificial Sequence
  Nucleic acid probe
ccgttcttagttggtgg

SEQ ID NO: 53 - DNA, 24 bp, Artificial Sequence
  Forward primer
acagtcttagggagagtttatgac

SEQ ID NO: 54 - DNA, 19 bp, Artificial Sequence
  Reverse primer
caccacgtgttcgtctgtg

SEQ ID NO: 55 - DNA, 25 bp, Artificial Sequence
  Forward primer
gatattctgatagagtggccttcat

SEQ ID NO: 56 - DNA, 16 bp, Artificial Sequence
  Forward primer
agcagagctggccgta

SEQ ID NO: 57 - DNA, 17 bp, Artificial Sequence
  Forward primer
ccagaaaggcccagagt

SEQ ID NO: 58 - DNA, 480 bp, Artificial Sequence
  Nucleotides 961381 to 961860 of GenBank Accession No. NG_001019 (Jan 10, 2006 version).
gtaagatgtttaagaaattaaacagtcttagggagagtttatgactgtattcaaaaagttttttaaattagcttgttatcccttcatgtgataactaatctcaaatactttttcgatacctcagagcattattttcataatgactgtgttcacaatctttttaggttaactcgttttctctttgtgattaaggagaaacactttgatattctgatagagtggccttcattttagtatttttcaagaccacttttcaactactcactttaggataagttttaggtaaaatgtgcatcattatcctgaattatttcagttaagcatgttagttggtggcataagagaaaactcaatcagatagtgctgaagacaggactgtggagacaccttagaaggacagattctgttccgaatcaccgatgcggcgtcagcaggactggcctagcggaggctctgggagggtggctgccaggcccggcc

SEQ ID NO: 59 - DNA, 360 bp, Artificial Sequence
  Nucleotides 967201 to 967560 of GenBank Accession No. NG_001019 (Jan 10, 2006 version).
tgtcctgcgggtcctcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttccaggtgagggccgggcccagccaccgggacaga

SEQ ID NO: 60 - DNA, 421 bp, Artificial Sequence
  Nucleotides 1047841 to 1048261 of GenBank Accession No. NG_001019 (Jan 10, 2006 version).
ccttcaggcaccgatgcccacccagtgcgagacgacagggaccgtgggcaggggcttccaagccaacagggcaggacacaccagaggctgactgaggcctccaggacgaccgggctgggagtgtgaggaacatgacgggatggggcagagccagccatggggtgatgccaggatgggcatgaccgacctgagctcaggaggcagcagagagagggaggaggagaggccccaggtgaaccgaggggcttgtccaggccggcagcatcaccggagcccagggcagggtcagcagagctggccgtagggccctcctctcagccaggaccaaggacagcaggtgagctgggagcagagcagggagggtgagtgtggcagcaggacaggagggtggaagccaaggagcccagaggcagaggcaggg

(19) United States
(12) Patent Application Publication
(10) Pub. No.: US 2016/0251716 A1
(43) Pub. Date: Sep. 1, 2016

(54) METHODS AND KITS FOR MONITORING THE EFFECTS OF IMMUNOMODULATORS ON ADAPTIVE IMMUNITY

(71) Applicant: Millennium Pharmaceuticals, Inc., Cambridge, MA (US)

(72) Inventor: Eric R. FEDYK, Lake Forest, IL (US)

(73) Assignee: Millennium Pharmaceuticals, Inc.

(21) Appl. No.: 14/872,477

(22) Filed: Oct. 1, 2015

Related U.S. Application Data
(63) Continuation of application No. 12/991,994, filed on Nov. 10, 2010, now abandoned.
(60) Provisional application No. 61/127,522, filed on May 14, 2008.

(51) Int. Cl. C12Q 1/68; G06Q 50/22; G06Q 20/40

(52) U.S. Cl. CPC C12Q 1/6883; G06Q 20/40; G06Q 50/22; C12Q 2600/118; C12Q 2600/106; C12Q 2600/158

(57) ABSTRACT

The invention provides for noninvasive assessment of immunocompetence in various situations, for example, when modified by disease or by immunomodulators. The assessment determines the functional activity of germinal centers via measuring levels of immunogolublin isotype class switching. The invention provides for assessment of therapeutic efficacy of immunomodulators and for selection of treatment regimens. The invention also provides for determining the risk or susceptibility to adverse events upon receipt of therapy. Compositions, kits and methods are described herein.

### CROSS REFERENCE TO RELATED APPLICATION

This application claims the benefit of U.S. Provisional Application No. 61/127,522, filed May 14, 2008, the entire contents of which are incorporated herein.

### BACKGROUND OF THE INVENTION

Germinal centers are unique substructures of follicles within secondary lymphoid organs (e.g., spleen, lymph node, gut-associated lymphoid tissue, etc.) and chronically inflammed tissues (e.g., rheumatoid synovium). Functional activity within germinal centers is important for protective immunity of mammalian species. Biologic activities driven by germinal centers that are important for generating efficient antibody responses include clonal expansion of B lymphocytes, immunoglobulin isotype class switching and somatic mutation of immunoglobulin genes.

The germinal centers of secondary lymphoid organs generate high affinity antibody responses to antigens and provide protective immunity against pathogens. Germinal centers also generate autoantibodies that drive the pathogenesis of some autoimmune disease. Many immunosuppressive drugs decrease the activity of germinal centers (i.e., cause atrophy) and inhibit antibody responses. These drugs thus could have efficacy for treating autoimmune disease and/or predispose subjects to infection, depending on the magnitude of immunosuppression. Conversely, stimulation of the immune system, for example, by vaccination, increases the activity of germinal centers and generates protective antibody responses. The convention for assessing the activity of germinal centers is assessment of their morphology in tissue sections from secondary lymphoid organs, by an anatomic pathologist. This procedure, which requires obtaining tissue from secondary lymphoid organs, is an invasive procedure that causes discomfort and risk to the patient. An alternative is a human vaccination study, within which subjects are immunized with a specific antigen and the antibody response to this antigen is monitored via serum samples. The major risk of this approach is the stimulation of a subject's immune system, which can cause adverse events ranging from minor (i.e., fever and malaise) to severe (i.e., death). A safer, less tedious and less invasive methodology of assessing immune competence is preferred by clinicians. A routine, noninvasive test for germinal center activity can identify patients at risk for developing adverse conditions and prompt prophylactic measures that prevent the condition, to avoid the morbidity and cost associated with treating the condition.

### SUMMARY OF THE INVENTION

The invention relates to assessment of immunocompetence of the adaptive immune system by noninvasive measurement of follicular germinal center activity. The activity of germinal centers correlates positively with antibody responses to antigens in vaccination studies. The activity of germinal centers correlates inversely with adverse events caused by therapeutic agents. For example, splenic germinal center atrophy can lead to increased susceptibility to pathogens, e.g., bacterial infection or cancer, e.g., lymphoma. In another example, germinal center hyperplasia can indicate efficacy of vaccination to enhance immunity.

In one aspect, the invention provides compositions and kits useful in determination of germinal center activity. In another aspect, the invention provides methods for determining germinal center activity. In these aspects, biomarkers in patient samples are measured. In one embodiment, the activity is increased (i.e., due to hyperplasia). In this embodiment an increase in the level of biomarker can be measured. In another embodiment, the activity is decreased (i.e., due to atrophy). In this embodiment, a decrease in the level of biomarker can be measured.

In some embodiments, the biomarkers allow measurement of therapeutic activity of agents administered to treat pathogenic conditions and disease.

In other embodiments, the biomarkers allow measurement of the efficacy of vaccination, e.g., to prevent disease.

In other embodiments, the biomarkers are predictive of drug-induced splenic germinal center atrophy.

In other embodiments, patient risk for detrimental immunosuppression can be monitored by analysis of differences or trends in germinal center activity over time. In another embodiment, the biomarkers allow the selection of treatment regimen for a patient.

In further embodiments, the invention relates to stimulation of germinal center activity and thus gain of immunity for protection from pathogenic disorders, e.g., after vaccination.

Biomarkers preferably are measured in peripheral blood samples. Preferred biomarkers include germline transcript mu (GLT-μ), activation-induced cytidine deaminase (AID), circular transcripts containing gamma 1 and 2 (CT-γ1&2), immunoglobulin heavy chain locus G1 isotype (IGHG1) and/or immunoglobulin heavy chain locus A1 isotype (IGHA1).

Preferred methods analyze nucleic acid transcripts of the biomarkers.

Other features and advantages of the invention will be apparent from the following detailed description, and from the claims.

### BRIEF DESCRIPTION OF THE DRAWINGS

FIG. 1. Effect of ML120B on switch transcripts in mouse spleens after a single dose.

FIG. 2. Effect of ML120B on switch transcripts in mouse spleens after a 6 hour treatment.

FIG. 3. Effect of ML120B on switch transcripts in mouse spleens for 4 days of treatments.

FIG. 4. Histopathology of secondary lymphoid organs (spleen, mandibular lymph node (Mand. LN), popliteal lymph node (pop. LN) and mesenteric lymph node (Mes. LN)) of cynomolgous monkeys in study using Test Agent A.

FIG. 5. Frequency of IgG+ germinal centers in spleens of cynomolgous monkeys in study using Test Agent A.

FIGS. 6A-C. Flow cytometry of peripheral blood from cynomolgous monkeys in study using Test Agent A. 6A, Total B cells; 6B, percent change in number of post-germinal center B cells; 6C, percent change in number of pre-germinal center B cells.

FIGS. 7A-D. Fold change of GLT-μ (ST1) transcript in blood over time in cynomolgous monkey study using Test Agent A (TA). FIG. 7A, vehicle-treated animals; FIG. 7B, 40 mg/kg-treated animals; FIG. 7C, 60 mg/kg-treated animals; FIG. 7D, 100 mg/kg-treated animals.

FIGS. 8A-B. Measurement of ICS transcripts in blood from normal human volunteers. FIG. 8A, level of GLT-μ; FIG. 8B, level of circle transcripts CT-γ1&2.

FIG. 9. Measurement of ICS transcripts (circle transcripts CT-γ1&2) in human blood samples, unmodified or modified to remove non-B cell populations.

FIG. 10. Comparison of timecourse of treatment of cynomolgous monkeys with Test Agent A with timecourse of contracting bacterial infection.

FIGS. 11A-B. Fold change of transcripts in peripheral blood of cynomolgous monkeys during timecourse of study using Test Agent A. FIG. 11A, animal No. 1005 (vehicle control); FIG. 11B, animal No. 4005 (80 mg/kg Test Agent A for 13 weeks from Day 0).

FIGS. 12 A-H. Correlation of fold change from baseline of level of transcripts with histological assessment of reactivity of germinal centers in study of using Test Agent A in cynomolgous monkeys. FIG. 12A, correlation of measurement of GLT-μ; FIG. 12B, correlation of measurement of CT-γ1&2; FIG. 12C, correlation of measurement of AID; FIG. 12D, correlation of measurement of 18S RNA; FIG. 12E, correlation of measurement of IGHG1; FIG. 12F, correlation of measurement of IGHA1; FIG. 12G, correlation of measurement of IGL-κ; FIG. 12H, correlation of measurement of IGL-λ.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, preferred methods and materials are described herein. The content of all database accession records (e.g., sequences associated with representative public identifier ID, e.g., Entrez, GenBank, RefSeq) cited throughout this application are hereby incorporated by reference.

The articles “a” and “an” are used herein to refer to one or to more than one (i.e. at least one) of the grammatical object of the article. By way of example, “an element” means at least one element and can include more than one element.

As used herein, the term “noninvasive” refers to a procedure which inflicts minimal harm to a subject. In the case of clinical applications, a noninvasive sampling procedure can be performed quickly; e.g., in a walk-in setting, typically without anaesthesia and/or without surgical implements or suturing. Examples of noninvasive samples include blood, serum, saliva, urine, buccal swabs, throat cultures, stool samples and cervical smears. Noninvasive diagnostic analyses include x-rays, magnetic resonance imaging.

The term “immunosuppressive agent”, as used herein, refers to compounds which can inhibit an immune response.

A “marker” or “biomarker” is a gene whose altered level of expression in a tissue or cell from its expression level in untreated tissue or cell is associated with an affected or altered state of immunity, including disease states, such as immunosuppression, or lymphoproliferative disorder, or protective immunity. A “marker nucleic acid” is a nucleic acid (e.g., mRNA, cDNA) encoded by or corresponding to a marker or biomarker of the invention. Such marker nucleic acids include DNA (e.g., cDNA) comprising the entire or a partial sequence of any of the biomarkers or the complement of such a sequence. The marker nucleic acids also include RNA comprising the entire or a partial sequence of any biomarkers or the complement of such a sequence, wherein all thymidine residues are replaced with uridine residues, sense and anti-sense strands of genomic DNA (i.e. including any introns occurring therein), RNA generated by transcription of genomic DNA (i.e. prior to splicing), RNA generated by splicing of RNA transcribed from genomic DNA, and proteins generated by translation of spliced RNA (i.e. including proteins both before and after cleavage of normally cleaved regions such as transmembrane signal sequences). As used herein, a “marker” may also include a cDNA made by reverse transcription of an RNA generated by transcription of genomic DNA (including spliced RNA). A “marker protein” is a protein encoded by or corresponding to a marker of the invention. The terms “protein” and “polypeptide’ are used interchangeably.

The term “probe” refers to any molecule which is capable of selectively binding to a specifically intended target molecule, for example a marker of the invention. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

The “normal” level of expression of a marker is the level of expression of the marker in cells in a similar environment or response situation, in a subject with unaltered immunity, including one not afflicted with a disease state, such as immunosuppression, autoimmunity or a lymphoproliferative disorder or an unvaccinated subject, or a patient prior to treatment with the test agent. A normal level of expression of a marker may also refer to the level of expression of a “control sample”, (e.g., sample from a healthy subject not having the marker-associated disease state), preferably, the average expression level of the marker in several control samples. A control sample may be comprised of a control database. Alternatively, a “normal” level of expression of a marker is the level of expression of the marker in non-disease cells in a similar environment or response situation from the patient.

“Over-expression” and “under-expression” of a marker refer to expression of the marker of a patient at a greater or lesser level, respectively, than normal level of expression of the marker (e.g. more than one and a half-fold, at least two-fold, at least three-fold, greater or lesser level etc.) in a test sample that is greater than the standard error of the assay employed to assess expression. A “significant” expression level may refer to level which either meets or is above or below a pre-determined score for a biomarker set as determined by methods provided herein.

A “transcribed polynucleotide” or “nucleotide transcript” or “transcript” refers to a polynucleotide (e.g. an mRNA, hnRNA, a cDNA, or an analog of such RNA or cDNA) which is complementary to or homologous with all or a portion of a mature mRNA made by transcription of a marker of the invention and normal post-transcriptional processing (e.g. splicing), if any, of the RNA transcript, and reverse transcription of the RNA transcript.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

As used herein, the term “agent” is defined broadly as anything that a patient or isolated cells may be exposed to in a therapeutic or in vitro protocol. In the context of the present invention, such agents include, but are not limited to, immumodulatory agents, as well as chemotherapeutic agents as described in further detail herein.

Unless otherwise specified herewithin, the terms “antibody” and “antibodies” broadly encompass naturally-occurring forms of antibodies (e.g., IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

A kit is any article of manufacture (e.g. a package or container) comprising at least one reagent, e.g. a probe, for specifically detecting a marker or marker set of the invention. The article of manufacture may be promoted, distributed, or sold as a unit for performing the methods of the present invention. The reagents included in such a kit comprise nucleic acid probes/primers and/or antibodies for use in biomarker expression. In addition, the kits of the present invention may preferably contain instructions which describe a suitable detection assay. Such kits can be conveniently used, e.g., in clinical settings, to diagnose and evaluate patients at risk of exhibiting symptoms of a disease state, such as immunosuppression or a lymphoproliferative disorder, in particular patients exhibiting the possible presence of germinal center atrophy after treatment with an immunomodulator, including, e.g., patients being treated for chronic disease.

Described herein is the assessment of immunocompetence through measurement of the activity of germinal centers. Also described are assessing such capacity by noninvasive, convenient or low-cost means, for example, in blood samples. Typical methods to determine germinal center activity employ tissue sections (which typically are prepared from a biopsy of a tissue, an invasive procedure involving collecting tissue for morphological analysis; not practiced for many autoimmune indications), or cumbersome and inconvenient vaccination tests, e.g., T cell-dependent antibody responses (TDAR), or antigen exposure tests, e.g. a test for delayed-type hypersensitivity (e.g., the tuberculin test). The invention provides methods for determining, assessing, advising or providing an appropriate therapy regimen for treating a chronic disease in a patient. Monitoring a treatment using the kits and methods disclosed herein can identify the potential for adverse events and allow their prevention, and thus a savings in morbidity, mortality and treatment costs through adjustment in the therapeutic regimen, cessation of therapy or use of alternative therapy. The invention provides compositions, kits and methods for measuring the activity of germinal centers and could be widely utilized as a non-invasive means of assessing germinal center activity and the immunocompetence of the adaptive immune system.

The compositions, kits, and methods of the invention have the following uses, among others:
a) assessing the status of adaptive immunity in a human patient;
b) assessing the degree of immunosuppression in a human patient;
c) assessing the degree of immunoactivation in a patient;
d) assessing the immumodulatory potential of a test agent;
e) determining whether adaptive immunity has recovered after removal of immunomodulatory agent;
f) predicting the clinical outcome of a patient in disorders associated with changes in immunoglobulin isotype class switching;
g) assessing whether a patient is afflicted with a disease that perturbs immunoglobulin isotype class switching, such as the immunodefficiencies with hyper-IgM: HIGM1, HIGM 2, HIGM 3, HIGM 4 & HIGM 5 (e.g., Common variable immunodeficiency (CVID) and Immunoglobulin-A deficiency);
h) assessing the histological type of neoplasm;
i) assessing effectiveness of treatment against Diffuse Large B-Cell Lymphoma;
j) assessing the presence of differentiating or mature B cells;
k) predicting whether a patient is at risk for contracting an infection;
l) predicting whether a patient is at risk for contracting lymphoma or leukemia;
m) assessing the efficacy of a immunomodulatory therapy in a patient;
n) monitoring the efficacy of a vaccination;
o) selecting a composition or therapy for treating chronic disease in a patient;
p) assessing the immunomodulatory potential of a test compound;
q) preventing the onset of opportunistic infection in a patient; and
r) assessing the immunomodulatory potential of an environmental toxin.

Immature lymphocytes, e.g., immature B cells from blood, typically enter a secondary lymphoid organ, e.g., spleen, lymph node or Peyer's patch, (and sometimes additional sites of chronic inflammation, e.g., synovium if there is joint inflammation or mucosa if there is respiratory inflammation) where, upon direction by T cells, which had been stimulated by antigen and are secreting instructional cytokines, they undergo clonal expansion, differentiation, and affinity maturation- and commence production of antibodies.

Clonal expansion is the proliferation of antigen-activated B cells and is a mechanism for increasing the magnitude of an antibody response. It can be monitored by measuring markers of proliferating cells and/or the number of B cells within samples. The expression of CD19 and CD20 is unique to B lymphocytes and their expression does not appear to be regulated by IKKβ and NF-κβ activity. Therefore, biomarkers for splenic germinal center atrophy can be CD19 or CD20, whose levels in peripheral blood can be measured to determine the level of B lymphocytes.

Immunoglobulin isotype class switching (ICS) is a differentiation activity whereby IgM, the isotype of the primary antibody response (i.e., what a B cell would produce without any differentiation instructions from a T cell), is switched to immunoglobulins type G, A or E (IgG, IgA, or IgE, respectively) during the secondary antibody response. Switching the isotype of an antibody response brings additional, alternative effector functions of the immune system to bear in combating a pathogen. Immunoglobulin ICS therefore can be monitored by measuring IgG, IgA, and IgE levels (Snapper et al. (1997) Immunity 6:217-23, Chaudhuri and Alt (2004) Nat. Rev. Immunol. 4:541-52, 655). Immunoglobulin ICS can be monitored by measuring IgG, IgA, and IgE levels in peripheral blood of laboratory animals housed in aseptic environments. These techniques are not effective in animals or subjects not housed in aseptic environments, as their immune systems have been stimulated previously and consequently harbor large reservoirs of Ig, precluding detection of the relatively small changes related specifically to germinal center activity. Due to the large pre-existing pool of immunoglobulins (Igs) that exhibit relatively long half-lives, it can be important to wait for a decrease in the level of pre-existing Igs before monitoring changes in production de novo. The half-life of serum IgG, for example, is 27 days.

Germinal center activity preferably is measured by quantifying evidence of differentiation and/or proliferation of B cells. In certain aspects, determining or confirming a value for a parameter related to a patient's germinal center activity comprises detection of mRNA. Such detection can be carried out by any relevant method, including e.g., PCR, northern, nucleotide array detection, in vivo imaging using probes capable of detection of the appropriate nucleic acid. In other aspects, determining a value for a parameter related to the expression of a biomarker in a sample comprises detection of protein. Such detection can be carried out using any relevant method for protein detection, including e.g., ELISA, western blot, immunoassay, protein array detection, in vivo imaging using probes capable of detection of the appropriate peptide. A standard way of measuring immunocompetence is a vaccination study, also referred to as the T cell-dependent antibody response (TDAR) in nonclinical investigations. In the TDAR test, the ability of the subject to mount a response to a new antigen is measured. It is performed by immunizing the subject with a foreign substance, e.g., keyhole limpet hemocyanin, and determining the amount of recall upon reintroduction. Vaccination studies in clinical trials or during routine therapeutic treatment are laborious, inconvenient, costly and can present risk of adverse reactions in subjects. Another way of testing is to identify the composition of B cells in peripheral blood by Flow Cytometry. However, Flow Cytometry cannot be utilized to assay samples at some clinical sites, worldwide (i.e., Ph III trials, ambulatory clinics), as it uses expensive machinery and requires trained operators.

Assays that monitor transcription, e.g., measure Ig transcripts, are preferable for early detection of atrophy because a) transcription is terminated prior to translation, b) Ig transcripts are less stable than proteins (they have shorter half-lives), and c) several meaningful transcripts, such as switch transcripts, are not translated into proteins. Switch transcripts are intermediary products unique to ICS that present a potential biomarker for splenic germinal center atrophy (Snapper et al. (1997) Immunity 6:217-23, Chaudhuri and Alt (2004) Nat. Rev. Immunol. 4:541-52; erratum pg. 655). Germline switch transcripts, e.g., germline transcript mu (GLT-μ) are generated immediately prior to switch recombination, when chromatin decondenses around an Ig gene locus, the substrate for the deoxyribonucleic acid (DNA) recombination reaction that characterizes ICS (Snapper et al., supra, Chaudhuri and Alt, supra). Circle transcripts, e.g., those containing gamma 1 and 2 (CT-γ1 and CT-γ2) are another type of switch transcript and are produced later in the ICS process. They are transcripts from the excised genomic DNA that are essentially the by-products of DNA recombination during ICS (Snapper et al., supra, Chaudhuri and Alt supra). Measuring levels of these transcripts permits identification of which stage of DNA recombination is occurring. Additional genes are required for DNA recombination during human ICS, such as activation-induced cytidine deaminase (AID, AICDA; Revy et al. (2000) Cell 102:565-75, Imai et al. (2003) Nature Immunol. 4:1023-28), uracil-DNA glycosylase (UNG, Imai et al., supra, severe-combined immunodeficiency (SOD) gene product (Rolink et al. (1996) Immunity 5:319-30), Ku heterodimer (ku70/ku86, Manis et al. (1998) J. Exp. Med. 187:2081-9), or ku80 (Casellas et al. (1998) EMBO J. 17:2404-11) expression or nuclear localization or activity of DNA-dependent serine/threonine protein kinase (DNA-PK; p350) (Snapper et al., supra; Zelazowski et al. (1997) J. Immunol. 159:2559-62) or recombination activating gene 1 (RAG1, Girschick et al. (2001) J. Immunol. 166:377-86).

Homeostasis entails the migration of B lymphocytes that participated in a germinal center reaction from secondary lymphoid organs to other organs (e.g., the bone marrow) via the vasculature. Expression of AID is exclusively restricted to B lymphocytes that have recently participated in a germinal center reaction. Mutations in the AID gene prevent ICS and are characterized by the presence of germline, but not circle, transcripts, by hyperplastic (i.e., giant) germinal centers, and by profound immunodeficiency (e.g., hyper-immunoglobulin M types 2 and 5 (HIGM2 and HIGM5)). Both humans and mice express AID and germline switch transcripts indicative of early ICS activity, but not the circle transcripts resulting from DNA recombination indicative of late ICS activity (Revy et al., supra, Imai et al., supra). Measuring levels of switch transcripts and AICDA expression therefore provides information on what stage of ICS is affected, illustrating their potential utility in monitoring atrophy of germinal centers and immunosuppression in response to a test agent. Human immunodeficiences with hyper-immuglobulin (Ig) M, types 1 and 3 (HIGM1 and HIGM3, which abolish NF-κB signaling in B cells, also are characterized by hypoplastic germinal centers, immune deficiency and recurrent bacterial infections (Ramesh et al. (1999) Primary Immunodeficiency Diseases: A Molecular and Genetic Approach, Oxford University Press, New York, pp. 233-49, Castigli et al. (1994) Proc. Natl. Acad. Sci. USA 91:12135-9, Ferrari et al. (2001) Proc. Natl. Acad. Sci. USA 98:12614-9). Expression of AID is unique to B lymphocytes, whereas UNG is ubiquitously expressed. Moreover, these switch transcripts exist in peripheral blood because homeostasis involves migration of memory B cells and plasmablasts from secondary lymphoid organs to the bone marrow via the vasculature (Kunkel and Butcher (2003) Nat. Rev. Immunol. 3:822-9).

Alternatively, affinity maturation could be used as a biomarker for splenic germinal center atrophy by measuring somatic mutation of IgS. However, this is a labor-intensive and relatively expensive methodology.

Exemplary biomarkers to measure include markers of germline activation, including germline switch transcripts containing immunoglobulin chains, e.g., mu, delta, gamma, alpha or epsilon heavy chains (e.g., germline transcript mu (GLT-μ) e.g., SEQ ID NO:1, in the region of bases 961381 to 967501 of GenBank Accession No. NG_001019 (Jan. 10, 2006 version), or other sequences at the immunoglobulin heavy (IGH) locus on chromosome 14); circle switch transcripts, containing immunoglobulin chains, e.g., mu, delta, gamma, alpha or epsilon heavy chains (e.g., those containing gamma 1 and 2 (CT-γ1 and CT-γ2, e.g., SEQ ID NO:2, in the region of bases 967201 to 1048261 of NG_001019 (Jan. 10, 2006 version)) or other sequences at the immunoglobulin heavy (IGH) locus on chromosome 14); circular DNA fragments containing mu, delta, gamma, alpha or epsilon heavy chains; and/or DNA recombination effectors (e.g., activation-induced cytidine deaminase, AID or AIDCA, GenBank Accession No. NM_020661, SEQ ID NOs:3,4; UNG, GenBank Accession No. NM_080911, SEQ ID NOs:5,6; Ku70, GenBank Accession No. NM_001469, SEQ ID NOs:7,8; Ku80, GenBank Accession No. NM_021141, SEQ ID NOs:9,10; or RAG1, NM_000448, SEQ ID NOs:11,12) or light chains (e.g., immunoglobulin light chain kappa (IgL-κ) GenBank Accession No. BC070336, SEQ ID NOs:13,14; BC012159, SEQ ID NOs:15,16). Preferable biomarkers have expression limited to germinal center B cells. Preferred biomarkers to detect as disclosed herein include GLT-μ, AICDA (AID), CT-γ1&2, IGHG1, e.g., in the region of bases 1080134-1081837 of NG_001019, e.g., NC_000014, SEQ ID NO:20; IGHG2; IGHG3; IGHG4; IGHA1, e.g., in the region of bases 1114540-1116066 of GenBank Accession No. NG_001019, SEQ ID NO:21; IGHA2 and/or IGHE. (The Entrez Gene database (National Center for Biotechnology Information, Bethesda, Md.), as well as the supporting information provided in NG_001019, defines the regions for each biomarker not described herein by SEQ ID NO. One of skill in the art can review the database information and readily obtain the regions for the other biomarkers, e.g., IGHG2, IGHG3, IGHG4, IGHA2, IGHE.) Most preferred biomarkers include GLT-μ, AICDA (AID), CT-γ1&2, IGHG1 and/or IGHA2.

The choice of biomarker can be adjusted according to the pathway of action of the immumodulator. For example, activation of the tumor necrosis factor-alpha (TNF-α) and NF-κB pathways results in regulation of expression of genes which can be used as markers in this method. Levels of TNF-α and Interleukin-1 Beta (IL-13) could be measured, since their expression is regulated by IKKβ and NF-κB and, as a positive control for activity of such an immunomodulator, represent a pharmacodynamic (mechanistic) marker for its activity. However, these transcripts have a ubiquitous expression pattern, meaning they are expressed in many types of blood cells. Expression of IgL-x, which also is regulated by NF-κB, also can be measured as a potential mechanistic marker for activity of the immunomodulators which affect tumor necrosis factor-alpha (TNF-α) or NF-κB pathways. However, unlike TNF-α AND IL-1β, it is target cell-specific, in that it is exclusively expressed by B lymphocytes, so measuring IGL-κ levels can allow monitoring of activity of such an immunomodulator specifically in the B cell compartment. Accordingly, if the immunomodulator has a mechanism of action that affects the TNFα or the NF-κB pathway, a preferred biomarker is IGL-κ.

The term “biological sample” is intended to include tissues, cells, biological fluids and isolates thereof, isolated from a subject, as well as tissues, cells and fluids present within a subject. Based on general histological information on the frequency of various types of cells in the blood, post-germinal center B cells (e.g., plasmablasts), as a small subset of B cells, are a very low percentage of the cell population in whole blood. Surprisingly, transcripts representative of ICS are detectable in peripheral blood samples. Thus, preferable noninvasive samples, e.g., for in vitro measurement of adaptive immunity, include peripheral blood samples. Accordingly, cells within peripheral blood can be tested for ICS. Blood collection containers preferably comprise an anti-coagulant, e.g., heparin or ethylene-diaminetetraacetic acid (EDTA), sodium citrate or citrate solutions with additives to preserve blood integrity, such as dextrose or albumin or buffers, e.g., phosphate. If the amount of biomarker is being measured by measuring the level of its RNA in the sample, an RNA stabilizer, e.g., an agent that inhibits RNAse, can be added to the sample. If the amount of biomarker is being measured by measuring the level of its protein in the sample, protein stabilizer, e.g., an agent that inhibits proteases, can be added to the sample. An example of a blood collection container is PAXGENE® tubes (PREANALYTIX, Valencia, Calif.), useful for RNA stabilization upon blood collection. Peripheral blood samples can be modified, e.g., fractionated, sorted or concentrated (e.g., to result in samples enriched with antibody-producing B cells). Examples of modified samples include plasmablasts, which can be collected by e.g., negative selection, e.g., separation of white blood cells from red blood cells (e.g., differential centrifugation through a dense sugar or polymer solution (e.g., FICOLL® solution (Amersham Biosciences division of GE healthcare, Piscataway, N.J.) or HISTOPAQUE®-1077 solution, Sigma-Aldrich Biotechnology LP and Sigma-Aldrich Co., St. Louis, Mo.)) and/or positive selection by binding B cells to a selection agent (e.g., a reagent which binds to a B cell marker, such as CD19, CD38, CD138, or CD30, for direct isolation (e.g., the application of a magnetic field to solutions of cells comprising magnetic beads (e.g., from Miltenyi Biotec, Auburn, Calif.) which bind to the B cell markers) or fluorescent-activated cell sorting). Alternatively, a B cell line, e.g., B-cell lymphoma (e.g., BC-3) can be assayed. A skilled artisan readily can select and obtain the appropriate cells (e.g., from American Type Culture Collection (ATCC®), Manassas, Va.) that are used in the present method. A sample modified to select for B cells can be useful to measure markers, e.g., Ku70, RAG1, etc., whose expression is a hallmark of ICS, but is not limited to germinal center B cells. If the compositions or methods are being used to predict capacity for adaptive immunity in a patient or monitor the effectiveness of a therapeutic protocol, then a tissue or blood sample from the patient being treated is a preferred source.

The sample, e.g., blood or modified blood, can be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample.

In a particular embodiment, the level of mRNA corresponding to the marker can be determined both by in situ and by in vitro formats in a biological sample using methods known in the art. Many expression detection methods use isolated RNA. For in vitro methods, any RNA isolation technique that does not select against the isolation of mRNA can be utilized for the purification of RNA from tumor cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155). RNA can be isolated using standard procedures (see e.g., Chomczynski and Sacchi (1987) Anal. Biochem. 162:156-159), solutions (e.g., trizol, TRI REAGENT® (Molecular Research Center, Inc., Cincinnati, Ohio; see U.S. Pat. No. 5,346,994) or kits (e.g., a QIAGEN® Group RNEASY® isolation kit (Valencia, Calif.) or LEUKOLOCK™ Total RNA Isolation System, Ambion division of Applied Biosystems, Austin, Tex.).

Additional steps may be employed to remove DNA. Cell lysis can be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et al. (1979) Biochemistry 18:5294-99). Poly(A)+RNA is selected by selection with oligo-dT cellulose (see Sambrook et al. (1989) Molecular Cloning—A Laboratory Manual (2nd ed.), Cold Spring Harbor Laboratory, Cold Spring Harbor, N.Y.). Alternatively, separation of RNA from DNA can be accomplished by organic extraction, for example, with hot phenol or phenol/chloroform/isoamyl alcohol. If desired, RNAse inhibitors may be added to the lysis buffer. Likewise, for certain cell types, it may be desirable to add a protein denaturation/digestion step to the protocol. For many applications, it is desirable to preferentially enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or SEPHADEX® medium (see Ausubel et al. (1994) Current Protocols In Molecular Biology, vol. 2, Current Protocols Publishing, New York). Once bound, poly(A)+mRNA is eluted from the affinity column using 2 mM EDTA/0.1% SDS.

The sample of RNA can comprise a plurality of different RNA molecules, each different RNA molecule having a different nucleotide sequence. In a specific embodiment, the RNA molecules in the RNA sample comprise at least 100 different nucleotide sequences. More preferably, the RNA molecules of the RNA sample comprise RNA molecules corresponding to each of the marker genes.

The level of germinal center activity, e.g., the extent of isotype class switching or clonal expansion of B lymphocytes, can be measured using any suitable assay, for example, directly or indirectly. Expression of a marker of the invention may be assessed by any of a wide variety of well known methods for detecting expression of a transcribed nucleic acid and/or translated protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods. These methods, include gene array/chip technology, RT-PCR, in situ hybridization, immunohistochemistry, immunoblotting, FISH (fluorescence in situ hybridization), FACS analyses, northern blot, southern blot or cytogenetic analyses. The detection methods of the invention can thus be used to detect RNA, mRNA, protein, cDNA, or genomic DNA, for example, in a biological sample in vitro as well as in vivo. Furthermore, in vivo techniques for detection of a polypeptide or nucleic acid corresponding to a marker of the invention include introducing into a subject a labeled probe to detect the biomarker, e.g., a nucleic acid complementary to the transcript of a biomarker or a labeled antibody, Fc receptor or antigen directed against the polypeptide, e.g., immunoglobulin or DNA recombination effector. For example, the antibody can be labeled with a radioactive marker whose presence and location in a subject can be detected by standard imaging techniques. These assays can be conducted in a variety of ways. A skilled artisan can select from these or other appropriate and available methods based on the nature of the marker(s), tissue sample and isotype in question. Some methods are described in more detail in later sections. Different methods or combinations of methods could be appropriate in different cases or, for instance in different chronic diseases or patient populations.

An exemplary method for detecting the presence or absence of nucleic acid corresponding to a biomarker of the invention in a biological sample involves obtaining a biological sample (e.g., a blood sample) from a test subject and contacting the biological sample with a compound or an agent capable of detecting the nucleic acid (e.g., RNA, mRNA, genomic DNA, or cDNA). For example, in vitro techniques for detection of mRNA include PCR, northern hybridizations, in situ hybridizations, nucleotide array detection, and TAQMAN® gene expression assays (Applied Biosystems, Foster City, Calif.), preferably under GLP approved laboratory conditions. In vitro techniques for detection of genomic DNA include Southern hybridizations.

In one embodiment, expression of a marker is assessed by preparing mRNA/cDNA (i.e., a transcribed polynucleotide) from cells in a patient sample, and by hybridizing the mRNA/cDNA with a reference polynucleotide which is a complement of a marker nucleic acid, or a fragment thereof. cDNA can, optionally, be amplified using any of a variety of polymerase chain reaction methods prior to hybridization with the reference polynucleotide; preferably, it is not amplified. Expression of one or more markers likewise can be detected using quantitative PCR to assess the level of expression of the marker(s). Alternatively, any of the many known methods of detecting mutations or variants (e.g. single nucleotide polymorphisms, deletions, etc.) of a marker of the invention may be used to detect occurrence of a marker in a patient. For example, measurement of mutated forms of AICDA (AID) can indicate the level of immunocompetence (Revy et al. (2000) Cell 102:565-75).

In vitro techniques for detection of a polypeptide corresponding to a marker of the invention include enzyme linked immunosorbent assays (ELISAs), Western blots, protein array, immunoprecipitations and immunofluorescence. In such examples, expression of a marker is assessed using an antibody (e.g., a radio-labeled, chromophore-labeled, fluorophore-labeled, or enzyme-labeled antibody), an antibody derivative (e.g., an antibody conjugated with a substrate or with the protein or ligand of a protein-ligand pair (e.g., biotin-streptavidin)), or an antibody fragment (e.g., a single-chain antibody, an isolated antibody hypervariable domain, etc.) which binds specifically with a marker protein or fragment thereof, including a marker protein which has undergone all or a portion of its normal post-translational modification.

An example of direct measurement is quantification of transcripts. As used herein, the level or amount of expression refers to the absolute level of expression of an mRNA encoded by the marker or the absolute level of expression of the protein encoded by the marker. As an alternative to making determinations based on the absolute expression level of selected markers, determinations may be based on normalized expression levels. Expression levels are normalized by correcting the absolute expression level of a biomarker upon comparing its expression to the expression of a control marker that is not a biomarker, e.g., in a housekeeping role that is constitutively expressed. Suitable markers for normalization also include housekeeping genes, such as the actin gene or beta-2 microglobulin. Reference biomarkers for data normalization purposes include markers which are ubiquitously expressed and/or whose expression is not regulated by immunomodulators. Preferred reference markers include 18S ribosomal RNA (18S, GenBank Accession No. X03205, SEQ ID NO:17), and transcripts of beta-2 microglobulin (B2M, GenBank Accession No. NM_004048, SEQ ID NOs:18,19). Constitutively expressed genes are known in the art and can be identified and selected according to the relevant tissue and/or situation of the patient and the analysis methods. Such normalization allows one to compare the expression level in one sample, to another sample, e.g., between samples from different times or different subjects. Further, the expression level can be provided as a relative expression level. To determine a relative expression level of a marker or marker set, the level of expression of the biomarker or biomarker set is determined for at least 1, preferably 2, 3, 4, 5, or more samples, e.g., 7, 10, 15, 20 or 50 or more samples in order to establish a baseline, prior to the determination of the expression level for the sample in question. To establish a baseline measurement, the mean expression level of each of the biomarkers or biomarker sets assayed in the larger number of samples is determined and this is used as a baseline expression level for the biomarkers or biomarker sets in question. The expression level of the biomarker or biomarker set determined for the test sample (absolute level of expression) is then divided by the baseline expression value obtained for that biomarker or biomarker set. This provides a relative expression level and aids in identifying extreme levels of germinal center activity.

Some markers, e.g., AICDA (AID) and IGL-κ, are expressed as mRNAs (e.g., are poly-adenylated) and ultimately translated as proteins and some markers, e.g., GLT-μ and the CT's, are not. Thus, the detection methods and measurement of expression levels are selected accordingly. Choices including cDNA amplification methods and protein detection methods can be used for translated biomarkers; nucleic acid measurement and amplification methods are used for biomarkers which are not translated. Primers and nucleic acid probes can be optimized for the particular transcripts. Commercial assays for transcripts used in ICS are available for use with quantitative RT-PCR systems, e.g., Assay-On-Demand formats from Applied Biosystems, Inc., Foster City, Calif. Modifications of the system can enable high throughput analyses of the samples, e.g., the TAQMAN® low density array upgrade (Applied Biosystems, Foster City, Calif.).

Preferred primers or nucleic acid probes comprise a nucleotide sequence complementary to a specific allelic variant of a biomarker polymorphic region and of sufficient length to selectively hybridize with a biomarker gene. In a preferred embodiment, the primer or nucleic acid probe, e.g., a substantially purified oligonucleotide, comprises a region having a nucleotide sequence which hybridizes under stringent conditions to about 6, 8, 10, or 12, preferably 15, 20, 25, 30, 40, 50, 60, 75, 100 or more consecutive nucleotides of a biomarker gene. In an even more preferred embodiment, the primer or nucleic acid probe is capable of hybridizing to a biomarker nucleotide sequence and comprises a nucleotide sequence of any sequence set forth in any of SEQ ID NOs:22-57, or a complement thereof. For example, a primer or nucleic acid probe comprising a nucleotide sequence of at least about 15 consecutive nucleotides, at least about 25 nucleotides or having from about 15 to about 20 nucleotides set forth in any of SEQ ID NOs:22-57 or a complement thereof are provided by the invention. Primers or nucleic acid probes having a sequence of more than about 25 nucleotides are also within the scope of the invention. In another embodiment, a primer or nucleic acid probe can have a sequence at least 70%, preferably 75%, 80% or 85%, more preferably, 90%, 95% or 97% identical to the nucleotide sequence of any sequence set forth in any of SEQ ID NOs:22-57, or a complement thereof. Nucleic acid analogs can be used as binding sites for hybridization. An example of a suitable nucleic acid analogue is peptide nucleic acid (see, e.g., Egholm et al., Nature 363:566 568 (1993); U.S. Pat. No. 5,539,083). Primers or nucleic acid probes are preferably selected using an algorithm that takes into account binding energies, base composition, sequence complexity, cross-hybridization binding energies, and secondary structure (see Friend et al., International Patent Publication WO 01/05935, published Jan. 25, 2001; Hughes et al., Nat. Biotech. 19:342-7 (2001). Preferred primers or nucleic acid probes of the invention are primers that bind sequences which are unique for each transcript and can be used in PCR for amplifying and detecting only that particular transcript. One of skill in the art can design primers and nucleic acid probes for the biomarkers disclosed herein or related biomarkers with similar characteristics, e.g., biomarkers having a role in ICS or germinal center activity, using the skill in the art, e.g., adjusting the potential for primer or nucleic acid probe binding to standard sequences, mutants or allelic variants by manipulating degeneracy or GC content in the primer or nucleic acid probe. Computer programs that are well known in the art are useful in the design of primers with the required specificity and optimal amplification properties, such as Oligo version 5.0 (National Biosciences, Plymouth, Minn.). While perfectly complementary nucleic acid probes and primers are preferred for detecting the biomarkers described herein and polymorphisms or alleles thereof, departures from complete complementarity are contemplated where such departures do not prevent the molecule from specifically hybridizing to the target region. For example, an oligonucleotide primer may have a non-complementary fragment at its 5′ end, with the remainder of the primer being complementary to the target region. Alternatively, non-complementary nucleotides may be interspersed into the nucleic acid probe or primer as long as the resulting probe or primer is still capable of specifically hybridizing to the target region.

Preferred sequences to detect for the measurement of GLT-μ (e.g., to detect SEQ ID NO:1) comprise a fragment of at least 10, 15, 20, 25, 30, 35, 50, 75, or 100 or more nucleotides of the following sequence or complement thereof: SEQ ID NO:58 for 5′ primer and/or SEQ ID NO:59, for the probe and 3′ primer. Examples of 5′ primers or complement thereof to use when detecting, amplifying and/or measuring GLT-μ are SEQ ID NOs:53, 55 or 34 (GLT1, GLT2 and GLT3, respectively). An example of a probe or complement thereof to use when detecting, amplifying and/or measuring GLT-μ is SEQ ID NO: 36. Examples of 3′ primers, or reverse complement thereof to use when detecting, amplifying and/or measuring GLT-μ are SEQ ID NOs:35 or 54. Nucleic acid sequences comprising the above GLT-μ primers and probe (SEQ ID NOs:53, 55, 34, 36, 35 or 54), or complements thereof, can be used separately or together as a set for quantitative RT-PCR.

Preferred sequences to detect for the measurement of CT-γ1&2 (e.g., to detect SEQ ID NO:2) comprise a fragment of at least 10, 15, 20, 25, 30, 35, 50, 75, or 100 or more nucleotides of the following sequences or complements thereof: SEQ ID NO:60 for 5′ primer and/or SEQ ID NO:59 for the probe and 3′ primer. Examples of 5′ primer or complement thereof to use when detecting, amplifying and/or measuring CT-γ1&2 are SEQ ID NOs:37, 56, or 57 (CT1, CT2 and CT3, respectively). An example of a probe or complement thereof to use when detecting, amplifying and/or measuring CT-γ1&2 is SEQ ID NO:36. Examples of 3′ primers, or reverse complement thereof to use when detecting, amplifying and/or measuring CT-γ1&2 are SEQ ID NOs:35 or 54. Nucleic acid sequences comprising the above CT-γ1&2 primers and probe (SEQ ID NOs:37, 56, 57, 36, 35, or 54), or complements thereof, can be used separately or together as a set for quantitative RT-PCR.

An indication of germinal center activity can be assessed by studying the level of 1 biomarker, 2 biomarkers, 3 biomarkers, 4 biomarkers, 5 biomarkers, 6 biomarkers, biomarkers, 8 biomarkers, 9 biomarkers, 10 biomarkers, or more, e.g., 15, 20 or 25 biomarkers. Biomarkers can be studied in combination with another measure of immune competence, e.g., histological markers (i.e., hypo- or hyperplasia of secondary lymphoid organs), frequency of subsets of leukocytes, level of cytokine, T cell-dependent antibody responses (TDAR).

Statistical methods can assist in the determination of germinal center activity upon measurement of the level of expression of biomarkers, e.g., measurement of transcripts. The level of one transcript can be measured at multiple timepoints, e.g., before treatment, during treatment, after treatment with an agent, e.g., an immunomodulator. To determine the progression of change in expression of a marker from a baseline, e.g., over time, the expression results can be analyzed by a repeated measures linear regression model (Littell, Miliken, Stroup, Wolfinger, Schabenberger (2006) SAS for Mixed Models, 2^{nd} edition. SAS Institute, Inc., Cary, N.C.)):

Yᵢⱼₖ−Yᵢⱼ₀=Yᵢⱼ₀+treatmentᵢ+dayₖ+(treatment*day)ᵢₖ+εᵢⱼₖ Equation 1

where Yᵢⱼₖ is the log₂ transformed expression (normalized to the housekeeping genes) on the k^{th} day of the j^{th} animal in the i^{th} treatment, Yᵢⱼ₀ is the defined baseline log₂ transformed expression (normalized to the housekeeping genes) of the j^{th} animal in the i^{th} treatment, dayₖ is treated as a categorical variable, and εᵢⱼₖ is the residual error term. A covariance matrix (e.g., first-order autoregressive, compound symmetry, spatial power law) can be specified to model the repeated measurements on each animal over time. Furthermore, each treatment time point can be compared back to the same time point in the vehicle group to test whether the treatment value was significantly different from vehicle.

A number of other methods can be used to analyze the data. For instance, the relative expression values could be analyzed instead of the cycle number. These values could be examined as either a fold change or as an absolute difference from baseline. Additionally, a repeated-measures analysis of variance (ANOVA) could be used if the variances are equal across all groups and time points. The observed change from baseline at the last (or other) time point could be analyzed using a paired t-test, or a Wilcoxon signed rank test if the data is not normally distributed, to compare whether a treated group was significantly different from the vehicle group.

A difference in expression from one timepoint to the next can indicate a change in germinal center activity. A baseline level can be determined by measuring expression at 1, 2, 3, 4, or more times prior to treatment, e.g., at time zero, one day, three days, one week and/or two weeks or more before treatment. Alternatively, a baseline level can be determined from a number of subjects, e.g., normal subjects or patients with the same health status or disorder, who do not undergo or have not yet undergone the treatment, as discussed above. Alternatively, one can use expression values deposited with the Gene Expression Omnibus (GEO) program at the National Center for Biotechnology Information (NCBI, Bethesda, Md.). To test the effect of the immunomodulator on immunocompetence, the expression of the biomarker can be measured at any time or multiple times after some treatment, e.g., after I day, 2 days, 3 days, 5 days, 1 week, 2 weeks, 3 weeks, 4 weeks, 1 month, 2 months, 3 months and/or 6 or more months of treatment. For example, the level of expression of a biomarker can be measured once after some treatment, or at multiple intervals, e.g., 1-week, 2-week, 4-week or 2-month, 3-month or longer intervals during treatment. Conversely, to determine whether there has been recovery or restoration of normal immunocompetence after stopping the administration of an immunomodulator, the expression of the biomarker can be measured at any time or multiple times after, e.g., 1 day, 2 days, 3 days, 5 days, 1 week, 2 weeks, 3 weeks, 4 weeks, 1 month, 2 months, 3 months and/or 6 or more months after the last treatment. One of skill in the art would determine the timepoint or timepoints to assess the expression level of the biomarker depending on various factors, e.g., the pharmacokinetics of the immunomodulator, the treatment duration, pharmacodynamics of the immunomodulator, age of the patient, the nature of the disorder or mechanism of action of the immunomodulator. A trend in the negative direction or a decrease in the amount relative to baseline or a pre-determined standard of expression of a biomarker of immune competence indicates a decrease in germinal center activity, e.g., atrophy. A trend in the positive direction or an increase in the amount relative to the baseline or a pre-determined standard of expression of a biomarker of immune competence indicates an increase in germinal center activity, e.g., hyperplasia or clonal expansion.

Because the compositions, kits, and methods of the invention rely on detection of a difference in expression levels of one or more markers of the invention, it is preferable that difference in the level of expression of the marker is significantly greater than the minimum detection limit of the method used to assess expression in at least one of baseline levels and treated levels. Preferably, changes in germinal center activity are measured by 2-, 2.5-, 3-, 3.5-, 4-, 4.5-, 5-, 7-, 10-fold, or more differences of expression of the biomarker. For example, 2-, 2.5-, 3-, 3.5-, 4-, 4.5-, 5-, 7-, 10-fold or more higher expression would indicate higher germinal center activity; 2- (i.e., one-half), 2.5-, 3-, 3.5-, 4-, 4.5-, 5-, 7-, 10- (i.e., one tenth) fold or less lower expression would indicate lower germinal center activity. Any marker or combination of markers of the invention, as well as any known markers in combination with the markers of the invention, may be used in the compositions, kits, and methods of the present invention. In general, it is preferable to use markers for which the difference between the level of expression of the marker in cells from atrophied or hyperplastic germinal centers and the level of expression of the same marker in cells from normal germinal centers is as great as possible. Although this difference can be as small as the limit of detection of the method for assessing expression of the marker, it is preferred that the difference be at least greater than the standard error of the assessment method, and preferably a difference of at least 2-, 3-, 4-, 5-, 6-, 7-, 8-, 9-, 10-, 15-, 20-, 25-, 100-, 500-, 1000-fold or greater than when the level of expression of the same marker is measured in samples comprising B cells which have not experienced a germinal center or which have not experienced the test agent. The difference can be qualified by a confidence level, e.g., p<0.05, preferably, p<0.02, more preferably p<0.01.

Measurement of more than one biomarker, e.g., a set of 2, 3, 4, 5, 6, 7, 8, 9, 10, 12, 15, 20, or 25 or more biomarkers can provide an expression profile or a trend indicative of germinal center activity. In some embodiments, the predictive marker set comprises no more than 2, 3, 4, 5, 6, 7, 8, 9, 10, 12, 15, 20, or 25 biomarkers. In some embodiments, the predictive marker set includes a plurality of genes associated with isotype class switching, clonal expansion or maturation of B cells. Analysis of germinal center activity through assessing expression of biomarkers in a set can be accompanied by a statistical method, e.g., a weighted voting analysis which accounts for variables which can affect the contribution of the expression level of a marker in the set to the class or trend of germinal center activity, e.g., the signal-to-noise ratio of the measurement or hybridization efficiency for each biomarker. A biomarker set, e.g., a set of 2, 3, 4, 5, 6, 7, 8, 9, 10, 12, 15, 20, or 25 or more biomarkers, comprises a probe or probes to detect at least one biomarker described herein, e.g., a marker indicative of ICS, (e.g., 18S ribosomal RNA and/or beta-2 microglobulin (B2M (for data normalization purposes), and GLT-μ, AICDA (AID), CT-γ1&2, Ku70, RAG1, IGHG1, IGHG2, IGHG3, IGHG4, IGHA1, IGHA2 and/or IGHE. A preferred biomarker set, e.g., a set of 2, 3, 4, 5, 6, 7, 8, 9, 10, 12, 15, 20, or 25 or more biomarkers, comprises a probe or probes to detect at least one or at least two or more preferred biomarkers, e.g., at least one or at least two of GLT-μ, AICDA (AID), CT-γ1, CT-γ2, IGHG1 and/or IGHA2. If the immunomodulator has a mechanism of action that targets TNFα or the NF-κB pathway, a preferred biomarker to include in a biomarker set is IGL-κ. Selected biomarker sets can be assembled from the biomarkers provided herein or selected from among germinal center activators, ICS expression products or ICS effectors using methods provided herein and analogous methods known in the art. A way to qualify a new marker for use in an assay of the invention is to correlate histological changes in germinal center activity with differences in expression (e.g., fold-change from baseline) of an ICS biomarker. A useful way to judge the relationship is to calculate the coefficient of determination r², after solving for r, the Pearson product moment correlation coefficient and/or preparing a least squares plot, using standard statistical methods. A preferable correlation would analyze reactivity of germinal centers (inverse of atrophy) versus the level of ICS biomarker. Preferably, a gene product would be selected as a biomarker of ICS if the result of the correlation (r², e.g., the linear slope of the data in this analysis), is at least 0.1-0.2, more preferably, at least 0.3-0.5, most preferably at least 0.6-0.8 or more, up to 1.0 (i.e., perfect positive correlation). Some biomarkers can vary with a positive correlation to germinal center activity (i.e., change expression levels in the same manner as germinal center activity levels, e.g., decrease when germinal center activity is decreased) and some biomarkers can vary with a negative correlation to germinal center activity (i.e., change expression levels in the opposite manner as germinal center activity levels, e.g., increase when germinal center activity is decreased).

Another way to qualify a new marker for use in the assay would be to assay the expression of large numbers of germinal center activators, ICS expression products or ICS effectors in a number of subjects before and after treatment with a test agent. The expression results allow identification of the markers which show large changes in a given direction after treatment relative to the pre-treatment samples. One can build a repeated-measures linear regression model to identify the genes that show statistically significant changes. To then rank these significant genes, one can calculate the area under the change from baseline vs time curve. This can result in a list of genes that would show the largest statistically significant changes. A review of the function of the genes with large changes in expression could help qualify the suitability of the gene for assessing a trend, e.g. germinal center hyperplasia or atrophy (e.g., predictive of immunosuppression), depending on the gene's role in germinal center activity or ICS. We could combine several genes together in a set by using such methods as principle component analysis, clustering methods (e.g., k-means, hierarchical), multivariate analysis of variance (MANOVA), or linear regression techniques. To use such a gene (or group of genes) as a marker, genes which show 2-, 2.5-, 3-, 3.5-, 4-, 4.5-, 5-, 7-, 10-fold, or more differences of expression from baseline would be included in the marker set. An expression profile, e.g., a composite of the expression level differences from baseline or reference of the aggregate marker set would indicate at trend, e.g., if a majority of markers show a particular result, e.g., a significant difference from baseline or reference, preferably 60%, 70%, 80%, 90%, 95% or more markers; or more markers, e.g., 10% more, 20% more, 30% more, 40% more, show a significant result in one direction than the other direction.

When the compositions, kits, and methods of the invention are used for characterizing capacity of adaptive immunity, e.g., immunosuppression or immunity in a patient, it is preferred that the biomarker or set of biomarkers of the invention is selected such that a significant result is obtained in at least about 20%, and preferably at least about 40%, 60%, or 80%, and more preferably in substantially all patients treated with the test agent. Preferably, the biomarker or set of biomarkers of the invention is selected such that a positive predictive value (PPV) of greater than about 10% is obtained for the general population (more preferably coupled with an assay specificity greater than 80%).

Thus, in one embodiment, e.g., an embodiment using a nucleic acid array, the method of determining a particular adaptive immunity-related status of an individual comprises the steps of (1) hybridizing labeled target nucleic acid probes or primers from an individual to a plate, e.g., microarray containing one of the marker sets described herein or determined according to the parameters described herein; (2) hybridizing standard or control nucleic acid probes or primers to the microarray, wherein the standard or control molecules are differentially labeled from the target molecules; and (3) determining the ratio (or difference) of transcript levels between subject and control, reference or baseline, or simply the absolute transcript levels of the individual; and (4) comparing the results from (3) to the predefined standard, profiles, templates or trends, wherein said determining can be accomplished by means of the statistic as described herein, and wherein the difference, or lack thereof, determines the individual's adaptive immunity-related status.

The present invention pertains to the field of predictive medicine in which diagnostic assays, prognostic assays, pharmacogenomics, and monitoring clinical trails can be used for prognostic (predictive) purposes to thereby treat an individual prophylactically. Accordingly, one aspect of the present invention relates to diagnostic assays for determining the level of expression of one or more biomarker proteins or nucleic acids, in order to determine whether an individual is at risk of developing immunosuppression or whether a vaccination has succeeded at providing protective immunity. Such assays can be used for prognostic or predictive purposes to thereby prophylactically treat or adjust the treatment regimen of an individual prior to the onset of the condition.

Yet another aspect of the invention pertains to monitoring the influence of agents, e.g., drugs or other compounds administered either to inhibit immunosuppression or to treat or prevent any other disorder e.g., chronic disorder (i.e., in order to understand any chronic effects that such treatment may have) on the expression or activity of a biomarker of the invention in clinical trials. The agents are described in further detail in the following sections.

The markers of the invention may serve as surrogate markers for one or more disorders or disease states or for conditions leading up to disease states, and in particular, prostate cancer. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., before pathogen exposure or early stage tumors), or when an assessment of capacity for adaptive immunity or disease progression is desired before a potentially dangerous clinical endpoint is reached (e.g., use of the biomarkers described herein or an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The markers of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker transcription or expression, the amplified marker may be in a quantity which is more readily detectable than the drug itself. Also, the marker may be more easily detected due to the nature of the marker itself; for example, using the methods described herein, antibodies may be employed in an immune-based detection system for a protein marker, or marker-specific radiolabeled nucleic acid probes may be used to detect a RNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J. Health-Syst. Pharm. 56 Suppl. 3: S16-S20.

Monitoring the influence of agents (e.g., drug compounds) on the level of expression of a marker of the invention can be applied not only in basic drug screening, but also in clinical trials or during treatment. For example, the effectiveness of an agent to affect marker expression can be monitored in clinical trials or subjects receiving treatment for chronic conditions. In a preferred embodiment, the present invention provides a method for monitoring the effectiveness of treatment of a subject with an agent, e.g., an immunomodulator (e.g., an agonist, antagonist, peptidomimetic, protein, peptide, nucleic acid, small molecule, or other drug candidate) comprising the steps of (i) obtaining a pre-administration sample from a subject prior to administration of the agent; (ii) detecting the level of expression of one or more selected markers of the invention in the pre-administration sample; (iii) obtaining one or more during and/or post-administration samples from the subject; (iv) detecting the level of expression of the marker(s) in the during and/or post-administration samples; (v) comparing the level of expression of the marker(s) in the pre-administration sample with the level of expression of the marker(s) in the during and/or post-administration sample or samples; and (vi) altering the administration of the agent to the subject accordingly. For example, increased expression of the biomarker during the course of treatment may effective vaccination or hyperplasia. Conversely, decreased expression of the biomarker may indicate immunosuppression caused by treatment and the desirability of decreasing the dosage, e.g., administering a lower dose and/or a less frequent administration schedule, e.g., at a dose statistically less than recommended for other patients receiving the treatment and/or at a dosing frequency less than recommended for patients receiving the treatment and optionally adding an agent which can treat the disorder by a different mechanism; or changing the therapeutic agent.

Many chronic conditions are treated with immunomodulators. There is risk of opportunistic infections for patients undergoing these treatments. Opportunistic infections are associated with significant morbidity and often are a challenge to treat. A simple, noninvasive test can monitor germinal center activity to identify risk of adverse events so treatment regimens can be adjusted accordingly.

Agents worth monitoring using this test include immunomodultors which include but are not limited to, anti-TNF agents, e.g., ENBREL® etanercept (Immunex Corp., Thousand Oaks, Calif.), REMICADE® infliximab (Centocor Inc., Malvern, Pa.), sulfasalazine) HUMIRA® adalimumab (Abbott Laboratories, Abbott Park, Ill.), CIMZIA® certolizumab pegol (CDP870, UCB S.A. Corp., Brussels, Belgium, a PEGylated Fab′ fragment of a humanized TNF inhibitor monoclonal antibody (its effects on B lymphocytes noted in Anolik et al. (2008) J. Immunol. 180:688-692)), CDP571; inhibitors of I kappa B kinase (IKK), e.g., IKKβ inhibitors, (e.g., beta-carbolines, e.g., N-(6-chloro-9H-beta-carbolin-8-yl) nicotinamide (PS-1145), U.S. Pat. Nos. 6,627,637, 7,026,331, N-(6-chloro-7-methoxy-9H-beta-carbolin-8-yl)-2-methyl-nicotinamide (ML120B, Nagashima et al. (2006) Blood 107:4266-73), U.S. Patent Application Publication No. 20040235839, Millennium Pharmaceuticals, Inc., Cambridge, Mass., BMS-345541, 4(2′-aminoethyl)amino-1,8-dimethylimidazo(1,2-a)quinoxaline, Bristol Myers Squibb, Princeton, N.J.); CD40 antagonists; CD40 ligands (e.g., TNX-100 anti-CD40 antibody, 5D12, effects on B lymphocytes noted in deVos et al. (2004) Eur. J. Immunol. 34:3446-55), RITUXAN® rituximab (Idec Pharmaceuticals Corp., San Diego, Calif.); anti-CD20 antibody (its effects on B lymphocytes noted in Nyman et al. (2007) Blood 109:4930-5, Anolik et al. (2007) Arthritis Rheum. 56:3044-56); steroidal antiinflammatory compounds, e.g., glucocorticoids (e.g., prednisone, prednisolone, adrenocorticotrophic hormone (ACTH), dexamethasone, methylprednisolone, hydrocortisone, triamcinolone, effects on B lymphocytes noted in Sackstein and Borenstein (1995) J. Invest. Med. 43:68-77, Scheinman et al. (1995) Science 270:283-6); immunosuppressive agents (e.g., azathioprene, 6-mercaptopurine, calcineurin inhibitors (e.g., cyclosporin A, effects on B lymphocytes noted in Kuper et al. (2007) Toxicol. Pathol. 35:226-32, Gore et al. (2008) Toxicology 197:23-35)); and other immunomodulators (e.g., thalidomide, interleukins (e.g., recombinant human IL-10, recombinant human IL-11, IL-2 antagonists (e.g., PROGRAF® tacrolimus, Apellas Pharma Inc., Tokyo, JP, FK-506; or antibodies against the IL-2 receptor alpha chain (CD25), e.g., SIMULECT® basiliximab, Novartis Pharmaceuticals Corp. East Hanover, N.J., or ZENAPAX® daclizumab, Hoffman-LaRoche Corp. Nutley, N.J.); CD-80 antagonists (e.g., ORENCIA® abatacept, Bristol-Myers Squibb, Princeton, N.J.); anti-IL-1 or other IL-1 antagonists (e.g., KINERET® anakinra, Amgen Inc., Thousand Oaks, Calif.); and signal transduction inhibitors (e.g., rapamycin, its effects on B lymphocytes noted in Woodland et al. (2008) Blood 111:750-60).

Additional agents whose effects, on germinal center activity can be tested or monitored include integrin antagonists, e.g., of alpha4beta1 integrin (α4β1 or VLA-4 antagonists, e.g., TYSABRI® natalizumab, Biogen Idec and Elan Corp., Dublin, IE, Cambridge, Mass. or firategrast, SB-683699, GlaxoSmithKline, London UK); of alpha4beta1 integrin (α4β7 antagonists, e.g., vedolizumab, Millennium Pharmaceuticals, Inc., Cambridge, Mass.); of beta2 integrin (β2 or CD18, antagonists, e.g., LFA-1 (CD11a/CD18) antagonists, e.g., RAPTIVA® efalizumab, Genentech, Inc., South San Francisco, Calif.); and proteasome inhibitors, e.g., peptidyl boronic acids, e.g., VELCADE® bortezomib, Millennium Pharmaceuticals, Inc., Cambridge, Mass.; peptide aldehydes, e.g., see U.S. Pat. No. 5,693,617; peptidyl epoxy ketones, e.g., see U.S. Pat. No. 6,831,099; alpha-ketoamides, see e.g., U.S. Pat. No. 6,310,057; or lactacystin and salinosporamide and analogs thereof, e.g., see U.S. Pat. No. 5,756,764, international patent publication WO 05/002572 or U.S. Pat. No. 7,276,530.

Examples of disorders being treated by the agents whose use can benefit from monitoring germinal center activity include autoimmune disorders (e.g., systemic lupus erythematosis, immune-mediated glomerulonephritis, insulin-dependent diabetes, multiple sclerosis, Hashimoto's thyroiditis, Grave's disease, and arthritis (e.g., juvenile rheumatoid arthritis, psoriatic arthritis, rheumatoid arthritis, ankylosing spondylitis, reactive arthritis, Lyme disease and osteoarthritis); disorders mediated by excess TNFα; inflammatory bowel disease, e.g., Crohn's disease and ulcerative colitis, sclerosing cholangitis, Celiac disease, pouchitis, eosinophilic gastroenteritis; skin disease, e.g., contact dermatitis, psoriasis and hidradenitis suppurativa; graft rejection, graft versus host disease, sarcoidosis, respiratory inflammatory diseases and disorders, such as asthma, chronic obstructive pulmonary disease and allergic rhinitis; gastrointestinal allergies, including food allergies, eosinophilia, conjunctivitis, glomerular nephritis; certain pathogen susceptibilities such as helminthic (e.g., leishmaniasis), certain viral infections, including HIV, and bacterial infections, including tuberculosis and lepromatous leprosy; cancers, e.g., B cell neoplasms (e.g., diffuse large B cell lymphoma, follicular lymphoma and B-cell prolymphocytic leukemia).

Opportunistic infections/diseases of which patients are at risk of acquiring: conditions caused by bacterial infection, e.g., tuberculosis, listerosis, pneumonia, shigellosis, and salmonella; viral infection, e.g., infection by herpesvirus, poxvirus, and adenovirus, cytomegalovirus, varicella zoster virus or Epstein-Barr virus. Certain disorders are associated with an increased number of surviving cells, which are produced and continue to survive or proliferate when apoptosis is inhibited or occurs at an undesirably low rate. These disorders include cancer (particularly follicular lymphomas, plasmablastic lymphoma, chronic myelogenous leukemia, multiple myeloma, mycosis fungoides, melanoma, colon cancer, lung carcinoma, carcinomas associated with mutations in p53, and hormone-dependent tumors such as breast cancer, prostate cancer, and ovarian cancer). Failure to remove autoimmune cells that arise during development or that develop as a result of somatic mutation during an immune response can result in autoimmune disease. Thus, an autoimmune disorder (e.g., systemic lupus erythematosis, immune-mediated glomerulonephritis, and arthritis) can be caused by an undesirably low level of apoptosis.

The invention also includes a method of assessing the efficacy of a test compound for modulating adaptive immunity. As described above, differences in the level of expression of the markers of the invention correlate with the capacity of adaptive immunity. It is recognized that changes in the levels of expression of some of the biomarkers of the invention indicate isotype class switching. Thus, compounds which affect adaptive immunity in a patient, e.g., one with immunosuppression or one who received a vaccine, will cause the level of expression of one or more of the biomarkers of the invention to change to a level different than the normal level of expression for that marker (i.e., the level of expression for the marker in mature B cells or germline B cells, respectively).

This method thus comprises comparing the expression of a marker in a first sample comprising B cells and maintained in the presence of the test compound and expression of the marker in a second sample comprising B cells and maintained in the absence of the test compound. A significantly reduced expression of a marker of the invention in the presence of the test compound is an indication that the test compound inhibits B cell activity. The samples may, for example, be aliquots of a single sample comprising normal B cells obtained from a patient, pooled samples comprising normal B cells obtained from a patient, cells of a normal B cell line, aliquots of a single sample comprising B cells obtained from a patient, pooled samples comprising B cells obtained from a patient, cells of B cell line, or the like. In one embodiment, the samples comprising B cells are obtained from a patient and a plurality of compounds known to be effective for inhibiting various B cell activities are tested in order to identify the compound which is most likely to induce immunization or not to induce immunosuppression in the patient.

This method likewise may be used to assess the efficacy of a therapy for treating chronic disease in a patient or potential for the therapy to cause adverse effects. In this method, the level of expression of one or more markers of the invention in a pair of samples comprising B cells (one subjected to the therapy, the other not subjected to the therapy) is assessed. As with the method of assessing the efficacy of test compounds, if the therapy induces a significantly lower level of expression of a marker of the invention then the therapy can cause germinal center atrophy. As above, if samples from a selected patient are used in this method, then alternative therapies can be assessed in vitro in order to select a therapy most likely to be efficacious for modulating adaptive immunity in the patient. Conversely, if samples from a selected patient are used in this method, then alternative therapies can be assessed in vitro in order to select a therapeutic agent or dose least likely to cause detrimental immunosuppression.

### Detection Methods

A general principle of such diagnostic and prognostic assays involves preparing a sample or reaction mixture that may contain a marker, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay. One example of such an embodiment includes use of an array or chip which contains a predictive marker or marker set anchored for expression analysis of the sample.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, nylon, polyethylene, dextran, amylases, natural and modified celluloses; polyacrylamides, gabbros, and magnetite. One skilled in the art will know many other suitable carriers for binding antibody or antigen, and will be able to adapt such support for use with the present invention. For example, protein isolated from blood cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein.

In a preferred embodiment, the probe, when it is the unanchored assay component, can be labeled for the purpose of detection and readout of the assay, either directly or indirectly, with detectable labels discussed herein and which are well-known to one skilled in the art. The term “labeled”, with regard to the probe (e.g., nucleic acid or antibody), is intended to encompass direct labeling of the probe by coupling (i.e., physically linking) a detectable substance to the probe, as well as indirect labeling of the probe by reactivity with another reagent that is directly labeled. An example of indirect labeling includes detection of a primary antibody using a fluorescently labeled secondary antibody. It is also possible to directly detect marker/probe complex formation without further manipulation or labeling of either component (marker or probe), for example by utilizing the technique of fluorescence energy transfer (FET, see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). As used herein, “BIA” or “surface plasmon resonance” is a technology for studying biospecific interactions in real time, without labeling any of the interactants (e.g., BIACORE™). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas, G., and Minton, A. P. (1993) Trends Biochem Sci. 18:284-7). Standard chromatographic techniques also can be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H. (1998) J. Mol. Recognit. 11: 141-8; Hage, D. S., and Tweed, S. A. (1997) J. Chromatogr. B. Biomed. Appl. 699:499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

The isolated mRNA can be used in hybridization or amplification assays that include, but are not limited to, Southern or Northern analyses, polymerase chain reaction and TAQMAN® gene expression assays (Applied Biosystems, Foster City, Calif.) and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 20, 25, 30, 50, 75, 100, 125, 150, 175, 200, 250 or 500 or more consecutive nucleotides of the biomarker transcript and sufficient to specifically hybridize under stringent conditions to a mRNA or genomic DNA encoding a marker of the present invention. The exact length of the nucleic acid probe will depend on many factors that are routinely considered and practiced by the skilled artisan. Nucleic acid probes of the invention may be prepared by chemical synthesis using any suitable methodology known in the art, may be produced by recombinant technology, or may be derived from a biological sample, for example, by restriction digestion. Other suitable probes for use in the diagnostic assays of the invention are described herein. The probe can comprise a label group attached thereto, e.g., a radioisotope, a fluorescent compound, an enzyme, an enzyme co-factor, a hapten, a sequence tag, a protein or an antibody. The nucleic acids can be modified at the base moiety, at the sugar moiety, or at the phosphate backbone. An example of a nucleic acid label is incorporated using SUPER™ Modified Base Technology (Nanogen, Bothell, Wash., see U.S. Pat. No. 7,045,610). The level of expression can be measured as general nucleic acid levels, e.g., after measuring the amplified DNA levels (e.g. using a DNA intercalating dye, e.g., the SYBR green dye (Qiagen Inc., Valencia, Calif.) or as specific nucleic acids, e.g., using a probe based design, with the probes labeled. Preferable TAQMAN® assay formats use the probe-based design to increase specificity and signal-to-noise ratio.

Such probes can be used as part of a diagnostic test kit for identifying cells or tissues which express the protein, such as by measuring levels of a nucleic acid molecule transcribed during ICS or encoding a DNA recombination effector protein in a sample of cells from a subject, e.g., detecting transcript, mRNA levels or determining whether a gene encoding the protein has been mutated or deleted. Hybridization of an RNA or a cDNA with the nucleic acid probe indicates that the marker in question is being expressed. The invention further encompasses detecting nucleic acid molecules that differ, due to degeneracy of the genetic code, from the nucleotide sequence of nucleic acids encoding a marker protein (e.g., protein having the sequence of the SEQ ID NOs:4, 6, 8, 10, 12, 14, or 16), and thus encode the same protein. It will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Detecting any and all such nucleotide variations and resulting amino acid polymorphisms or variations that are the result of natural allelic variation and that do not alter the functional activity are intended to be within the scope of the invention. In addition, it will be appreciated that DNA polymorphisms that affect RNA expression levels can also exist that may affect the overall expression level of that gene (e.g., by affecting regulation or degradation).

The nucleic acids of the invention can be single stranded DNA (e.g., an oligonucleotide), double stranded DNA (e.g., double stranded oligonucleotide) or RNA. Preferred nucleic acids of the invention can be used as probes or primers. Primers of the invention refer to nucleic acids which hybridize to a nucleic acid sequence which is adjacent to the region of interest and is extended or which covers the region of interest. As used herein, the term “hybridizes” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85%, 90% or 95% identical to each other remain hybridized to each other for subsequent amplification and/or detection. Stringent conditions vary according to the length of the involved nucleotide sequence but are known to those skilled in the art and can be found or determined based on teachings in Current Protocols in Molecular Biology, Ausubel et al., eds., John Wiley & Sons, Inc. (1995), sections 2, 4 and 6. Additional stringent conditions and formulas for determining such conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions for hybrids that are at least 10 basepairs in length includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions for such hybrids includes hybridization in 1×SSC, at about 65-70° C. (or hybridization in IX SSC plus 50% formamide at about 42-50° C.) followed by one or more washes in 0.3×SSC, at about 65-70° C. A preferred, non-limiting example of reduced stringency hybridization conditions for such hybrids includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. Another example of stringent hybridization conditions are hybridization in 6× sodium chloride/sodium citrate (SSC) at about 45° C., followed by one or more washes in 0.2×SSC, 0.1% SDS at 50-65° C. A further example of stringent hybridization buffer is hybridization in 1 M NaCl, 50 mM 2-(N-morpholino)ethanesulfonic acid (MES) buffer (pH 6.5), 0.5% sodium sarcosine and 30% formamide. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀ [Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, polyvinylpyrrolidone (PVP) and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS). A primer or nucleic acid probe can be used alone in a detection method, or a primer can be used together with at least one other primer or nucleic acid probe in a detection method. Primers can also be used to amplify at least a portion of a nucleic acid. Nucleic acid probes of the invention refer to nucleic acids which hybridize to the region of interest and which are not further extended. For example, a nucleic acid probe is a nucleic acid which specifically hybridizes to a polymorphic region of a biomarker, and which by hybridization or absence of hybridization to the DNA of a patient or the type of hybrid formed will be indicative of the identity of the allelic variant of the polymorphic region of the biomarker or the amount of germinal center activity.

In one format, the RNA is immobilized on a solid surface and contacted with a probe, for example by running the isolated RNA on an agarose gel and transferring the RNA from the gel to a membrane, such as nitrocellulose. In an alternative format, the nucleic acid probe(s) are immobilized on a solid surface and the RNA is contacted with the probe(s), for example, in an AFFYMETRIX® gene chip array (Santa Clara, Calif.) or customized array using a biomarker set comprising at least one biomarker indicative of ICS or B cell clonal expansion. A skilled artisan can readily adapt known RNA detection methods for use in detecting the level of RNA encoded by the markers of the present invention. For example, the high density microarray or branched DNA assay can benefit from a higher concentration of B cell in the sample, such as a sample which had been modified to isolate B cells as described in earlier sections. In a related embodiment, a mixture of transcribed polynucleotides obtained from the sample is contacted with a substrate having fixed thereto a polynucleotide complementary to or homologous with at least a portion (e.g., at least 7, 10, 15, 20, 25, 30, 40, 50, 100, 500, or more nucleotide residues) of a marker nucleic acid. If polynucleotides complementary to or homologous with the marker are differentially detectable on the substrate (e.g., detectable using different chromophores or fluorophores, or fixed to different selected positions), then the levels of expression of a plurality of markers can be assessed simultaneously using a single substrate (e.g., a “gene chip” microarray of polynucleotides fixed at selected positions). When a method of assessing marker expression is used which involves hybridization of one nucleic acid with another, it is preferred that the hybridization be performed under stringent hybridization conditions.

An alternative method for determining the level of RNA corresponding to a marker of the present invention in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to about 30 nucleotides in length and flank a region from about 50 to about 200 nucleotides in length. Under appropriate conditions and with appropriate reagents, such primers permit the amplification of a nucleic acid molecule comprising the nucleotide sequence flanked by the primers.

For in situ methods, RNA does not need to be isolated from the cells prior to detection. In such methods, a cell or tissue sample is prepared/processed using known histological methods. The sample is then immobilized on a support, typically a glass slide, and then contacted with a probe that can hybridize to RNA that encodes the marker.

In another embodiment of the present invention, a polypeptide corresponding to a marker is detected. A preferred agent for detecting a polypeptide of the invention is an antibody capable of binding to a polypeptide corresponding to a marker of the invention, preferably an antibody with a detectable label. Antibodies can be polyclonal, or more preferably, monoclonal. An intact antibody, or a fragment thereof (e.g., Fab or F(ab′)₂) can be used.

A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining whether B cells express a marker of the present invention.

Another method for determining the level of a polypeptide corresponding to a marker is mass spectrometry. For example, intact proteins or peptides, e.g., tryptic peptides can be analyzed from a sample, e.g., a blood sample, a lymph sample or other sample, containing one or more polypeptide markers. The method can further include treating the sample to lower the amounts of abundant proteins, e.g., serum albumin, to increase the sensitivity of the method. For example, liquid chromatography can be used to fractionate the sample so portions of the sample can be analyzed separately by mass spectrometry. The steps can be performed in separate systems or in a combined liquid chromatography/mass spectrometry system (LC/MS, see for example, Liao, et al. (2004) Arthritis Rheum. 50:3792-3803). The mass spectrometry system also can be in tandem (MS/MS) mode. The charge state distribution of the protein or peptide mixture can be acquired over one or multiple scans and analyzed by statistical methods, e.g. using the retention time and mass-to-charge ratio (m/z) in the LC/MS system, to assess germinal center activity or capacity for adaptive immunity, including testing samples from patients responsive or non-responsive to proteasome inhibition and/or glucocorticoid therapy. Examples of mass spectrometers which can be used are an ion trap system (ThermoFinnigan, San Jose, Calif.) or a quadrupole time-of-flight mass spectrometer (Applied Biosystems, Foster City, Calif.). The method can further include the step of peptide mass fingerprinting, e.g. in a matrix-assisted laser desorption ionization with time-of-flight (MALDI-TOF) mass spectrometry method. The method can further include the step of sequencing one or more of the tryptic peptides. Results of this method can be used to identify proteins from primary sequence databases, e.g., maintained by the National Center for Biotechnology Information, Bethesda, Md., or the Swiss Institute for Bioinformatics, Geneva, Switzerland, and based on mass spectrometry tryptic peptide m/z base peaks.

### Electronic Apparatus Readable Arrays

Electronic apparatus, including readable arrays comprising at least one predictive marker of the present invention is also contemplated for use in conjunction with the methods of the invention. As used herein, “electronic apparatus readable media” refers to any suitable medium for storing, holding or containing data or information that can be read and accessed directly by an electronic apparatus. As used herein, the term “electronic apparatus” is intended to include any suitable computing or processing apparatus or other device configured or adapted for storing data or information. Examples of electronic apparatus suitable for use with the present invention and monitoring of the recorded information include stand-alone computing apparatus; networks, including a local area network (LAN), a wide area network (WAN) Internet, Intranet, and Extranet; electronic appliances such as personal digital assistants (PDAs), cellular phone, pager and the like; and local and distributed processing systems. As used herein, “recorded” refers to a process for storing or encoding information on the electronic apparatus readable medium. Those skilled in the art can readily adopt any of the presently known methods for recording information on known media to generate manufactures comprising the markers of the present invention.

For example, microarray systems are well known and used in the art for assessment of samples, whether by assessment gene expression (e.g., RNA detection, protein detection), or metabolite production, for example. Microarrays for use according to the invention include one or more probes of predictive marker(s) of the invention characteristic of response and/or non-response to a therapeutic regimen as described herein. In one embodiment, the microarray comprises one or more probes corresponding to one or more of markers selected from the group consisting of markers which demonstrate increased expression in short term survivors, and genes which demonstrate increased expression in long term survivors in patients. A number of different microarray configurations and methods for their production are known to those of skill in the art and are disclosed, for example, in U.S. Pat. Nos. 5,242,974; 5,384,261; 5,405,783; 5,412,087; 5,424,186; 5,429,807; 5,436,327; 5,445,934; 5,556,752; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,624,711; 5,700,637; 5,744,305; 5,770,456; 5,770,722; 5,837,832; 5,856,101; 5,874,219; 5,885,837; 5,919,523; 5,981,185; 6,022,963; 6,077,674; 6,156,501; 6,261,776; 6,346,413; 6,440,677; 6,451,536; 6,576,424; 6,610,482; 5,143,854; 5,288,644; 5,324,633; 5,432,049; 5,470,710; 5,492,806; 5,503,980; 5,510,270; 5,525,464; 5,547,839; 5,580,732; 5,661,028; 5,848,659; and U.S. Pat. No. 5,874,219; Shena, et al. (1998), Tibtech 16:301; Duggan et al. (1999) Nat. Genet. 21:10; Bowtell et al. (1999) Nat. Genet. 21:25; Lipshutz et al. (1999) Nature Genet. 21:20-24, 1999; Blanchard, et al. (1996) Biosensors and Bioelectronics, 11:687-90; Maskos, et al., (1993) Nucleic Acids Res. 21:4663-69; Hughes, et al. (2001) Nat. Biotechol. 19:342, 2001; each of which are herein incorporated by reference. A tissue microarray can be used for protein identification (see Hans et al. (2004) Blood 103:275-282). A phage-epitope microarray can be used to identify one or more proteins in a sample based on whether the protein or proteins induce auto-antibodies in the patient (Bradford et al. (2006) Urol. Oncol. 24:237-242).

A microarray thus comprises one or more probes corresponding to one or more biomarkers identified herein, e.g., those indicative of ICS or germinal center activity. The microarray can comprise probes corresponding to, for example, at least 2, at least 5, at least 10, at least 15, at least 20, at least 25, at least 30, at least 35, at least 40, at least 45, at least 50, at least 75, or at least 100, biomarkers indicative of ICS or germinal center activity. The microarray can comprise probes corresponding to one or more biomarkers as set forth herein. Still further, the microarray may comprise complete marker sets as set forth herein and which may be selected and compiled according to the methods set forth herein. The microarray can be used to assay expression of one or more predictive markers or predictive marker sets in the array. In one example, the array can be used to assay more than one predictive marker or marker set expression in a sample to ascertain an expression profile of markers in the array. In this manner, up to about 44,000 markers can be simultaneously assayed for expression. This allows an expression profile to be developed showing a battery of markers specifically expressed in one or more samples. Still further, this allows an expression profile to be developed to assess capacity for adaptive immunity or degree of immunosuppression.

The array is also useful for ascertaining differential expression patterns of one or more markers in normal and affected (e.g., blood, e.g., B) cells. This provides a battery of predictive markers that could serve as a tool for ease of identification of subjects with affected adaptive immunity, e.g., those with atrophic or hyperplastic germinal centers. Further, the array is useful for ascertaining expression of reference markers for reference expression levels. In another example, the array can be used to monitor the time course of expression of one or more bio markers in the array.

In addition to such qualitative determination, the invention allows the quantification of marker expression. Thus, predictive markers can be grouped on the basis of marker sets or short term or long term indications by the level of expression in the sample. This is useful, for example, in ascertaining the short term or long term indication of adaptive immunity in the sample by virtue of scoring the expression levels according to the methods provided herein.

The array is also useful for ascertaining the effect of the expression of a marker on the expression of other biomarkers in the same cell or in different cells. This provides, for example, a selection of alternate molecular targets for therapeutic intervention if a patient is predicted to be adversely affected, e.g., immunosuppressed by the test agent.

### Reagents and Kits

The invention also encompasses kits for detecting the presence of a polypeptide or nucleic acid corresponding to a marker of the invention in a biological sample (e.g. an immune system-associated body fluid such as a blood sample). Such kits can be used to assess capacity for adaptive immunity, e.g., determine if a subject is at increased risk of developing immunosuppression or has increased adaptive immunity. For example, the kit can comprise a labeled compound or agent capable of detecting a polypeptide or a transcribed RNA corresponding to a marker of the invention in a biological sample and means for determining the amount of the polypeptide or RNA in the sample. Suitable reagents for binding with a marker protein include antibodies, antibody derivatives, antibody fragments, and the like. Suitable reagents for binding with a marker nucleic acid (e.g., a genomic DNA, an mRNA, a spliced mRNA, a cDNA, or the like) include complementary nucleic acids. The kit can also contain a control or reference sample or a series of control or reference samples which can be assayed and compared to the test sample. For example, the kit may have a positive control sample, e.g., including one or more biomarkers described herein, or reference markers, e.g. housekeeping markers to standardize the assay among samples or timepoints. By way of example, the kit may comprise fluids (e.g., buffer) suitable for annealing complementary nucleic acids or for binding an antibody with a protein with which it specifically binds and one or more sample compartments. The kit of the invention may optionally comprise additional components useful for performing the methods of the invention, e.g., a sample collection vessel, e.g., a tube, and optionally, means for optimizing the amount of biomarker detected, for example if there may be time or adverse storage and handling conditions between the time of sampling and the time of analysis. For example, the kit can contain means for increasing the number of B cells in the sample, as described above, a buffering agent, a preservative, a stabilizing agent or additional reagents for preparation of cellular material or probes for use in the methods provided; and detectable label, alone or conjugated to or incorporated within the provided probe(s). In one exemplary embodiment, a kit comprising a sample collection vessel can comprise e.g., a tube comprising anti-coagulant and/or stabilizer, as described above, or known to those skilled in the art. The kit can further comprise components necessary for detecting the detectable label (e.g., an enzyme or a substrate). For marker sets, the kit can comprise a marker set array or chip for use in detecting the biomarkers. Kits also can include instructions for interpreting the results obtained using the kit. The kit can contain reagents for detecting one or more biomarkers, e.g., 2, 3, 4, 5, or more biomarkers described herein.

In one embodiment, the kit comprises a probe to detect at least one biomarker, e.g., a marker indicative of ICS (e.g., a germline switch transcript, a circle transcript and a DNA recombination effector). In an exemplary embodiment, the kit comprises a probe to detect a biomarker selected from the group consisting of SEQ ID NOs:1-21, 58, 59 and 60 (i.e., to detect a biomarker selected from the group consisting of SEQ ID NOs:1, 2, 3, 4, 5, 6, 7, 8, 9, 10, 11, 12, 13, 14, 15, 16, 17, 18, 19, 20, 21, 58, 59 and 60). In preferred embodiments, the kit comprises a probe to detect a biomarker selected from the group consisting of GLT-μ, AICDA (AID), CT-γ1&2, IGHG1 and/or IGHA2. In related embodiments, the kit comprises a nucleic acid probe comprising or derived from (e.g., a fragment or variant (e.g., homologous or complementary) thereof) a nucleic acid sequence selected from the group consisting of SEQ ID NOs:22-57. In preferred embodiments, the kit comprises a nucleic acid probe comprising or derived from (e.g., a fragment or variant (e.g., homologous or complementary) thereof) a nucleic acid sequence selected from the group consisting of SEQ ID NOs: 53, 55, 34, 36, 35, 54, 37, 56, and 57.

For kits comprising nucleic acid probes, e.g., oligonucleotide-based kits, the kit can comprise, for example: one or more nucleic acid reagents such as an oligonucleotide (labeled or non-labeled) which hybridizes to a nucleic acid sequence corresponding to a marker of the invention, optionally fixed to a substrate; labeled oligonucleotides not bound with a substrate, a pair of PCR primers, useful for amplifying a nucleic acid molecule corresponding to a marker of the invention, molecular beacon probes, a marker set comprising oligonucleotides which hybridize to at least two nucleic acid sequences corresponding to markers of the invention, and the like. The kit can contain an RNA-stabilizing agent.

For kits comprising protein probes, e.g., antibody-based kits, the kit can comprise, for example: (1) a first antibody (e.g., attached to a solid support) which binds to a polypeptide corresponding to a marker of the invention; and, optionally, (2) a second, different antibody which binds to either the polypeptide or the first antibody and is conjugated to a detectable label. The kit can contain a protein stabilizing agent. The kit can contain reagents to reduce the amount of non-specific binding of non-biomarker material from the sample to the probe. Examples of reagents include nonioinic detergents, non-specific protein containing solutions, such as those containing albumin or casein, or other substances known to those skilled in the art.

An isolated polypeptide corresponding to a predictive marker of the invention, or a fragment thereof, can be used as an immunogen to generate antibodies using standard techniques for polyclonal and monoclonal antibody preparation. For example, an immunogen typically is used to prepare antibodies by immunizing a suitable (i.e., immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Antibodies include immunoglobulin molecules and immunologically active portions of immunoglobulin molecules, i.e., molecules that contain an antigen binding site which specifically binds an antigen, such as a polypeptide of the invention, e.g., an epitope of a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab)₂ fragments which can be generated by treating the antibody with an enzyme such as pepsin. The invention provides polyclonal and monoclonal antibodies. Synthetic and genetically engineered variants (See U.S. Pat. No. 6,331,415) of any of the foregoing are also contemplated by the present invention. Polyclonal and monoclonal antibodies can be produced by a variety of techniques, including conventional murine monoclonal antibody methodology e.g., the standard somatic cell hybridization technique of Kohler and Milstein, Nature 256: 495 (1975) the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. See generally, Harlow, E. and Lane, D. (1988) Antibodies: A Laboratory Manual, Cold Spring Harbor Laboratory Press, Cold Spring Harbor, N.Y.; and Current Protocols in Immunology, Coligan et al. ed., John Wiley & Sons, New York, 1994. Preferably, for diagnostic applications, the antibodies are monoclonal antibodies. Additionally, for use in in vivo applications the antibodies of the present invention are preferably human or humanized antibodies. Hybridoma cells producing a monoclonal antibody of the invention are detected by screening the hybridoma culture supernatants for antibodies that bind the polypeptide of interest, e.g., using a standard ELISA assay.

If desired, the antibody molecules can be harvested or isolated from the subject (e.g., from the blood or serum of the subject) and further purified by well-known techniques, such as protein A chromatography to obtain the IgG fraction. Alternatively, antibodies specific for a protein or polypeptide of the invention can be selected or (e.g., partially purified) or purified by, e.g., affinity chromatography to obtain substantially purified and purified antibody. By a substantially purified antibody composition is meant, in this context, that the antibody sample contains at most only 30% (by dry weight) of contaminating antibodies directed against epitopes other than those of the desired protein or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein or polypeptide of the invention.

An antibody directed against a polypeptide corresponding to a marker of the invention (e.g., a monoclonal antibody) can be used to detect the marker (e.g., in a cellular sample) in order to evaluate the level and pattern of expression of the marker. The antibodies can also be used diagnostically to monitor protein levels in tissues or body fluids (e.g. in a blood sample) as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Accordingly, in one aspect, the invention provides substantially purified antibodies or fragments thereof, and non-human antibodies or fragments thereof, which antibodies or fragments specifically bind to a polypeptide comprising an amino acid sequence encoded by a marker identified herein. The substantially purified antibodies of the invention, or fragments thereof, can be human, non-human, chimeric and/or humanized antibodies.

In another aspect, the invention provides non-human antibodies or fragments thereof, which antibodies or fragments specifically bind to a polypeptide comprising an amino acid sequence which is encoded by a nucleic acid molecule of a predictive marker of the invention. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

The substantially purified antibodies or fragments thereof may specifically bind to a signal peptide, a secreted sequence, an extracellular domain, a transmembrane or a cytoplasmic domain or cytoplasmic membrane of a polypeptide of the invention. The substantially purified antibodies or fragments thereof, the non-human antibodies or fragments thereof, and/or the monoclonal antibodies or fragments thereof, of the invention specifically bind to a secreted sequence or an extracellular domain of the amino acid sequences of the present invention.

The invention also provides a kit containing an antibody of the invention conjugated to a detectable substance, and instructions for use. Still another aspect of the invention is a diagnostic composition comprising a probe of the invention and a pharmaceutically acceptable carrier. In one embodiment, the diagnostic composition contains an antibody of the invention, a detectable moiety, and a pharmaceutically acceptable carrier.

### Use of Information

The methods for processing approval of payment or processing of payment for a treatment regimen of a patient receiving an immunomodulator, e.g., a regimen which increases or decreases ICS as described herein, include a step of reviewing the patient's biomarker expression levels, a further step of making a decision or advising on whether payment should be made for the treatment regimen based on the result of the evaluation, and a further step of transmitting or recording the decision or advice.

In one method, information, e.g., about the patient's marker expression levels (e.g., the result of evaluating a biomarker or biomarker set described herein), or about whether a patient is expected to have beneficial or detrimental effects on immunocompetence, is provided (e.g., communicated, e.g., electronically communicated) to a third party, e.g., a hospital, clinic, a government entity, reimbursing party or insurance company (e.g., a life insurance company). For example, choice of medical procedure, payment for a medical procedure, payment by a reimbursing party, or cost for a service or insurance can be function of the information. For example, the third party receives the information, makes a determination based at least in part on the information, and optionally communicates the information or makes a choice of procedure, payment, level of payment, coverage, etc. based on the information. In the method, the expression profile or trend in expression level of a biomarker or a biomarker set selected from or derived from the biomarkers described herein is determined.

In one embodiment, a premium for insurance (e.g., life or medical) is evaluated as a function of information about one or more marker expression levels, e.g., a biomarker or biomarker set, e.g., a level of expression associated with have beneficial or detrimental effects on immunocompetence (e.g., the trend in expression level or expression profile). For example, premiums can be increased (e.g., by a certain percentage) if the biomarkers of a patient or a patient's biomarker set described herein are differentially expressed between an insured candidate (or a candidate seeking insurance coverage) and a reference value (e.g., a non-afflicted person or nontreated person). Premiums can also be scaled depending on marker expression levels, e.g., the result of evaluating a biomarker or biomarker set described herein. For example, premiums can be assessed to distribute risk, e.g., as a function of marker expression levels, e.g., the result of evaluating a biomarker or biomarker set described herein. In another example, premiums are assessed as a function of actuarial data that is obtained from patients that have beneficial or detrimental effects on immunocompetence.

Information about marker expression levels, e.g., the result of evaluating a biomarker or biomarker set described herein (e.g., the trend in expression level or expression profile), can be used, e.g., in an underwriting process for life insurance. The information can be incorporated into a profile about a subject. Other information in the profile can include, for example, date of birth, gender, marital status, banking information, credit information, children, and so forth. An insurance policy can be recommended as a function of the information on marker expression levels, e.g., the result of evaluating a predictive marker or predictive marker set described herein, along with one or more other items of information in the profile. An insurance premium or risk assessment can also be evaluated as function of the biomarker or biomarker set information. In one implementation, points are assigned on the basis of a result showing beneficial or detrimental effects on immunocompetence.

In one embodiment, information about marker expression levels, e.g., the result of evaluating a biomarker or biomarker set described herein, is analyzed by a function that determines whether to authorize the transfer of funds to pay for a service or treatment provided to a subject (or make another decision referred to herein). For example, the results of analyzing a expression of a biomarker or biomarker set described herein may indicate that a subject has beneficial or detrimental effects on immunocompetence, suggesting that a treatment course is needed, thereby triggering an outcome that indicates or causes authorization to pay for a service or treatment provided to a subject. In one example, an expression profile or a trend in the expression level of a biomarker or a biomarker set selected from or derived from the biomarkers described herein is determined and payment is authorized if the expression profile or trend in expression level identifies a beneficial effect on immunocompetence. For example, an entity, e.g., a hospital, care giver, government entity, or an insurance company or other entity which pays for, or reimburses medical expenses, can use the outcome of a method described herein to determine whether a party, e.g., a party other than the subject patient, will pay for services (e.g., a particular therapy) or treatment provided to the patient. For example, a first entity, e.g., an insurance company, can use the outcome of a method described herein to determine whether to provide financial payment to, or on behalf of, a patient, e.g., whether to reimburse a third party, e.g., a vendor of goods or services, a hospital, physician, or other care-giver, for a service or treatment provided to a patient. For example, a first entity, e.g., an insurance company, can use the outcome of a method described herein to determine whether to continue, discontinue, enroll an individual in an insurance plan or program, e.g., a health insurance or life insurance plan or program.

In one aspect, the disclosure features a method of providing data. The method includes providing data described herein, e.g., generated by a method described herein, to provide a record, e.g., a record described herein, for determining if a payment will be provided. In some embodiments, the data is provided by computer, compact disc, telephone, facsimile, email, or letter. In some embodiments, the data is provided by a first party to a second party. In some embodiments, the first party is selected from the subject, a healthcare provider, a treating physician, a health maintenance organization (HMO), a hospital, a governmental entity, or an entity which sells or supplies the drug. In some embodiments, the second party is a third party payor, an insurance company, employer, employer sponsored health plan, HMO, or governmental entity. In some embodiments, the first party is selected from the subject, a healthcare provider, a treating physician, an HMO, a hospital, an insurance company, or an entity which sells or supplies the drug and the second party is a governmental entity. In some embodiments, the first party is selected from the subject, a healthcare provider, a treating physician, an HMO, a hospital, an insurance company, or an entity which sells or supplies the drug and the second party is an insurance company.

In another aspect, the disclosure features a record (e.g., computer readable record) which includes a list and value of expression for the biomarker or biomarker set for a patient. In some embodiments, the record includes more than one value for each marker.

The present invention will now be illustrated by the following Examples, which are not intended to be limiting in any way

### EXAMPLES

### Example 1

### Measurement of ICS in Mice

Biomarker transcripts were examined for their potential as surrogate measures of splenic germinal center atrophy upon treatment with a specific I Kappa B Kinase Beta (IKKβ) inhibitor, a beta-carboline. Splenic germinal center atrophy in mice can be due to inhibition of IKKβ in B lymphocytes, because targeted deletion of the IKKβ gene in B lymphocytes in mice induces a similar phenotype (Pasparakis et al. (2002) J. Exp. Med 196:743-52, Li et al. (2003) J. Immunol. 170:4630-7, Ren et al. (2002) J. Immunol. 168:577-87). Expression of an endogenous reference transcript (18S), which is ubiquitously expressed and not regulated by the NF-κB pathway, also was measured, to allow normalization of the transcript data.

Female C57BL/6 mice (Charles River Laboratories, Bedford, Mass.) were used in these studies and treated with a beta-carboline, N-(6-chloro-7-methoxy-9H-beta-carbolin-8-yl)-2-methyl-nicotinamide (ML120B) following three different treatment regimens, A, B, and C. After the treatment, animals were euthanized and tissues were collected and frozen. Germinal centers were identified by immunohistochemisry (IHC) of cryosections (6 μm) using an antibody to mouse Bcl-6. T cells also were identified by IHC of serial cryosections, using an antibody to mouse CD3. The size and/or frequency of germinal centers and T cells were scored by a board-certified anatomic pathologist, using a 0 (low) to 4 (high) scale. Additionally, the samples were analyzed by qRT-PCR using the nucleic acid probes and primers listed in Table 1.

TABLE 1
Mouse Quantitative Reverse-Transcriptase Polymerase Chain Reaction
Reagent Sequences

| | Reference | | |
|---|---|---|---|
| | Sequence | | |
| Assay | ID | Oligomer | Sequence |
| Mu-GLT | NC_000078 | Forward | 5′-CTCTGGCCCTGCTTATTGTTG-3′ SEQ ID NO: 22 |
| | | Reverse | 5′-GAAGACATTTGGGAAGGACTGACT-3′ SEQ ID NO: 23 |
| G3-GLT | NC_000078 | Forward | 5′-TGGGCAAGTGGATCTGAACA-3′ SEQ ID NO: 24 |
| | | Reverse | 5′-CTCAGGGAAGTAGCCTTTGACA-3′ SEQ ID NO: 25 |
| G1-GLT | NC_000078 | Forward | 5′-GGCCCTTCCAGATCTTTGAG-3′ SEQ ID NO: 26 |
| | | Reverse | 5′-GGATCCAGAGTTCCAGGTCACT-3′SEQ ID NO: 27 |
| | NG_001019 | Forward | 5′-GGGCTTCCAAGCCAACAGGGCAGGACA-3′ SEQ ID NO: 28 |
| | | Reverse | 5′-GTTGCCGTTGGGGTGCTGGAC-3′ SEQ ID NO: 29 |
| AICDA-s | NM_009645.2 | Forward | 5′-GGCTGAGGTTAGGGTTCCATCTCAG-3′ SEQ ID NO: 30 |
| | | Reverse | 5′-GAGGGAGTCAAGAAAGTCACGCTGGA-3′ SEQ ID NO: 31 |
| 18S | X03205.1 | Forward | 5′-GTTAGCATGCCAGAGTCTCGTTCGTT-3′ SEQ ID NO: 32 |
| | | Reverse | 5′-CCGTTCTTAGTTGGTGG-3′ SEQ ID NO: 33 |

Mu-GLT = Mu germline transcript; G1-GLT = Gamma 1 germline transcript; G3-GLT = Gamma 3 germline transcript; CT-γ1&2 = circle transcripts containing gamma 1 and 2; AICDA = activation-induced cytidine deaminase.

The following two paragraphs provide methods for qRT-PCR:

Total ribonucleic acid (RNA) was isolated from 10 spleen cryosections, each 6 using the RNEASY® Universal Tissue 8000 Kit (QIAGEN Inc., Valencia, Calif.) on a BIOROBOT® 8000 Workstation (QIAGEN INC., Valencia, Calif.), and deoxyribonucleic acidase (DNASE)-treated according to the manufacturer's protocol. The purity and yield of the RNA were assessed via spectrophotometry from 220 to 750 nm using the NANODROP® ND-1000 (NANODROP Technologies, Wilmington, Del.). The integrity of the RNA was measured with the RNA 6000 NANO LABCHIP® Kit (AGILENT Technologies, Foster City, Calif.) on an AGILENT 2100 Bioanalyzer (AGILENT Technologies, Foster City, Calif.) and calculated using the RNA integrity number (RIN) algorithm (Schroeder et al. (2006) BMC Mol. Biol. 7:3). Deoxyribonucleic acidase-treated RNA was extracted from the samples and stored at −80° C. First strand complementary deoxyribonucleic acid (cDNA) synthesis was performed using a TAQMAN® Gold RT Master Mix (Applied Biosystems, Foster City, Calif.) according to the manufacturer's protocol, except that both oligothymidylic (oligo[dT]) and random hexamers were used for priming. The quality of synthesized cDNA was assessed by generating expression profiles of endogenous reference genes with a real-time polymerase chain reaction (PCR) system, the ABI PRISM® 7900HT Sequence Detection System (Applied Biosystems, Foster City, Calif.). Complementary deoxyribonucleic acid samples were stored at −20° C.

Sequences for all biomarkers (Table 1) were derived from mouse and human deoxyribonucleic acid (DNA) sequence (GENBANK® database [online] maintained by National Center for Biotechnology Information, Bethesda, Md.). Quantitative reverse-transcriptase polymerase chain reaction assays were performed on an ABI PRISM® 7900HT Sequence Detection System (Applied Biosystems, Inc. (Foster City, Calif.) using a SYBR-green detection system (Qiagen Inc., Valencia, Calif.). All quantitative assays were run using universal thermal cycling parameters: hold at 95° C. for 2 minutes to activate the DNA polymerase, then run 40 three-part cycles (95° C. for 20 seconds, 58° C. for 20 seconds, and 76° C. for 20 seconds). Data were analyzed using sequence detection system (SDS) software, Version 1.7 (Applied Biosystems, Inc. (Foster City, Calif.). The first step was to generate an amplification plot for each sample, which showed the change in Rn (ΔRn) on the y-axis (where Rn is the fluorescence emission intensity of the reporter dye normalized to a passive reference) against the cycle number on the x axis. From each amplification plot, a threshold cycle (Ct) value was calculated. The Ct value is defined as the cycle at which a statistically significant increase in ΔRn is first detected and is displayed on the graph as the intercept point of the amplification plot and threshold. The Ct values were exported to an EXCEL® (MICROSOFT Corp., Redmond, Wash.) spreadsheet and relative expression (x) was calculated as shown in equation 2.

X=Power(2,−Ct)×100,000,000 EQUATION 2

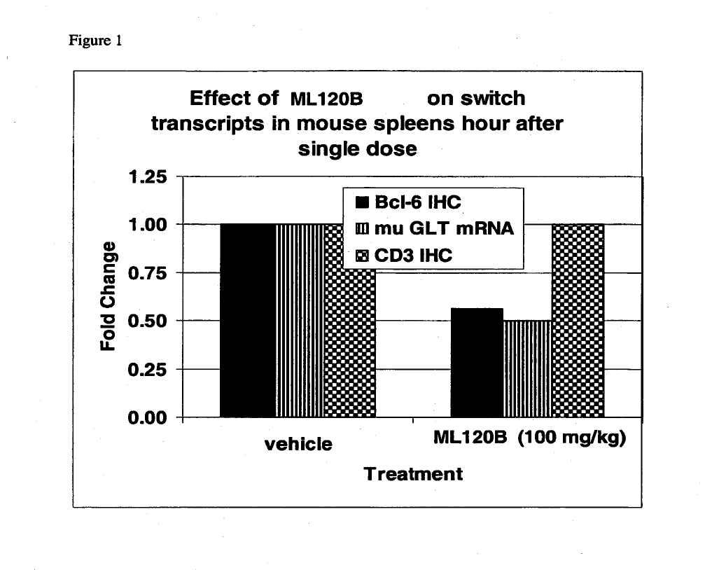

Mice were given a single oral dose of ML120B as indicated (n=4/group) for 6 hours. The level of mouse mu heavy chain germline transcript was measured in RNA isolated from serial cryosections by qRT-PCR. The frequency of germinal centers and the level of mu germline transcripts decreased by 50% within the first 6 hours of exposure to ML120B, whereas the frequency of T cells remained unchanged (FIG. 1).

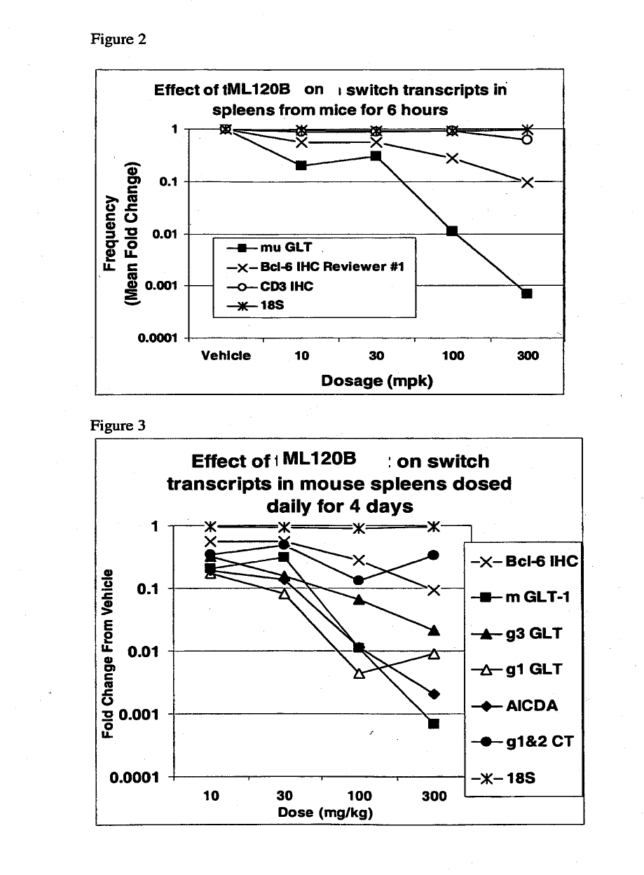

Mice were given a single oral dose of ML120B as indicated (n=4/group) for 18 hours. The level of mouse mu heavy chain germline transcript was measured in RNA isolated from serial cryosections by qRT-PCR. The frequency of germinal centers and the level of mu germline transcripts exhibited a dose-dependent decrease within the 18 hours of exposure to ML120B, with a 10-fold reduction in germinal centers and a 1000-fold reduction in mu germline transcripts occurring at 300 mg/kg (FIG. 2). The frequency of T cells and level of 18S rRNA, in contrast, remained unchanged (FIG. 2).

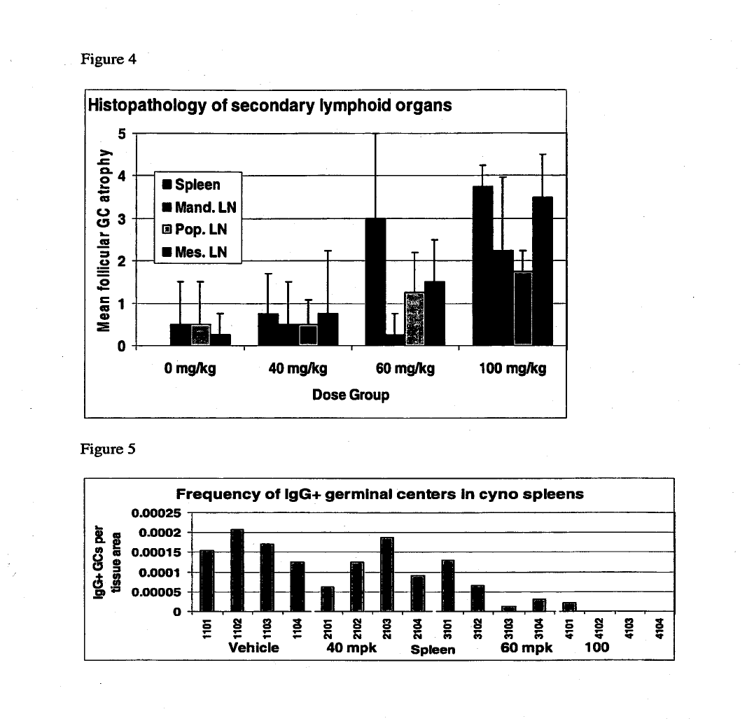

Mice were given twice daily oral dose of ML120B by gavage, as indicated (n=4/group) for four days. The level of mouse mu, gamma 3 and gamma 1 heavy chain germline transcript, AICDA transcript, gamma 1 & 2 circle transcript and 18S rRNA were measured in RNA isolated from serial cryosections by qRT-PCR. The frequency of germinal centers and the level of mu, gamma 3 and gamma 1 heavy chain germline transcript, AICDA transcript, gamma 1 & 2 circle transcript exhibited a dose-dependent decrease to ML120B (FIG. 3). Germinal centers expressing Bcl-6 decreased 10-fold at 300 mg/kg (FIG. 3). Germline transcripts mu, gamma 3 and gamma 1 decreased from 10-1000-fold at 300 mg/kg (FIG. 3). Transcripts encoding AICDA decreased 100-1000-fold at 300 mg/kg (FIG. 3). Circle transcript gamma 1&2 decreased 10-fold at 100 mg/kg (FIG. 3). The level of 18S rRNA, in contrast, remained unchanged (FIG. 3).

### Example 2

### Measurement of ICS in Monkeys

A proprietary immunomodulator, Test Agent A, was used as a test agent in these studies. The effect of Test Agent A on lymphocyte transcripts (CD19, CD20, GLT-μ, CT γ1&2, AID, TNF-α, IL-1β, AND 18S) was measured using a well-established, robust transcript assay, the quantitative reverse-transcription polymerase chain reaction (qRT-PCR), which demonstrates excellent sensitivity and specificity.

### Materials and Methods

Female cynomolgus monkeys (Macaca fascicularis, Charles River Laboratories (Sparks, Nev.; naive, nulliparous and non-pregnant, 2.0 to 4.0 kg) 4 animals per dosing group, 16 total). The monkeys were assigned to the study groups by weight-ordered distribution. Filtered tap water was available ad libitum.

The monkeys received Test Agent A, formulated in 0.1 M citrate buffer (pH 2.7±0.05), in 10 ml/kg daily for 28 days. Citrate buffer in water (0.1 M; PH 2.7±0.05) was the control used in this study. Both the Test Agent A and control articles were stored AT 5° C.±3° C. The animals received Test Agent A or control formulation once daily for 28 days (dosing phase). Doses were based on the most recently recorded body weight. Animals were administered by oral gavage a single volume of 10 ml/kg at doses of 0 (vehicle control), 40, 60, or 100 mg/kg. Following each dose, any residual control/Test Agent A remaining in the gavage tube was administered to the animal by administering 5 ml of tap water.

Once on Days −9, −2, 7, 14, 21, and 28, approximately 2.0 ml of peripheral blood from each animal was obtained via vein puncture and collected into PAXGENE® tubes (PREANALYTIX, Valencia, Calif.), following the manufacturer's instructions. Spleen specimens of all animals were flash frozen in liquid nitrogen at necropsy (Day 29). Blood samples and spleen specimens were stored at −70° C. until shipped on dry ice to Millennium Pharmaceuticals, Inc. (Cambridge, Mass.) and stored at −80° C.

### Biomarker Measurement

Ribonucleic Acid Isolation

Total ribonucleic acid (RNA) was isolated from 10 spleen cryosections, each 5 μM, using the RNEASY® Universal Tissue 8000 Kit (QIAGEN Inc., Valencia, Calif.) on a BIOROBOT® 8000 Workstation (QIAGEN INC., Valencia, Calif.), and deoxyribonucleic acidase (DNASE)-treated according to the manufacturer's protocol. Total RNA extraction from blood was conducted using the PAXGENE® 96 Blood RNA Kit (PREANALYTIX, Valencia, Calif.) according to the manufacturer's protocol. The purity and yield of the RNA were assessed via spectrophotometry from 220 to 750 nm using the NANODROP® ND-1000 (NANODROP Technologies, Wilmington, Del.). The integrity of the RNA was measured with the RNA 6000 NANO LABCHIP® Kit (AGILENT Technologies, Foster City, Calif.) on an AGILENT 2100 Bioanalyzer (AGILENT Technologies, Foster City, Calif.) and calculated using the RNA integrity number (RIN) algorithm (Schroeder et al, 2006). Deoxyribonucleic acidase-treated RNA was extracted from the samples and stored at −80° C. First strand complementary deoxyribonucleic acid (cDNA) synthesis was performed using a TAQMAN® Gold RT Master Mix (Applied Biosystems, Foster City, Calif.) according to the manufacturer's protocol, except that both oligothymidylic (oligo[dT]) and random hexamers were used for priming. The quality of synthesized cDNA was assessed by generating expression profiles of endogenous reference genes with a real-time polymerase chain reaction (PCR) system, the ABI PRISM® 7900HT Sequence Detection System (Applied Biosystems, Foster City, Calif.). Complementary deoxyribonucleic acid samples were stored at −20° C.

Quantitative Reverse-Transcriptase Polymerase Chain Reaction Reagent Design and Validation

Sequences for the reagents (Table 2) were derived from the human deoxyribonucleic acid (DNA) sequence (GENBANK® database [online] maintained by National Center for Biotechnology Information, Bethesda, Md.).

TABLE 2
Custom quantitative reverse-transcriptase polymerase chain reaction reagent
sequences

| | Reference | | | SEQ |
|---|---|---|---|---|
| | Sequence | | | ID |
| Biomarker | Identifier | Oligomer | Sequence | NO: |
| GLT-μ | NG_001019 | Forward | CCTGAATTA*TTTCAGTTAAGCATGT | 34 |
| | SEQ ID NO: | Reverse | CCTCGTCTCCTGTGAGAAT | 35 |
| | | Probe | CTCCATCA*CTTTCTCC | 36 |
| CT-γ1&2 | NG_001019 | Forward | CAACAGGGCAGGACAC | 37 |
| | SEQ ID NO: | Reverse | CCTCGTCTCCTGTGAGAAT | 35 |
| | | Probe | CTCCATCA*CTTTCTCC | 36 |
| IL-1β | NM_000576.2 | Forward | GAGGATCTCCTGTCCATCAG | 38 |
| | SEQ ID NO: | Reverse | CCAAATGTGGCCGTGGTTTCTGTCA | 39 |
| | | Probe | TCACCTCTCCTACTCACT | 40 |
| TNF-α | NM_000594 | Forward | CTAGAAATTGACACAAGTGGACCTT | 41 |
| | SEQ ID NO: | Reverse | CCCGGTCTCCCAAATAAATACATTC | 42 |
| | | Probe | GCCTTCCTCTCTCCA* | 43 |
| CD19 | NM_001770.4 | Forward | CTGTGACTTTGGCTTATCTGATCT | 44 |
| | SEQ ID NO: | Reverse | GCGTCACTTTGAAGAATCTCCTGGT | 45 |
| | | Probe | GCCTGTGTTCCCTTGTG | 46 |
| CD20 | NM_021950.3 | Forward | CCAATGAAAGGCCCTATTGCTAT | 47 |
| | SEQ ID NO: | Reverse | CAGTGAAGACATCCTCCTGAAGAGT | 48 |
| | | Probe | AATCTGGTCCA*AAAC | 49 |
| 18S | X03205.1 | Forward | G(A*)CACGG(A*)CAGG(A*)TTGACAGATTGATAG | 50 |
| | SEQ ID NO: | Reverse | GTT(A*)GCATGCCAGAGTCTCGTTCGTT | 51 |
| | | Probe | CCGTTCTT(A*)GTTGGTGG | 52 |
| AID | NM_020661.1 | AOD^{a} | ABI CATALOGUE NO. HS00757808_M1 | |
| | SEQ ID NO: | | | |
| IGL-κ | BC070336.1 | AOD^{a} | ABI CATALOGUE NO. HS00736177_M1 | |
| | SEQ ID NO: | | | |

GLT-μ, = Germline transcript mu; CT γ1&2 = circle transcripts containing gamma 1 and 2; IL-1β = Interleukin-1 beta; TNF-α = tumor necrosis factor alpha; IGL-κ = immunoglobulin light chain kappa; AOD = assay on demand. * = SUPER ™ Modified Base Technology from Nanogen (Bothell, WA). ^{a}: assays on demand from Applied Biosystems, Inc. (Foster City, CA).

Probes were MGB ECLIPSE™ Probes (NANOGEN, Bothell, Wash.). Each primer and probe pair were synthesized by NANOGEN (Bothell, Wash.) and were validated by measuring PCR efficiency using synthetic templates and human cDNA standards. Validated assays demonstrated linear amplification and >99% efficiency over 7 orders of magnitude (data not shown). Validated primer and probe pairs were sent to Millennium Pharmaceuticals, Inc. (Cambridge, Mass.) and were revalidated on the human positive control cDNA standard prior to use in expression profiling studies (data not shown). The IGL-x and AID assays used TAQMAN® reagents numbers Hs00736177_m1 and Hs00757808_m1, respectively (Applied Biosystems, Inc. (Foster City, Calif.).

Quantitative Reverse-Transcriptase Polymerase Chain Reaction Assay

Quantitative reverse-transcriptase polymerase chain reaction assays were performed on an ABI PRISM® 7900HT Sequence Detection System (Applied Biosystems, Inc. (Foster City, Calif.). All quantitative assays designed using NANOGEN (Bothell, Wash.) guidelines were run using universal thermal cycling parameters: hold at 95° C. for 2 minutes to activate the DNA polymerase, then run 40 three-part cycles (95° C. for 20 seconds, 58° C. for 20 seconds, and 76° C. for 20 seconds).

### Data Analysis

Raw Data Analysis

Data were analyzed using sequence detection system (SDS) software, Version 1.7 (Applied Biosystems, Inc. (Foster City, Calif.). The first step was to generate an amplification plot for each sample, which showed the change in Rn (ΔRn) on the y-axis (where Rn is the fluorescence emission intensity of the reporter dye normalized to a passive reference) against the cycle number on the x axis. From each amplification plot, a threshold cycle (Ct) value was calculated. The Ct value is defined as the cycle at which a statistically significant increase in ΔRn is first detected and is displayed on the graph as the intercept point of the amplification plot and threshold. The Ct values were exported to an EXCEL® (MICROSOFT Corp., Redmond, Wash.) spreadsheet and relative expression (x) was calculated as shown in Equation 2 (see Example 1).

Statistical Analyses

For each transcript examined in the blood, each animal's baseline was defined as the average of the Day −9 and Day −2 cycle numbers. The animal's baseline was subtracted from the animal's cycle number at each postdose time point. This difference was analyzed using a repeated measures linear regression model that included independent variables for the animal's baseline cycle number, dosage of the Test Agent A, time point (days postdose) at which the blood sample was taken, and the interaction between dose and time. In models where the dose-by-time interaction had a p-value greater than 0.05, the interaction term was removed from the model and the differences among doses were evaluated.

The model employed is shown in equation 3.

y_{itd}−yᵢₜ₀=yᵢₜ₀+Treatₜ+Day_{d}+(Treat*Day)_{td}+e_{itd} Equation 3.

where: y_{itd}=Cycle number for animal i at treatment t and day d; yᵢₜ₀=Cycle number for animal i and treatment t at baseline (d=0); Treatₛ=t level of treatment (vehicle, 40, 60 or 100 mg per kg of Test Agent); Day_{d}=d level of day (7, 14, 21, or 28 days); and (Treat*Day)_{td}=interaction between treatment and day (This tested the null hypothesis that there was no significant difference in the trend over time among the treatment groups); and =unexplained error incorporating correlation from repeated readings over time on same animal.

For each transcript examined in the spleen at Day 29, a Kruskal-Wallis test was performed to assess if there were any significant differences among any of the Test Agent A dose groups with respect to the transcript cycle number. All hypotheses were tested at a type I error rate of 0.05 (2-sided). Since this was an exploratory analysis, no adjustments for multiple comparisons were made to account for the number of transcripts and tissue types examined.

Histology

Hematoxylin and Eosin (H&E)-stained slides were prepared from spleen cryosections (5 μm) adjacent to those used for genomic analyses. Reactivity (hyperplasia) of spleen sections was assessed independently by 2 blinded reviewers, one of whom was a board-certified human anatomic pathologist. Reactivity was scored on a zero (minimal) to 4 (maximal) scale, based on measurements of the frequency and hyperplastic appearance of germinal centers in five 40× fields. These analyses exhibited a Pearson correlation coefficient value (r²) of 0.9, indicating that the analyses were consistent with each other.

### Results and Discussion

General Immunology Assessment

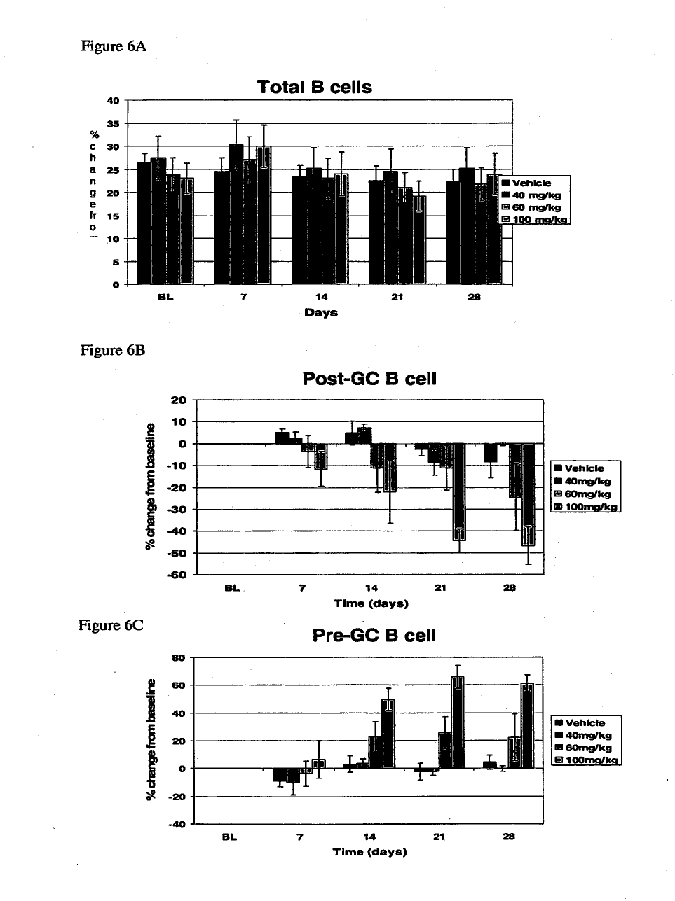

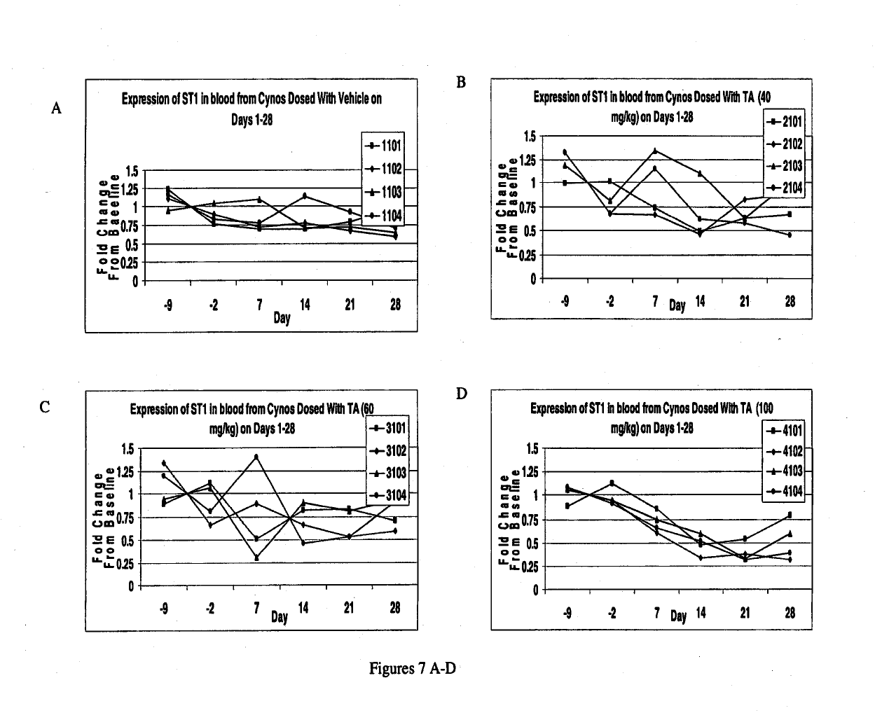

Histopathology of secondary lymphoid organs showed dose-dependent atrophy of GCs by the Test Agent A (see FIG. 4). Test Agent A decreases follicular dendritic cells and inhibits expansion of B cells. There was no change in frequency of B or T cells in secondary lymphoid organ. There was more than 100-fold decrease in proliferating B cells and more than 100-fold decrease in follicular dendritic cells. Test Agent A increases the relative frequency of immature B cells in secondary lymphoid organ as seen by immunofluorescence. Test Agent A decreases the frequency of mature B cells in the secondary lymphoid organ. Image analysis of immunoflourescence-stained IgG+ germinal centers in Cyno spleens showed a dose-dependent decrease in the frequency of IgG+ germinal centers (FIG. 5).

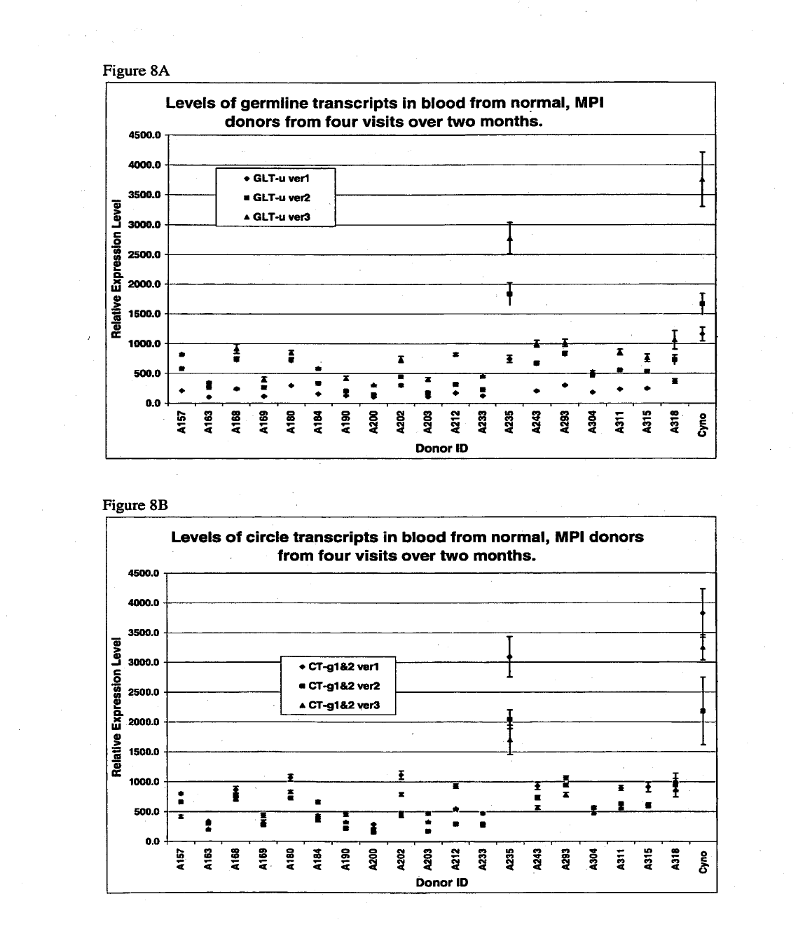

Test Agent A decreased the frequency of post-germinal center B cells in peripheral blood. As seen by flow cytometry of peripheral blood, there was no change in the frequency of total B cells. (FIG. 6A). However, there was a decrease in post-germinal center (GC) B cells (FIG. 6B) with a concomitant increase in pre-GC B cells (FIG. 6C).

Histologic Analysis of Spleen Cryosections

Analysis of H&E sections from the spleen of each animal by 2 independent, blinded reviewers confirmed that germinal center atrophy had been achieved in this study. (See FIG. 4) The vehicle control group (animal nos. 1101 through 1104) was completely homogenous, with each animal exhibiting highly reactive germinal centers and no indication of atrophy. The 40 mg/kg dose group (animal nos. 2101 through 2104) exhibited marked heterogeneity, with one animal exhibiting low germinal center reactivity, another exhibiting moderate reactivity, and 2 others exhibiting high reactivity. The 60 mg/kg dose group (animal nos. 3101 through 3104) was relatively homogeneous, with 3 animals exhibiting low reactivity and one exhibiting high reactivity. The 100 mg/kg dose group (animal nos. 4101 through 4104) was completely homogenous, with all 4 animals exhibiting low germinal center reactivity. A subset of animals in the 100 mg/kg group also exhibited symptoms of bacterial infection. The quantitative analyses of the 2 independent, blinded reviewers were consistent with one another (r²=0.9), which indicates the heterogeneity observed between subjects was not due to variability in the analyses, but to inherent differences in the tissues. Analysis of the data by dose illustrated a statistically significant (p<0.05) decrease in the reactivity of germinal centers in the 100 mg/kg dose group as compared to the vehicle control group.

Quantitative Reverse-Transcriptase Polymerase Chain Reaction Analysis of Spleen Cryosections

Isolated RNA from cryosections bordering the H&E sections (i.e., serial sections) was analyzed with qRT-PCR transcript assays to determine if the expression profiles of these transcripts (18S, TNF-α, IL-1β, CD20, CD19, IGL-κ, GLT-μ, AID, and CT-γ1&2) reflected germinal center atrophy. There was a dose-response trend in the spleen transcripts, as transcript numbers for all genes examined decreased with higher doses of Test Agent A, with the largest differences observed for AID and TNF-α (Table 3) illustrates the values for the control (vehicle) animals and the 100 mg/kg-treated animals).

TABLE 3
Expression results after 28 days of treatment with Test Agent A

| | Vehicle | | | 100 mg/kg | | |
|---|---|---|---|---|---|---|
| Biomarker | Expression | St. Dev | T-test | Expression | St. Dev | T-test |
| CT gamma | 20.99 | 11.39 | 1 | 3.84 | 2.08 | 0.025 |
| 1 and 2 | | | | | | |
| GLT-mu | 17.06 | 9.79 | 1 | 4.12 | 2.11 | 0.042 |
| AID | 0.0234 | 0.015 | 1 | 0.00048 | 0.00035 | 0.023 |
| TNF-alpha | 0.0986 | 0.072 | 1 | 0.0063 | 0.0037 | 0.044 |
| IL-1beta | 0.192 | 0.081 | 1 | 0.0398 | 0.030 | 0.013 |
| CD19 | 0.045 | 0.0091 | 1 | 0.0179 | 0.0088 | 0.0050 |
| CD20 | 14.8 | 5.73 | 1 | 2.75 | 1.46 | 0.0064 |
| IgL-kappa | 46.6 | 49.0 | 1 | 3.44 | 2.56 | 0.13 |
| 18S RNA | 23252 | 4759 | 1 | 14343 | 3569 | 0.024 |

With the exception of CD19, Kruskal-Wallis analyses of all transcripts showed significant (p<0.05) differences among the treatment groups (Table 4).

TABLE 4
Kruskal-Wallis results for transcript levels in the spleen

| | |
|---|---|
| Kruskal-Wallis Test | Median Ct^{b} |

| Protein | Overall p-Value^{a} | 0 mg/kg | 100 mg/kg |
|---|---|---|---|
| 18S | 0.0060 | 12.03 | 12.73 |
| TNF-α | 0.0037 | 29.47 | 34.37 |
| IL-1β | 0.0086 | 28.86 | 31.71 |
| CD20 | 0.0151 | 22.55 | 25.09 |
| CD19 | 0.0905 | 31.18 | 32.30 |
| IGL-κ | 0.0415 | 20.79 | 24.93 |
| GLT-μ | 0.0086 | 22.56 | 24.29 |
| AID | 0.0143 | 32.18 | 38.78 |
| CT-γ1&2 | 0.0068 | 22.70 | 24.83 |

^{a}Overall p-value tested the hypothesis that there were no differences among treatment group medians.
^{b}Median data for 0 and 100 mg/kg dose groups included to illustrate significant differences

A large portion of the significance observed for each gene was attributed to the differences between the vehicle control group and the 100 mg/kg dose group (Table 4). These analyses also suggested a significant dose-dependent effect on 18S, the transcript used as an internal reference. This effect was small (<2-fold effect) and it is unknown whether it represented a biological effect or was a technical artifact. These data were nonetheless reanalyzed after normalizing the data for differences in 18S values, an endogenous reference transcript, and the conclusions remained unchanged (Table 5). The expression profiles of these transcripts correlated positively with histologic metrics of reactivity.

TABLE 5
Kruskal-Wallis results for transcript levels
in spleen (data normalized to 18S)

| | |
|---|---|
| Kruskal-Wallis Test | Median delta Ct^{b} |

| Protein | Overall p-Value^{a} | 0 mg/kg | 100 mg/kg |
|---|---|---|---|
| 18S | — | — | — |
| TNF-α | 0.0041 | 17.32 | 21.70 |
| IL-1β | 0.0257 | 16.98 | 18.98 |
| CD20 | 0.0496 | 10.69 | 12.46 |
| CD19 | 0.3314 | 19.03 | 19.67 |
| IGL-κ | 0.0506 | 8.77 | 12.30 |
| GLT-μ | 0.0127 | 10.49 | 11.56 |
| AID | 0.0136 | 20.24 | 25.81 |
| CT-γ1&2 | 0.0083 | 10.63 | 12.10 |

^{a}Overall p-value tested the hypothesis that there were no differences among treatment group medians.
^{b}Data were normalized to a conventional internal reference transcript, 18S (assumes that 18S is not regulated by Test Agent A). Median delta Ct = median Ct value of Protein X − Median Ct value of 18S for a given sample. Median data for 0 and 100 mg/kg dose groups included to illustrate significant differences.

The Test Agent A exhibited a slight dose-dependent effect on one immunohistologic marker of B cells, CD20, but not another, CD19, in the spleen. The effect on CD20 was small and, while statistically significant, was not reproduced in peripheral blood samples; the Test Agent A did not significantly affect CD20 levels in peripheral blood. The overall cellularity of the spleen (e.g., organ weights, histology) did not change in a dose-dependent manner. Additional immunohistochemistry analyses that focused on the content of B cells within cryosections from spleen samples indicated that the frequency of B cells expressing CD20 protein did not differ between dose groups (data not shown). These additional data suggest that the small differences observed in CD20 transcripts in the spleen are not systemic effects and are unlikely to be of biological significance.

Quantitative Reverse-Transcriptase Polymerase Chain Reaction Analysis of Peripheral Blood

Changes in the levels of IGL-κ, GLT-μ, AID, and CT-γ1&2 transcripts in peripheral blood were also significantly-dose-dependent, with transcript levels decreasing with higher doses of the Test Agent A (Table 6; treatment p-value).

TABLE 6
Kruskal-Wallis results for transcript levels in blood

| | Time-by-Treatment | |
|---|---|---|
| Protein | Interaction p-Value^{a} | Treatment p-Value^{a} |
| 18S | NS | 0.3064 |
| TNF-α | NS | 0.2499 |
| IL-1β | NS | 0.5528 |
| CD20 | NS | 0.0954 |
| CD19 | NS | 0.2820 |
| IGL-κ | NS | 0.0007 |
| GLT-μ | NS | 0.0051 |
| AID | NS | 0.0335 |
| CT-γ1&2 | NS | 0.0212 |

NS = not significant.
^{a}Overall p-value tested the hypothesis that there were no differences among treatment group medians. Dunn's Post Test compared each treated group to the vehicle.

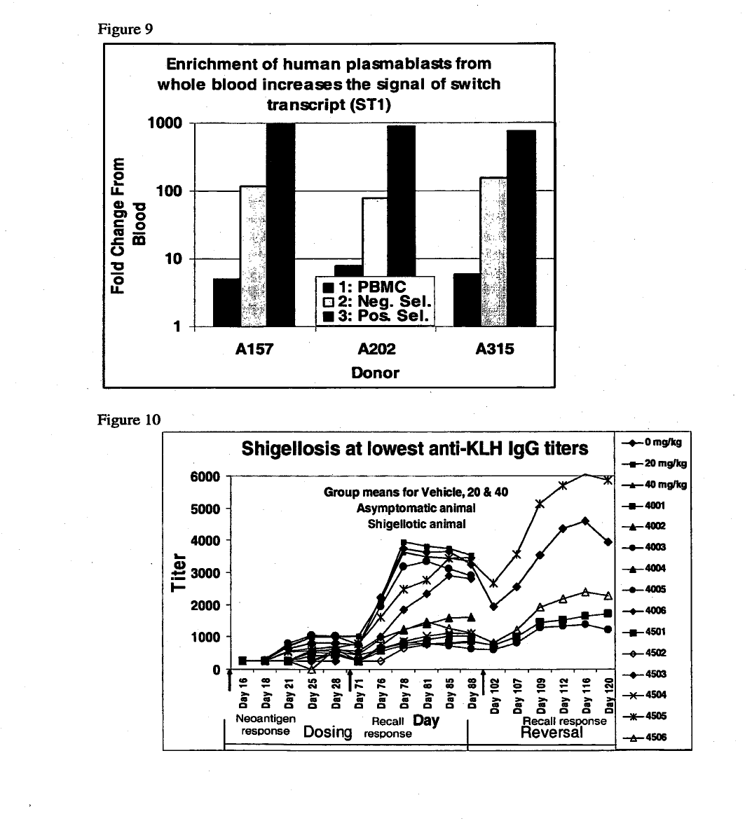

A large portion of the significance observed for each gene was attributed to the differences between the vehicle control group and the 100 mg/kg dose group. The other Test Agent A-treated groups did not differ greatly from the vehicle control group. There was considerable variability among animals within treatment groups (FIG. 7 A-D illustrate results for GLT-mu (ST1)). The repeated measures linear model indicated that there were no significant differences in the trends over time among the treatment groups for any of the transcripts (Table 6, time-by-treatment interaction p-value).

Comparison of Histology and Quantitative Reverse-Transcriptase Polymerase Chain Reaction Data

Comparisons of histology and spleen qRT-PCR data demonstrated that levels of IGL-κ, GLT-μ, AID, and CT-γ1&2 transcripts are predictive of germinal center reactivity. Analyses of data from individual animals highlighted positive correlations between histologic germinal center reactivity data and levels of these transcripts. These positive correlations were not evident in analyses of the data by dose group, particularly in the 40 and 60 mg/kg dose groups, where interanimal variability was greatest. Histology indicated that animal nos. 1101, 1102, 1103, 1104, 2102, 2103, and 3101 exhibited high germinal center reactivity, while animal nos. 2101, 2104, 3102, 3103, 3104, 4101, 4102, 4103, and 4104 exhibited low germinal center reactivity. Analyses of data for all transcripts, from all spleens revealed that IGL-κ, GLT-μ, AID, and CT-γ1&2 exhibited a similar pattern. The No Observable Effect Level (NOEL) was determined to be 60 mg/kg, on the basis of the changes in these novel transcript biomarkers, which are considered nonadverse. This NOEL agrees with that determined by histologic assays. Infection was considered an adverse effect that defined the No Observable Adverse Effect Level (NOAEL) in previous investigations. Infections were not observed in this investigation.

More importantly, levels of IGL-κ, GLT-μ, AID, CT-γ1&2, CD19, and CD20 transcripts in peripheral blood exhibited the highest correlations with germinal center reactivity data from histology (Table 7).

TABLE 7
Comparison (r²) of slopes of blood transcript
levels to histology reactivity scores

| Blood Transcript | R² Value |
|---|---|
| IGL-κ | 0.65 |
| GLT-μ, | 0.42 |
| AID | 0.56 |
| CT-γ1&2 | 0.54 |
| CD19 | 0.63 |
| CD20 | 0.41 |
| TNF-α | 0.01 |
| IL-1β | 0.01 |
| 18S | 0.13 |

These correlations are most evident from analyses of individual animals and not by dose group. Transcript levels of IGL-κ, GLT-μ, AID, and CT-γ1&2 from the blood of individual animals were examined within dose groups. The linear slope of transcript level versus time was calculated, in order to control for differences in the magnitude of expression between animals and for variability in measurements at a specific time point. A drug effect could manifest itself as a decrease in transcript levels with time, yielding a negative slope value, the magnitude of which would be proportional to the magnitude of the decrease. If there were no drug effect, there would not be a change in transcript levels with time, yielding a relatively flat slope with a value approaching zero. The calculated slopes correlated with qRT-PCR and histology data from the spleens of individual animals. Animal no. 2101, for example, exhibited low germinal center reactivity values as determined by histology, splenic qRT-PCR, and blood qRT-PCR, and these values were comparable to those in the 100 mg/kg dose group (animal nos. 4101, 4102, 4103, and 4104). The magnitude of the decrease of blood switch transcripts over time (i.e. slope of days −2 to 28) predicts animals with normal versus atrophic germinal centers on day 29.

Animals were classified as normal or abnormal (i.e., potentially atrophic), with respect to the reactivity of their germinal centers, based on the slope values of the blood transcript levels. Peripheral blood biomarkers which are only expressed by B lymphocytes (IGL-κ, GLT-μ, AID, and CT-γ1&2), exhibited the strongest associations with the histology data (Table 8).

TABLE 8
Potential accuracy of blood transcript biomarkers
for predicting splenic germinal center atrophy

| | | Potential Accuracy at |
|---|---|---|
| Blood Transcript | Classification | Predicting Splenic Atrophy |
| IGL-κ | B cell-specific | 100% |
| GLT-μ, | B cell-specific | 88% |
| AID | B cell-specific | 88% |
| CT-γ1&2 | B cell-specific | 88% |
| CD19 | B cell-specific | 75% |
| CD20 | B cell-specific | 75% |
| TNF-α | ubiquitous | 62% |
| IL-1β | ubiquitous | 62% |
| 18S | ubiquitous | 50% |

The slope values of the blood IGL-K transcripts exhibited the highest predictive potential, as they correctly designated all subjects as exhibiting normal or abnormal splenic germinal center reactivity (Table 9). The slope values of the blood GLT-μ, AID, and CT-γ1&2 transcripts were less correlative and somewhat less predictive, each classifying 88% of the subjects correctly (Table 10, Table 11, and Table 12 respectively).

TABLE 9
Use of the slope of blood IGL-κ levels over time at predicting
reactivity (e.g., atrophy) of germinal centers in spleen

| | Animal | Spleen Histology |
|---|---|---|
| Blood IGL-κ Slope | No. | Assessment |
| y = 0.0037x | 1104 | Normal |
| y = 0.0043x | 2103 | Normal |
| y = 0.007x | 2102 | Normal |
| y = −0.0156x | 1102 | Normal |
| y = −0.0342x | 1103 | Normal |
| y = 0.038x | 3101 | Normal |
| y = 0.061x | 1101 | Normal |
| y = −0.0666x | 3102 | Abnormal |
| y = −0.1003x | 2104 | Abnormal |
| y = −0.1102x | 4101 | Abnormal |
| y = −0.119x | 2101 | Abnormal |
| y = −0.1295x | 3104 | Abnormal |
| y = −0.2419x | 4104 | Abnormal |
| y = −0.2911x | 3103 | Abnormal |
| y = −0.2911x | 4102 | Abnormal |
| y = −0.3127x | 4103 | Abnormal |

TABLE 10
Use of the slope of blood GLT-μ levels over time at predicting
reactivity (e.g., atrophy) of germinal centers in spleen

| | Animal | Spleen Histology |
|---|---|---|
| Blood GLT-μ Slope | No. | Assessment |
| y = −0.0101x | 3101 | Normal |
| y = 0.0375x | 1101 | Normal |
| y = −0.0377x | 2102 | Normal |
| y = −0.0572x | 2103 | Normal |
| y = −0.0649x | 3104 | Abnormal |
| y = −0.0731x | 1104 | Normal |
| y = −0.0829x | 1103 | Normal |
| y = −0.099x | 2101 | Abnormal |
| y = −0.1007x | 4101 | Abnormal |
| y = −0.1093x | 4103 | Abnormal |
| y = −0.1214x | 1102 | Normal |
| y = −0.1558x | 3102 | Abnormal |
| y = −0.1692x | 2104 | Abnormal |
| y = −0.231x | 4104 | Abnormal |
| y = −0.2364x | 3103 | Abnormal |
| y = −0.2364x | 4102 | Abnormal |

TABLE 11
Use of the slope of blood AID levels over time at predicting
reactivity (e.g., atrophy) of germinal centers in spleen

| | Animal | Spleen Histology |
|---|---|---|
| Blood AID Slope | No. | Assessment |
| y = 0.0127x | 2102 | Normal |
| y = 0.0254x | 3101 | Normal |
| y = −0.0798x | 1104 | Normal |
| y = −0.07x | 2103 | Normal |
| y = −0.0908x | 1103 | Normal |
| y = 0.1081x | 1101 | Normal |
| y = −0.1267x | 3104 | Abnormal |
| y = −0.1304x | 1102 | Normal |
| y = −0.154x | 4101 | Abnormal |
| y = −0.1681x | 2101 | Abnormal |
| y = −0.1701x | 2104 | Abnormal |
| y = −0.1824x | 3102 | Abnormal |
| y = −0.2741x | 4103 | Abnormal |
| y = −0.2755x | 3103 | Abnormal |
| y = −0.2755x | 4102 | Abnormal |
| y = −0.3143x | 4104 | Abnormal |

TABLE 12
Use of the slope of blood CT-γ1&2 levels over time at predicting
reactivity (e.g., atrophy) of germinal centers in spleen

| | Animal | Spleen Histology |
|---|---|---|
| Blood CT-γ1&2 Slope | No. | Assessment |
| y = −0.0385x | 1101 | Normal |
| y = −0.0473x | 2103 | Normal |
| y = −0.0507x | 1104 | Normal |
| y = −0.0519x | 3101 | Normal |
| y = −0.0698x | 2102 | Normal |
| y = −0.0953x | 4101 | Abnormal |
| y = −0.0958x | 1103 | Normal |
| y = −0.1037x | 3104 | Abnormal |
| y = −0.1138x | 2101 | Abnormal |
| y = −0.1156x | 1102 | Normal |
| y = −0.1593x | 3102 | Abnormal |
| y = −0.1737x | 4103 | Abnormal |
| y = −0.1852x | 2104 | Abnormal |
| y = −0.2397x | 4104 | Abnormal |
| y = −0.2685x | 3103 | Abnormal |
| y = −0.2685x | 4102 | Abnormal |

The next most predictive subcategory of biomarkers included those which are only expressed by B lymphocytes (CD19 and CD20). CD19 and CD20 were less predictive than IGL-κ, GLT-μ, AID, and CT-γ1&2, each having correctly classified 75% of the animals (Table 8).

A third biomarker subcategory includes those whose expression is associated with NF-κB activity and which are ubiquitously expressed (TNF-α and IL-1β). These two transcripts were less predictive than CD19 and CD20, each having correctly classified 62% of the animals (Table 8).

The last biomarker studied, 18S, a non-mechanistic and ubiquitous biomarker, was not expected to be predictive. It correctly classified 50% of the animals as normal or abnormal, which is no better than random chance (Table 8).

Conclusion

Several biomarker transcripts (IGL-κ, GLT-μ, AID, and CT-γ1&2) were identified in peripheral blood to be predictive of splenic germinal center atrophy caused by exaggerated doses of Test Agent A within the context of this study. The NOEL was determined to be 60 mg/kg on the basis of changes in these novel transcript biomarkers, which are considered nonadverse. This NOEL agrees with that determined by histologic assays. Infection is considered an adverse effect that defined the noael in previous investigations. Infections were not observed in this investigation.

### Example 3

### Measurement of ICS in Peripheral Human Blood

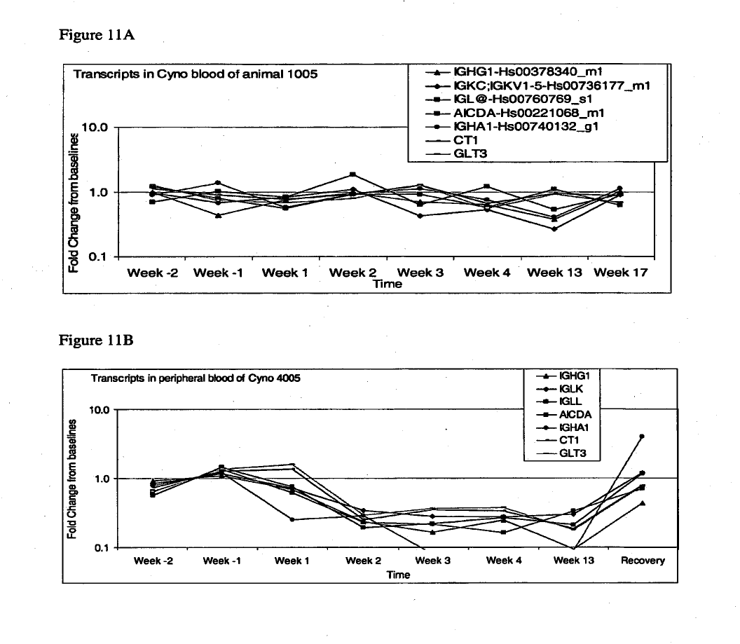

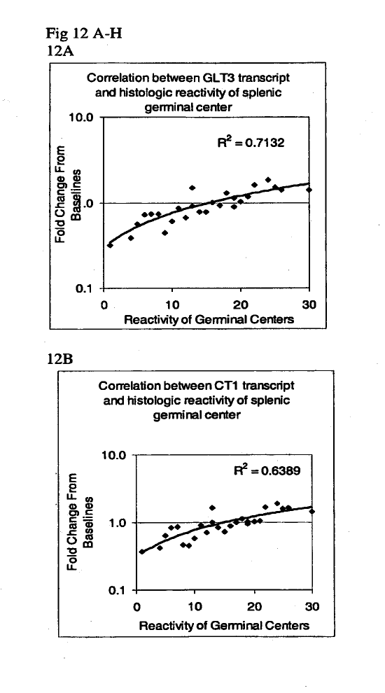

Peripheral blood was collected from healthy human volunteers and utilized as a surrogate tissue to detect splenic germinal center atrophy. Samples were collected from 19 donors at 4 timepoints each (two weeks apart), the RNA was isolated as described for the monkey assays and the expression of ICS markers, e.g., GLT-μ and circle transcripts CT-γ1&2 (using GLT1, GLT2, GLT3, CT1, CT2, and CT3), was determined. The detection reagents were designed to detect consensus sequences between human and monkey (see Table 13 for sequences). All the markers were detected in peripheral blood (FIGS. 8A and B). A relative expression level shows a general low level of transcripts. Since the volunteers generally were healthy, there was no expectation of differences between timepoints or between volunteers. Therefore, in order to show that differences could be detected, the samples were modified to increase the likelihood of detecting switch transcripts. Plasmablasts were enriched in the samples by negative selection of spinning through a polysaccharide cushion, then assayed or further selected by positive selection using magnetic beads conjugated to a CD138-detecting reagent (Miltenyi Biotec, Auburn, Calif.). The result is shown in FIG. 9. The level of ICS transcript (circle transcript CT-γ1&2) is proportional to the frequency of human plasmablasts in whole blood sample.

### Example 4

### Measurement of ICS in Monkey

Examples 1-3 demonstrated that switch transcripts could be detected and changes in their levels could be measured in peripheral blood of mice, cynomolgous macaques and normal human subjects. Example 2 also demonstrated that levels of switch transcripts in peripheral blood reflected the degree of follicular germinal center atrophy in spleens of cynomolgous macaques. The goals of the current study were to 1) validate these previous observations in the context of a 13-week study. 2) assess if levels of switch transcripts in peripheral blood could also serve as antecedent markers of clinical symptoms of bacterial infection.

Both male and female cynomolgus monkeys (Macaca fascicularis, Charles River Laboratories (Sparks, Nev.; naive, nulliparous and non-pregnant, 2.0 TO 4.0 kg, 40 total). The monkeys were assigned to the study groups by weight-ordered distribution. Filtered tap water was available ad libitum.

The monkeys received Test Agent A, formulated, stored and administered as in Example 2, or control once daily for 13 weeks (91 days). Doses were 0, 20, 40, or 80 mg/kg.

Peripheral blood samples were collected using the same procedure as in Example 2. Samples were obtained prior to initiation of dosing (Weeks −2 and −1) and during Weeks 1, 2, 3, 4, 13, and 17 (end of recovery period). Blood samples were stored at −70° C. until shipped on dry ice to Millennium Pharmaceuticals, Inc. (Millennium, Cambridge, Mass.) and stored at −80° C.

Spleen biopsy specimens were collected from all euthanized animals. Thirty-two animals (4/sex/group) were euthanized one day after the last dose (Day 92) and 8 animals (four controls and four 80 mg/kg animals, 2/sex/group) were euthanized 4 weeks after the last dose (recovery group, Day 121 or 122). Necropsy was performed on all euthanized animals and spleen biopsy specimens were preserved in neutral-buffered 10% formalin.

RNA Isolation

Total RNA extraction from the blood samples, RNA QC and cDNA generation was performed at Asuragen Inc. (Austin Tex.), using the general steps similar to those used in Example 2. cDNA samples were stored at −20° C.

Quantitative Reverse-Transcriptase Polymerase Chain Reaction

Sequences for all biomarkers were derived from the human deoxyribonucleic acid (DNA) sequence (GenBank® nucleic acid database, National Center for Biotechnology Information, Bethesda, Md.) Primers and MGB Eclipse™ probes for GLT-μ and CT1 (Nanogen, Bothell, Wash.) were designed from analysis. Each primer and probe pair was synthesized by Nanogen and was validated by measuring PCR efficiency using synthetic templates and human cDNA standards. Validated assays demonstrated linear amplification and >99% efficiency over 7 orders of magnitude (data not shown). Validated primer and probe pairs were sent to Millennium (Cambridge, Mass.) and were revalidated on the human positive control cDNA standard prior to use in expression profiling experiments. The Applied Biosystem Assay on Demand (AOD) assays found in Table 13 were configured into the Millennium DSD718 TaqMan® Low Density Array (Applied Biosystems; Foster City, Calif.).

TABLE 13
Biomarker reagents

| | Reference | | | SEQ |
|---|---|---|---|---|
| | Sequence | | | ID |
| Biomarker | Identifier | Oligomer | Sequence | NO: |
| GLT-μ ver1 | NG_001019 | Forward | ACAGTCTTAGGGAGAGITTATGAC | 53 |
| (GLT1) | | Reverse | CACCACGTGTTCGTCTGTG | 54 |
| | | Probe | CTCCATCA*CTTTCTCC | 36 |
| GLT-μ ver2 | NG_001019 | Forward | GATATTCTGATAGAGTGGCCTTCAT | 55 |
| (GLT2) | | Reverse | CACCACGTGTTCGTCTGTG | 54 |
| | | Probe | CTCCATCA*CTTTCTCC | 36 |
| GLT-μ ver3 | NG_001019 | Forward | CCTGAATTA*TTTCAGTTAAGCATGT | 34 |
| (GLT3) | | Reverse | CACCACGTGTTCGTCTGTG | 54 |
| | | Probe | CTCCATCA*CTTTCTCC | 36 |
| CT-γ1&2 ver1 | NG_001019 | Forward | CAACAGGGCAGGACAC | 37 |
| (CT I) | | Reverse | CACCACGTGTTCGTCTGTG | 54 |
| | | Probe | CTCCATCA*CTTTCTCC | 36 |
| CT-γ1&2 ver2 | NG_001019 | Forward | AGCAGAGCTGGCCGTA | 56 |
| (CT2) | | Reverse | CACCACGTGTTCGTCTGTG | 54 |
| | | Probe | CTCCATCA*CTTTCTCC | 36 |
| CT-γ1&2 ver3 | NG_001019 | Forward | CCAGAAAGGCCCAGAGT | 57 |
| (CT3) | | Reverse | CACCACGTGTTCGTCTGTG | 54 |
| | | Probe | CTCCATCA*CTTTCTCC | 36 |
| 18S | X03205.1 | AOD^{a} | ABI Catalog No. Hs99999901_s1 | |
| IGHD | | AOD^{a} | ABI Catalog No. Hs00378878_m1 | |
| IGKC | | AOD^{a} | ABI Catalog No. Hs00736177_m1 | |
| IGLL | | AOD^{a} | ABI Catalog No. Hs00760769_s1 | |
| AICDA | NM_020661 | AOD^{a} | ABI catalog No. Hs002210688_m1 | |
| IGHA | | AOD^{a} | ABI catalog No. Hs00740132_g1 | |
| CFB | | AOD^{a} | ABI Catalog No. Hs00175252_m1 | |
| B2MG | | AOD^{a} | ABI Catalog No. Hs99999907_m1 | |
| IGHG1 | | AOD^{a} | ABI Catalog No. Hs00378340_m1 | |

Quantitative reverse-transcriptase polymerase chain reaction assays were performed on an ABI PRISM® 7900HT Sequence Detection System (Applied Biosystems, Foster City, Calif.). All quantitative assays designed using Nanogen guidelines were run using the following universal thermal cycling parameters: hold at 95° C. for 2 minutes to activate the DNA polymerase, then run 40 three-part cycles (95° C. for 20 seconds, 58° C. for 20 seconds, and 76° C. for 20 seconds). All quantitative assays for the TAQMAN® Low Density Array were run using the following cycling parameters: hold at 50° C. for 2 minutes for AMPERASE® UNG (PCR Master Mix, Applied Biosystems, Foster City, Calif.) activation, then at 94.5° C. for 10 minutes to activate DNA polymerase, then run 40 two-part cycles (97° C. for 30 seconds and 59.7° C. for 1 minute).

Raw Data Analysis

Data were analyzed using Sequence Detection System (SDS) software, Version 2.2 (Applied Biosystems, Foster City, Calif.). The first step was to generate an amplification plot for each sample, which showed the change in Rn (ΔRn) on the y axis (where Rn is the fluorescence emission intensity of the reporter dye normalized to a passive reference) against the cycle number on the x axis. From each amplification plot, a threshold cycle (Ct) value was calculated. The Ct value is defined as the cycle number at which a statistically significant increase in ΔRn above background levels (i.e. noise) is detected. Ct values are displayed on a graph as the intercept point of the amplification plot and threshold. The Ct values were exported to an EXCEL® (Microsoft Corp., Redmond, Wash.) spreadsheet. Interpretation of raw Ct values can be confounded by variability in the amount of sample that was input into each assay. A conventional method of normalizing for such technical variability is to subtract the raw Ct value of an internal, invariant, endogenous reference transcript (18S or B2MG) from the raw Ct value of the transcripts of interest (in this case CT1, GLT3, IGLL, IGLK, AICDA, IGHG1, or IGHA1) to generate the delta cycle threshold (dCt) value. Transcripts for 18S rRNA (18S) or β-2 microglobulin (B2MG) are conventional invariant endogenous reference transcripts. Analysis of the data in this investigation illustrate that the 18S is the most invariant of the endogenous reference transcripts evaluated in this investigation. Cycle threshold and dCt are log base 2 (log₂) functions; an increase of one Ct represents a doubling of signal, whereas a decrease of one Ct represents a halving of signal. Levels of transcript are therefore represented as Normalized Level using Equation 3: [X=Power(2,−dCt)×1000].

The majority of transcripts examined in this investigation (GLT3, CT1, AICDA, IGHG1 and IGHA1) are likely to be co-regulated because they are products of germinal center reactions. Trend analysis of changes in any single transcript in the context of other co-regulated transcripts allows the determination of whether a change in any single transcript represents a nonspecific trend in all the transcripts or is limited to a specific subset of transcripts. However, the normalized level of different transcripts may vary by 100,000-fold, which impedes direct comparative analyses. The data was normalized for variability in the magnitude of signal by calculation of fold changes from baseline, thus simplifying comparison across transcripts. Fold change from baseline was calculated by, dividing the normalized value of a transcript at a given time-point by the mean of the two baseline normalized level values (Week −2 and Week −1).

### Results

General Immune Function

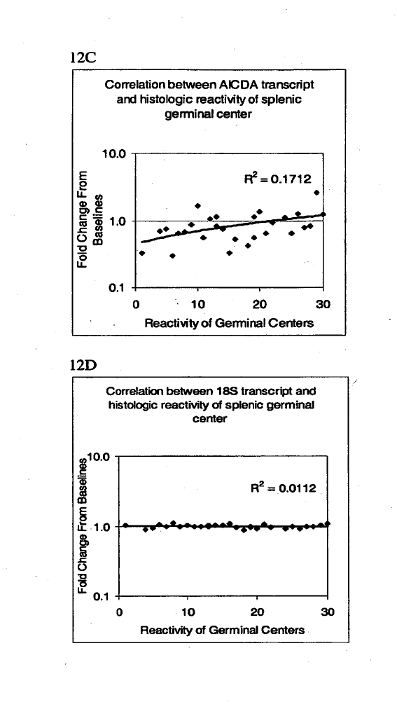

There were no treatment-related on clinical observations, except bacterial infection in a subset of high dose animals. Clinical pathology otherwise was normal. The anatomic pathology was normal, except there was atrophy of lymphoid germinal centers. An immunotoxicology assesment showed no inhibition of complement pathways; no inhibition of phagocytic function or respiratory burst; no inhibition of natural killer (NK) function; no alterations in frequency of major subsets of blood leukocytes; no change in levels of serum immunoglobulins; but inhibition of T cell-dependent antibody responses (TDAR) to keyhole limpet hemocyanin (KLH) (FIG. 10) in high dose animals. The impaired TDAR is associated with clinical symptoms of infection (FIG. 10); dose-dependent inhibition of the IgG response to neo- and recall antigen. Symptomatic animals exhibited the lowest anti-KLH titers, but overall Ig levels were unchanged.

Histologic Analysis

Analysis of H&E sections from the spleen of each animal by 2 independent, blinded reviewers confirmed that atrophy of germinal centers occurred in this investigation in a dose-dependent manner. Minimal atrophy was observed in two animals of the 40 mg/kg group and one animal of the 80 mg/kg group. Mild atrophy was observed in one animal of the 40 mg/kg group and one animal of the 80 mg/kg group. Moderate atrophy was observed in two animals of the 80 mg/kg group. Significant differences were not observed between dose groups due to high inter-animal variability within the 40 and 80 mg/kg groups. Reactivity of each animal euthanized by Week 13 was also scored on a relative scale, essentially ranking each specimen from 1 (least reactive) to 30 (most reactive) based on assessment of the frequency and magnitude of hyperplasia of germinal centers. The relative scale facilitated comparisons of histologic changes to levels of transcripts in peripheral blood. The overall degree of germinal center atrophy achieved in this investigation is less than that achieved in the previous 28-day investigation (Example 2), which may have resulted from administering lower doses (20, 40 and 80 mg/kg compared to 40, 60 and 100 mg/kg) and/or attaining lower exposures in the 13 week study compared to the previous 28-day study.

Quantitative RT-PCR

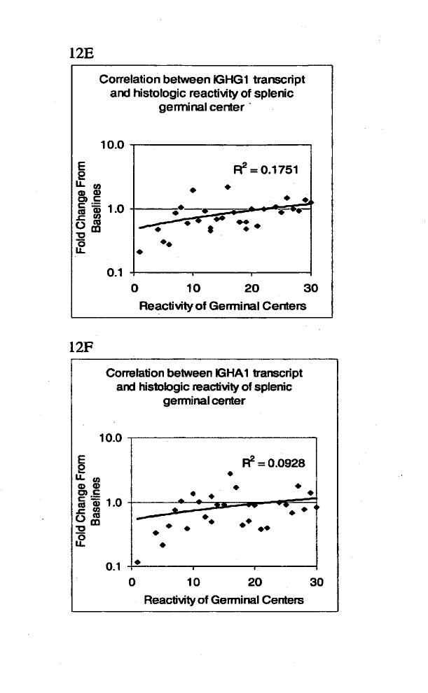

Significant differences were not observed for any transcript between dose groups. Levels of GLT3, CT1, AICDA, IGHA1, IGDG1, IGLK and IGLL transcripts exhibited coordinated variation over time within individual animals in the vehicle control group (see FIG. 11A, for example). This variation most likely represents biological (not technical) variation in levels of these transcripts over time because the GLT3 and CT1 assays were conducted with a different technology (i.e. MGB Eclipse probes) and format (384-well plates) than the AICDA, IGHA1, IGDG1, IGLK and IGLL assays (ie, TaqMan probes and TLDA microfluidic cards.

An effect of Test Agent A on the levels of IGHA1, IGDG1, GLT3, AICDA, CT1, IGLK or IGLL transcripts was not observed for individual animals in the 20 mg/kg group.

An effect of Test Agent A on the levels of IGHA1, IGDG1, GLT3, AICDA, CT1, IGLK or IGLL transcripts was generally not observed for individual animals within the 40 mg/kg group, although some may have occurred in animals 3001, 3003 and 3501during Weeks 3-13.

Changes in the levels of IGHA1, IGDG1, GLT3, AICDA, CT1, IGLK and IGLL transcripts were observed in response to Test Agent A (80 mg/kg) for animals 4004, 4005 and 4006 (see FIG. 11B, for example). Dosing of animals 4004 and 4006 was terminated during Week 4 due to the moribund condition of these animals. Animal 4005 was not moribund and dosing was continued, as this animal's condition consistently improved with administration of antibiotics with a return to normal clinical status by Week 17. The effects of Test Agent A on levels of IGHA1, IGDG1, GLT3, AICDA, CT1, IGLK and IGLL transcripts observed at Week 13 in animal 4005 were reversible, in that levels of these transcripts returned to their baseline levels by Week 17, four weeks after last dose of Test Agent A. There were no differences between the two control endogenous reference transcripts (18S and B2MG) between dose groups, nor changes in response to Test Agent A.

Comparison of Atrophy of Splenic Germinal Centers to Levels of Transcripts in Peripheral Blood

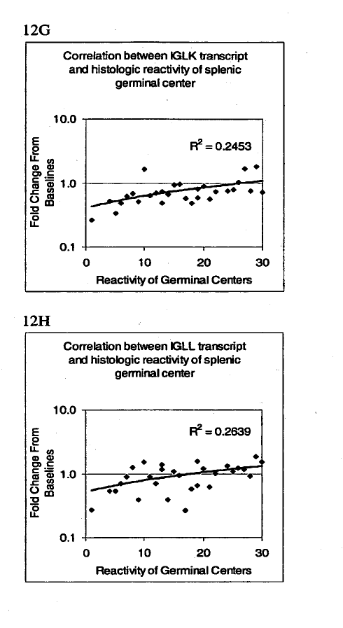

Comparisons of mean values for histologic germinal center atrophy with mean values for levels of switch transcripts in peripheral blood did not reveal significant correlations with dose (data not shown). Analyses of individual animals also were conducted by comparing the relative histologic reactivity of splenic germinal centers for an individual animal at necropsy (Week 13) to the change in levels of switch transcripts in peripheral blood from baseline (Week −2 & −1) to necropsy (Week 4 & 13). Animals that exhibited the most atrophy of germinal centers in spleen (e.g., animal 4006) also generally exhibited decreases in levels of several transcripts in peripheral blood. Quantitative analyses of these data revealed that decreases in blood levels of GLT3, CT1 and IGLL exhibited weak but positive correlations (r²˜0.2) with decreases in germinal center reactivity. Weaker correlations were observed for other transcripts. Animals that exhibited splenic germinal center atrophy generally had lower levels of GLT3 and CT1 in peripheral blood; however, animals 4503 and 4504 are exceptions in that they exhibited moderate atrophy yet high levels of GLT3 and CT1 in peripheral blood. It should be noted that these animals were also unique in that these sections contained lymphoid hyperplasia of the periarteriolar lymphoid sheath (PALS). Hyperplasia of lymphocytes is a hallmark of reactive germinal centers. It is possible that ICS did occur in the PALS of these two animals, as a compensatory response to atrophy of germinal centers and that this may be the source of disproportionately high levels of GLT3 and CT1 in peripheral blood. These morphologically exceptional spleens (4503 and 4504) heavily influence the outcomes of these comparisons. If animals 4503 and 4504 are considered outliers and are excluded from the analyses, the overall correlation between a decrease in germinal center reactivity (e.g. atrophy) and decreased levels of these transcripts in blood improved substantially for GLT3 and CT1 (r²˜0.6-0.7, FIGS. 12A & B). Excluding the same animals as outliers in reanalyses of the other transcripts also yielded higher correlations for AICDA, IGDG1 and IGLK (FIG. 12C, E, G), but not for IGHA1, IGLL and 18S (FIGS. 12D, F& H).

Comparison of Clinical Symptoms of Infection to Levels of Transcripts in Peripheral Blood

Clinical symptoms of infection (i.e. Shigellosis) were observed on Day 13 for animal 4004, Day 20 for animal 4006 and Day 29 for animal 4005. The occurrence of clinical symptoms of infection was compared to changes in levels of transcripts in peripheral blood for time-points prior to and during the onset of symptoms (Weeks −2, −1, 1, 2, 3 & 4). Slope values for transcripts in individual animals represent the magnitude of change in that transcript from Week −2 through Week 4 and were quantified by calculating the linear regression of fold changes from baseline values over this time period (Table 14). Animal 4004, for example, exhibited the largest decreases in levels of GLT3 and CT1, whereas Animal 4501 exhibited the largest decrease in AICDA, 4005 for IGHG1, 4006 for IGHA, etc. One can assess the relative predictive value of these potential safety biomarkers by ranking the slopes for each transcript and determining if the ranking value distinguishes clinically symptomatic animals (4004, 4005 & 4006) from asymptomatic animals. Ranking the slopes for GLT3 or CT1 segregated animals 4004, 4005 and 4006 into the top three positions; symptomatic animals exhibited the largest decreases in levels of GLT3 or CT1 (Table 14). Ranking slopes for AICDA, IGHG, IGHA or IGLK, segregated two of the three symptomatic animals (4005 and 4006) into the top three positions (Table 14) and the third animal (4004) segregated within the upper quintile of all animals (Table 14). Ranking slopes for IGLL, segregated two of the three symptomatic animals (4005 and 4006) within the upper quintile of all animals (Table 14). Ranking slopes for control transcripts 18S and B2MG did not segregate symptomatic from asymptomatic animals; slope values for animals 4004, 4405 and 4006 were distributed randomly among all animals examined (data not shown). These data collectively indicate that changes in levels of GLT3 or CT1 were the most accurate predictors of clinical symptoms of infection in this study.

TABLE 14
Comparison of Symptomatic Infection With Changes in Levels of Transcripts
in Peripheral Blood of Cynomolgus Monkeys From Week −2 to Week 4

| | GLT3 | | CT1 | | AICDA | | IGHG1 | |
|---|---|---|---|---|---|---|---|---|
| Rank | ID | Slope^{a} | ID | Slope | ID | Slope | ID | Slope |
| 1 | 4004 | y = −0.1708x + | 4004 | y = −0.1708x + | 4501 | y = −0.1806x + | 4005 | y = −0.1869x + |
| | | 1.4044 | | 1.4044 | | 1.2691 | | 1.2106 |
| 2 | 4005 | y = −0.1614x + | 4005 | y = −0.1673x + | 4005 | y = −0.1776x + | 4006 | y = −0.185x + |
| | | 1.3337 | | 1.3039 | | 1.1608 | | 1.2446 |
| 3 | 4006 | y = −0.1283x + | 4006 | y = −0.1283x + | 4006 | y = −0.1638x + | 4501 | y = −0.1666x + |
| | | 1.4198 | | 1.4198 | | 1.1842 | | 1.2456 |
| 4 | 2003 | y = −0.1063x + | 2503 | y = −0.1058x + | 3501 | y = −0.1479x + | 2004 | y = −0.1396x + |
| | | 1.2427 | | 1.3538 | | 1.2096 | | 1.1076 |
| 5 | 2503 | y = −0.1016x + | 2003 | y = −0.0984x + | 3503 | y = −0.142x + | 3001 | y = −0.1372x + |
| | | 1.3307 | | 1.2191 | | 1.1498 | | 1.1668 |
| 6 | 1504 | y = −0.0967x + | 2502 | y = −0.0942x + | 1001 | y = −0.1429x + | 4001 | y = −0.1235x + |
| | | 1.4225 | | 1.3131 | | 1.1696 | | 1.1496 |
| 7 | 2502 | y = −0.0961x + | 1504 | y = −0.0821x + | 1506 | y = −0.1105x + | 4004 | y = −0.1169x + |
| | | 1.3434 | | 1.3969 | | 1.0279 | | 1.2734 |
| 8 | 2501 | y = −0.0844x + | 3501 | y = −0.075x + | 2003 | y = −0.1015x + | 2504 | y = −0.1153x + |
| | | 1.2891 | | 1.4244 | | 0.9907 | | 1.0351 |
| 9 | 1506 | y = −0.0834x + | 2004 | y = −0.072x + | 3502 | y = −0.0945x + | 1006 | y = −0.1013x + |
| | | 1.313 | | 1.2363 | | 1.2817 | | 1.3697 |
| 10 | 3002 | y = −0.0692x + | 2501 | y = −0.0725x + | 2503 | y = −0.0928x + | 3004 | y = −0.0999x + |
| | | 1.2162 | | 1.2351 | | 1.1107 | | 1.0101 |
| 11 | 3501 | y = −0.0685x + | 1506 | y = −0.0668x + | 2004 | y = −0.0888x + | 1002 | y = −0.0928x + |
| | | 1.4031 | | 1.247 | | 1.1156 | | 0.9635 |
| 12 | 2004 | y = −0.0668x + | 3002 | y = −0.0659x + | 1504 | y = −0.0841x + | 3503 | y = −0.0873x + |
| | | 1.213 | | 1.1969 | | 0.9872 | | 1.0192 |
| 13 | 2504 | y = −0.0607x + | 2504 | y = −0.0518x + | 4004 | y = −0.0748x + | 3003 | y = −0.0866x + |
| | | 1.163 | | 1.1373 | | 1.2671 | | 1.05 |
| 14 | 4506 | y = −0.0477x + | 4506 | y = −0.0477x + | 1005 | y = −0.0703x + | 1506 | y = −0.0842x + |
| | | 1.2385 | | 1.2385 | | 1.0747 | | 1.0152 |
| 15 | 1005 | y = −0.0467x + | 4504 | y = −0.0462x + | 4504 | y = −0.0681x + | 1504 | y = −0.0808x + |
| | | 1.187 | | 1.0621 | | 0.9775 | | 1.0518 |
| 16 | 4504 | y = −0.0462x + | 4001 | y = −0.0413x + | 4502 | y = −0.065x + | 3501 | y = −0.0766x + |
| | | 1.0621 | | 1.1172 | | 1.1119 | | 1.1437 |
| 17 | 4001 | y = −0.0413x + | 3503 | y = −0.0395x + | 1006 | y = −0.058x + | 4002 | y = −0.0559x + |
| | | 1.1172 | | 1.1433 | | 1.3888 | | 0.9637 |
| 18 | 3503 | y = −0.033x + | 3001 | y = −0.028x + | 4002 | y = −0.0579x + | 4504 | y = −0.0559x + |
| | | 1.1262 | | 1.1417 | | 0.9865 | | 0.9637 |
| 19 | 3001 | y = −0.0337x + | 4502 | y = −0.0282x + | 4001 | y = −0.0564x + | 2003 | y = −0.0509x + |
| | | 1.1719 | | 1.098 | | 1.1328 | | 0.9053 |
| 20 | 4502 | y = −0.0282x + | 1005 | y = −0.0226x + | 1002 | y = −0.0546x + | 1001 | y = −0.0507x + |
| | | 1.098 | | 1.092 | | 0.9215 | | 1.0576 |
| 21 | 1004 | y = −0.0238x + | 1004 | y = −0.0178x + | 3504 | y = −0.0535x + | 4502 | y = −0.0413x + |
| | | 1.0586 | | 1.0342 | | 0.9562 | | 1.003 |
| 22 | 2002 | y = −0.0178x + | 1002 | y = −0.0151x + | 3001 | y = −0.0451x + | 2503 | y = −0.037x + |
| | | 1.0078 | | 1.0865 | | 1.0988 | | 0.9933 |
| 23 | 1002 | y = −0.0148x + | 2002 | y = −0.0109x + | 3003 | y = −0.0394x + | 1005 | y = −0.0375x + |
| | | 1.1 | | 0.9656 | | 1.0028 | | 0.9391 |
| 24 | 1501 | y = −0.0055x + | 1502 | y = −0.004x + | 4505 | y = −0.0359x + | 1004 | y = −0.0325x + |
| | | 1.067 | | 1.1115 | | 0.9678 | | 0.9241 |
| 25 | 1505 | y = 0.0026x + | 1501 | y = −0.0042x + | 1004 | y = −0.031x + | 1505 | y = −0.0122x + |
| | | 0.9714 | | 1.0566 | | 0.9311 | | 0.806 |
| 26 | 1502 | y = 0.0034x + | 1001 | y = 0.0101x + | 1503 | y = −0.021x + | 3504 | y = −0.006x + |
| | | 1.0817 | | 1.0036 | | 0.8448 | | 0.8587 |
| 27 | 1003 | y = 0.0039x + | 1505 | y = 0.0149x + | 1501 | y = −0.0077x + | 1501 | y = 0.0019x + |
| | | 0.9103 | | 0.9614 | | 0.9347 | | 0.8438 |
| 28 | 4501 | y = 0.0189x + | 1003 | y = 0.0165x + | 2002 | y = 0.0011x + | 4505 | y = 0.0021x + |
| | | 0.993 | | 0.8595 | | 0.0893 | | 0.9353 |
| 29 | 3504 | y = 0.0233x + | 4501 | y = 0.0189x + | 4503 | y = 0.0019x + | 4003 | y = 0.0021x + |
| | | 0.8593 | | 0.993 | | 0.8963 | | 1.0885 |
| 30 | 1001 | y = 0.0255x + | 3504 | y = 0.0227x + | 1502 | y = 0.0083x + | 2501 | y = 0.0072x + |
| | | 0.9566 | | 0.8557 | | 0.9184 | | 1.1496 |
| 31 | 4002 | y = 0.0264x + | 3004 | y = 0.0236x + | 2101 | y = 0.0084x + | 1502 | y = 0.0233x + |
| | | 0.9284 | | 0.9067 | | 0.9523 | | 0.889 |
| 32 | 4505 | y = 0.0266x + | 4002 | y = 0.0264x + | 2504 | y = 0.012x + | 3002 | y = 0.0308x + |
| | | 0.9059 | | 0.9284 | | 0.8623 | | 1.1 |
| 33 | 3004 | y = 0.029x + | 4505 | y = 0.0266x + | 1003 | y = 0.0254x + | 3502 | y = −0.0342x + |
| | | 0.8888 | | 0.9059 | | 1.0253 | | 1.2773 |
| 34 | 2101 | y = 0.0325x + | 4003 | y = 0.0382x + | 2501 | y = 0.0284x + | 1003 | y = 0.0375x + |
| | | 0.9206 | | 0.8779 | | 1.0457 | | 0.883 |
| 35 | 4003 | y = 0.0382x + | 1006 | y = 0.0391x + | 1505 | y = 0.0345x + | 2002 | y = 0.04x + |
| | | 0.8779 | | 0.8201 | | 0.8829 | | 0.859 |
| 36 | 1006 | y = 0.0494x + | 2101 | y = 0.0485x + | 3002 | y = 0.03x + | 2502 | y = 0.0593x + |
| | | 0.7819 | | 0.8757 | | 1.1414 | | 0.8762 |
| 37 | 3502 | y = 0.0561x + | 1503 | y = 0.0627x + | 3004 | y = 0.043x + | 4506 | y = 0.066x + |
| | | 0.7711 | | 0.8778 | | 0.7108 | | 1.0079 |
| 38 | 1503 | y = 0.0585x + | 3502 | y = 0.0707x + | 4003 | y = 0.0681x + | 2101 | y = 0.0831x + |
| | | 0.8814 | | 0.7175 | | 1.0699 | | 0.8645 |
| 39 | 3003 | y = 0.1291x + | 4505 | y = 0.0266x + | 4506 | y = 0.2193x + | 4503 | y = 0.1364x + |
| | | 0.5302 | | 0.9059 | | 0.9298 | | 0.6808 |
| 40 | 4503 | y = 0.1299x + | 4506 | y = −0.0477x + | 2502 | y = 0.3303x + | 1503 | y = 0.361x + |
| | | 0.5066 | | 1.2385 | | 0.1932 | | 0.408 |

| | IGHA | | IGLK | | IGLL | |
|---|---|---|---|---|---|---|
| Rank | ID | Slope | ID | Slope | ID | Slope |
| 1 | 4006 | y = −0.218x + | 4005 | y = −0.1869x + | 3504 | y = −0.1992x + |
| | | 1.2841 | | 1.2106 | | 1.1967 |
| 2 | 4005 | y = −0.1997x + | 4006 | y = −0.185x + | 3503 | y = −0.195x + |
| | | 1.1456 | | 1.2446 | | 1.1923 |
| 3 | 1504 | y = −0.15x + | 4501 | y = −0.1666x + | 4001 | y = −0.1887x + |
| | | 1.5727 | | 1.2456 | | 1.2514 |
| 4 | 4506 | y = −0.127x + | 2004 | y = −0.1363x + | 2004 | y = −0.1851x + |
| | | 1.1347 | | 1.1752 | | 1.2243 |
| 5 | 3501 | y = −0.1278x + | 3001 | y = −0.1275x + | 4006 | y = −0.1755x + |
| | | 1.1921 | | 1.0991 | | 1.2353 |
| 6 | 3001 | y = −0.1265x + | 1006 | y = −0.1269x + | 4005 | y = −0.1625x + |
| | | 1.0822 | | 1.3542 | | 1.1408 |
| 7 | 3004 | y = −0.1245x + | 4001 | y = −0.1235x + | 1504 | y = −0.1218x + |
| | | 1.0928 | | 1.1496 | | 1.1602 |
| 8 | 1002 | y = −0.1116x + | 4004 | y = −0.1169x + | 3004 | y = −0.1013x + |
| | | 0.9902 | | 1.2734 | | 1.0844 |
| 9 | 2003 | y = −0.1015x + | 3003 | y = −0.111x + | 3001 | y = −0.0967x + |
| | | 0.9907 | | 1.1087 | | 1.0688 |
| 10 | 2504 | y = −0.0907x + | 3503 | y = −0.1096x + | 3502 | y = −0.094x + |
| | | 0.9453 | | 1.1083 | | 1.7009 |
| 11 | 1006 | y = −0.08x + | 1504 | y = −0.0964x + | 3003 | y = −0.0939x + |
| | | 1.1774 | | 1.0503 | | 1.1456 |
| 12 | 2004 | y = −0.0888x + | 3501 | y = −0.0911x + | 3501 | y = −0.0834x + |
| | | 1.1156 | | 1.148 | | 1.1457 |
| 13 | 3003 | y = −0.0864x + | 3004 | y = −0.0911x + | 1501 | y = −0.0828x + |
| | | 1.0732 | | 1.1056 | | 0.8933 |
| 14 | 4501 | y = −0.0858x + | 3504 | y = −0.0871x + | 1003 | y = 0.0817x + |
| | | 1.1437 | | 1.0592 | | 1.094 |
| 15 | 4001 | y = −0.0851x + | 3502 | y = −0.0848x + | 4501 | y = −0.075x + |
| | | 1.0022 | | 1.2028 | | 1.0908 |
| 16 | 4002 | y = −0.0677x + | 1506 | y = −0.0837x + | 4504 | y = −0.0655x + |
| | | 0.9776 | | 1.0692 | | 1.0494 |
| 17 | 1501 | y = −0.0667x + | 1002 | y = −0.07x + | 1505 | y = −0.0543x + |
| | | 0.9328 | | 0.9669 | | 0.883 |
| 18 | 2501 | y = −0.0609x + | 2003 | y = −0.0737x + | 2503 | y = −0.0458x + |
| | | 1.6551 | | 1.0432 | | 0.9905 |
| 19 | 4502 | y = −0.0505x + | 3002 | y = −0.0734x + | 4503 | y = −0.0453x + |
| | | 0.8973 | | 1.2265 | | 1.0088 |
| 20 | 1502 | y = −0.0434x + | 1005 | y = −0.072x + | 2003 | y = −0.0408x + |
| | | 1.0224 | | 1.0893 | | 0.9189 |
| 21 | 4505 | y = −0.0408x + | 4002 | y = −0.0559x + | 1002 | y = −0.0398x + |
| | | 0.9989 | | 0.9637 | | 0.8941 |
| 22 | 1506 | y = −0.017x + | 1501 | y = −0.0459x + | 1506 | y = −0.0356x + |
| | | 1.1032 | | 0.9372 | | 0.971 |
| 23 | 4503 | y = −0.0176x + | 4504 | y = −0.044x + | 2501 | y = −0.0332x + |
| | | 0.73 | | 1.0155 | | 1.1874 |
| 24 | 3002 | y = −0.0164x + | 4502 | y = −0.0413x + | 4502 | y = −0.0332x + |
| | | 1.272 | | 1.003 | | 0.9682 |
| 25 | 4004 | y = −0.0164x + | 2101 | y = −0.0383x + | 1503 | y = −0.0066x + |
| | | 0.8648 | | 1.0457 | | 0.8924 |
| 26 | 1005 | y = −0.0156x + | 2504 | y = −0.0318x + | 1005 | y = −0.0025x + |
| | | 1.0548 | | 0.8724 | | 1.0638 |
| 27 | 1505 | y = −0.0006x + | 1505 | y = −0.027x + | 1001 | y = 0.0045x + |
| | | 0.7524 | | 0.8553 | | 0.9491 |
| 28 | 4504 | y = 0.0002x + | 1004 | y = −0.0258x + | 1006 | y = 0.0321x + |
| | | 0.8913 | | 0.9499 | | 1.3645 |
| 29 | 2002 | y = 0.0011x + | 1003 | y = −0.0144x + | 2002 | y = 0.0391x + |
| | | 0.0893 | | 0.9628 | | 0.8381 |
| 30 | 2101 | y = 0.028x + | 1502 | y = −0.0136x + | 4506 | y = 0.0453x + |
| | | 0.967 | | 0.9466 | | 1.0041 |
| 31 | 1001 | y = 0.0299x + | 1001 | y = 0.0011x + | 4002 | y = 0.0574x + |
| | | 1.1096 | | 1.0977 | | 0.8364 |
| 32 | 3503 | y = 0.0417x + | 4505 | y = 0.0021x + | 4505 | y = 0.0859x + |
| | | 0.7948 | | 0.9353 | | 0.8101 |
| 33 | 2503 | y = 0.064x + | 4003 | y = 0.0021x + | 4004 | y = 0.0905x + |
| | | 0.7354 | | 1.0885 | | 0.6369 |
| 34 | 3502 | y = 0.0654x + | 2002 | y = 0.0111x + | 1502 | y = 0.0979x + |
| | | 0.9361 | | 0.8426 | | 0.8676 |
| 35 | 2502 | y = 0.0881x + | 2501 | y = 0.0201x + | 3002 | y = 0.1094x + |
| | | 0.7674 | | 1.3507 | | 0.9183 |
| 36 | 1003 | y = 0.1602x + | 1503 | y = 0.0328x + | 2101 | y = 0.1554x + |
| | | 0.7026 | | 0.8389 | | 0.8416 |
| 37 | 3504 | y = 0.1823x + | 4506 | y = 0.066x + | 2502 | y = 0.1794x + |
| | | 0.6623 | | 1.0079 | | 0.4851 |
| 38 | 1004 | y = 0.203x + | 2502 | y = 0.1217x + | 2504 | y = 0.2394x + |
| | | 0.6491 | | 0.7379 | | 0.2681 |
| 39 | 4003 | y = 0.398x + | 4503 | y = 0.1364x + | 1004 | y = 0.3365x + |
| | | 0.4401 | | 0.6808 | | 0.3899 |
| 40 | 1503 | y = 0.402x + | | | 4003 | y = 0.3682x + |
| | | 0.2221 | | | | 0.4393 |

ID = animal identification number.
Note: Bold font indicates animals that exhibited clinical symptoms of infection (observed on Day 13 for Animal 4004, Day 20 for Animal 4006, and Day 29 for Animal 4005).
^{a}Slope values for transcripts in individual animals represent the magnitude of potential changes in that transcript from Week −2 through Week 4 and were quantified by calculating the linear regression of fold change from baseline values over this time period. Slope values are listed in ascending order for each transcript and the corresponding animal identification number is included.

Animals with largest decreases in blood switch transcripts over time were those that developed clinical symptoms of infection. Of particular interest, decreases in levels of GLT3 and CT1 in peripheral blood preceded the onset of clinical symptoms of infection in animal 4005 by at least two weeks (FIG. 8B). Similar pre-infection trends in the level of GLT3 and CT1 in peripheral blood appear to have occurred in animals 4004 and 4006. These data, combined with the knowledge that immunoglobulin ICS is required for resistance to infection, collectively indicate that decreases in GLT3 and CT1 are antecedent markers of infection. We therefore conclude that GLT3 and CT1 are useful predictive markers of clinical infection.

### Conclusions

High exposures to Test Agent A caused atrophy of germinal centers, inhibition of antibody responses and bacterial infection. Test Agent A inhibits differentiation of B cells and antibody responses de novo. Change in levels of switch transcripts in Cyno tissue or peripheral blood correlate with germinal center atrophy, TDAR and with clinical symptoms of infection. Changes in levels of switch transcripts may be a noninvasive and antecedent marker of immunosuppression in clinical subjects.

All documents cited throughout this application including references, pending patent applications and published patents, are hereby expressly incorporated herein by reference in their entirety.

### EQUIVALENTS

Although preferred embodiments of the invention have been described using specific terms, such description are for illustrative purposes only, and it is to be understood that changes and variations may be made without departing from the spirit or scope of the invention. Those skilled in the art will recognize, or be able to ascertain using no more than routine experimentation, many equivalents of the specific embodiments of the invention described herein. Such equivalents are intended to be encompassed by the following claims.

1.-4. (canceled)

5. A method for determining whether a patient is susceptible to a disorder selected from the group consisting of opportunistic infection and lymphoma, comprising:
a. Obtaining a sample from the patient prior to treating with a therapeutic regimen;
b. Contacting the sample with a probe to detect a marker selected from the group consisting of a germline switch transcript, a circle transcript and a DNA recombination effector;
c. Measuring the amount of probe bound to the sample to determine the amount of the marker in the sample;
d. Treating the patient with the therapeutic regimen;
e. Obtaining an additional sample from the patient after some treatment;
f and g. Repeating steps b and c on the additional sample; and
h. Determining that the patient is susceptible to the disorder if the amount of marker in the sample after some treatment is less than the level in the sample before treatment.

6. A method for determining whether a patient is susceptible to a disorder selected from the group consisting of opportunistic infection and lymphoma, comprising:
a. Obtaining a sample from the patient;
b. Contacting the sample with a probe to detect a marker selected from the group consisting of a germline switch transcript, a circle transcript and a DNA recombination effector;
c. Measuring the amount of probe bound to the sample to determine the amount of marker in the sample; and
d. Determining that the patient is susceptible to the disorder if the amount of marker is less than a pre-determined standard.

7. A method for determining recovery of a patient from immunosuppression or determining whether a vaccination is efficacious comprising:
a. Obtaining a sample from the patient;
b. Contacting the sample with a probe to detect a marker selected from the group consisting of a germline switch transcript, a circle transcript and a DNA recombination effector;
c. Measuring the amount of probe bound to the sample to determine the amount of marker in the sample; and
d. Determining that the patient has recovered from immunosuppression or has had an efficacious vaccination if the amount of the marker is more than a pre-determined standard.

8.-12. (canceled)

13. The method of claim 5, wherein the sample is a peripheral blood sample.

14. The method of claim 5, further comprising enriching the sample for B cells.

15. The method of claim 5, wherein the level of at least two markers is determined.

16. The method of claim 5, wherein the amount of RNA of the biomarker in the sample is determined.

17. The method of claim 16, wherein the RNA in the sample is stabilized.

18. The method of claim 5, wherein the marker is selected from the group consisting of:
germline transcript mu (GLT-μ), circle transcript containing gamma 1 (CT-γ1), circle transcript containing gamma 2 (CT-γ2), activation-induced cytidine deaminase (AID), immunoglobulin heavy chain type G1 (IGHG1), and immunoglobulin heavy chain type A2 (IGHA2).

19. A method of deciding whether to pay for continued treatment of a chronic immune disorder comprising:
a) obtaining the expression level of a biomarker or a biomarker set comprising a marker selected from the group consisting of a germline switch transcript, a circle transcript and a DNA recombination effector; and
b) authorizing payment if the expression level indicates that the patient is not immunosuppressed.

20. The method of claim 19, wherein the marker is selected from the group consisting of germline transcript mu (GLT-μ), circle transcript containing gamma 1 (CT-γ1), circle transcript containing gamma 2 (CT-γ2), activation-induced cytidine deaminase (AID), immunoglobulin heavy chain type G1 (IGHG1), and immunoglobulin heavy chain type A2 (IGHA2).

21. A method for screening to identify an immunomodulator comprising:
a) contacting a sample comprising B cells with a test agent;
b) measuring the level of expression of a marker selected from the group consisting of a germline switch transcript, a circle transcript and a DNA recombination effector; and
c) determining that the test agent is an immunomodulator if the level of expression of the marker is significantly different than the level in a sample that was not contacted with the test agent.
